SEQUENCE LISTING (340 sequences)

SEQ ID NO: 1 - RNA, 21 nt, synthetic construct
  source 1..21: mol_type = other RNA
ccagcatgccggcttccgcgg

SEQ ID NO: 2 - RNA, 21 nt, synthetic construct
  source 1..21: mol_type = other RNA
tccagcatgccggcttccgcg

SEQ ID NO: 3 - RNA, 21 nt, synthetic construct
  source 1..21: mol_type = other RNA
ctccagcatgccggcttccgc

SEQ ID NO: 4 - RNA, 21 nt, synthetic construct
  source 1..21: mol_type = other RNA
ttctccagcatgccggcttcc

SEQ ID NO: 5 - RNA, 21 nt, synthetic construct
  source 1..21: mol_type = other RNA
cttctccagcatgccggcttc

SEQ ID NO: 6 - RNA, 21 nt, synthetic construct
  source 1..21: mol_type = other RNA
gcttctccagcatgccggctt

SEQ ID NO: 7 - RNA, 21 nt, synthetic construct
  source 1..21: mol_type = other RNA
agcttctccagcatgccggct

SEQ ID NO: 8 - RNA, 21 nt, synthetic construct
  source 1..21: mol_type = other RNA
cagcttctccagcatgccggc

SEQ ID NO: 9 - RNA, 21 nt, synthetic construct
  source 1..21: mol_type = other RNA
gtgtgacctcatgaatcgcat

SEQ ID NO: 10 - RNA, 21 nt, synthetic construct
  source 1..21: mol_type = other RNA
tgtgtgacctcatgaatcgca

SEQ ID NO: 11 - RNA, 21 nt, synthetic construct
  source 1..21: mol_type = other RNA
ttgtgtgacctcatgaatcgc

SEQ ID NO: 12 - RNA, 21 nt, synthetic construct
  source 1..21: mol_type = other RNA
cttgtgtgacctcatgaatcg

SEQ ID NO: 13 - RNA, 21 nt, synthetic construct
  source 1..21: mol_type = other RNA
acttgtgtgacctcatgaatc

SEQ ID NO: 14 - RNA, 21 nt, synthetic construct
  source 1..21: mol_type = other RNA
cacttgtgtgacctcatgaat

SEQ ID NO: 15 - RNA, 21 nt, synthetic construct
  source 1..21: mol_type = other RNA
acacttgtgtgacctcatgaa

SEQ ID NO: 16 - RNA, 21 nt, synthetic construct
  source 1..21: mol_type = other RNA
taacacttgtgtgacctcatg

SEQ ID NO: 17 - RNA, 21 nt, synthetic construct
  source 1..21: mol_type = other RNA
cataacacttgtgtgacctca

SEQ ID NO: 18 - RNA, 21 nt, synthetic construct
  source 1..21: mol_type = other RNA
tcataacacttgtgtgacctc

SEQ ID NO: 19 - RNA, 21 nt, synthetic construct
  source 1..21: mol_type = other RNA
atgtcataacacttgtgtgac

SEQ ID NO: 20 - RNA, 21 nt, synthetic construct
  source 1..21: mol_type = other RNA
acgatgtcataacacttgtgt

SEQ ID NO: 21 - RNA, 21 nt, synthetic construct
  source 1..21: mol_type = other RNA
aacgatgtcataacacttgtg

SEQ ID NO: 22 - RNA, 21 nt, synthetic construct
  source 1..21: mol_type = other RNA
gaacgatgtcataacacttgt

SEQ ID NO: 23 - RNA, 21 nt, synthetic construct
  source 1..21: mol_type = other RNA
tggaacgatgtcataacactt

SEQ ID NO: 24 - RNA, 21 nt, synthetic construct
  source 1..21: mol_type = other RNA
ttggaacgatgtcataacact

SEQ ID NO: 25 - RNA, 21 nt, synthetic construct
  source 1..21: mol_type = other RNA
gttggaacgatgtcataacac

SEQ ID NO: 26 - RNA, 21 nt, synthetic construct
  source 1..21: mol_type = other RNA
ggttggaacgatgtcataaca

SEQ ID NO: 27 - RNA, 21 nt, synthetic construct
  source 1..21: mol_type = other RNA
tggttggaacgatgtcataac

SEQ ID NO: 28 - RNA, 21 nt, synthetic construct
  source 1..21: mol_type = other RNA
ctggttggaacgatgtcataa

SEQ ID NO: 29 - RNA, 21 nt, synthetic construct
  source 1..21: mol_type = other RNA
actggttggaacgatgtcata

SEQ ID NO: 30 - RNA, 21 nt, synthetic construct
  source 1..21: mol_type = other RNA
aactggttggaacgatgtcat

SEQ ID NO: 31 - RNA, 21 nt, synthetic construct
  source 1..21: mol_type = other RNA
gaactggttggaacgatgtca

SEQ ID NO: 32 - RNA, 21 nt, synthetic construct
  source 1..21: mol_type = other RNA
tgaactggttggaacgatgtc

SEQ ID NO: 33 - RNA, 21 nt, synthetic construct
  source 1..21: mol_type = other RNA
ttgaactggttggaacgatgt

SEQ ID NO: 34 - RNA, 21 nt, synthetic construct
  source 1..21: mol_type = other RNA
tttgaactggttggaacgatg

SEQ ID NO: 35 - RNA, 21 nt, synthetic construct
  source 1..21: mol_type = other RNA
ctttgaactggttggaacgat

SEQ ID NO: 36 - RNA, 21 nt, synthetic construct
  source 1..21: mol_type = other RNA
gctttgaactggttggaacga

SEQ ID NO: 37 - RNA, 21 nt, synthetic construct
  source 1..21: mol_type = other RNA
agctttgaactggttggaacg

SEQ ID NO: 38 - RNA, 21 nt, synthetic construct
  source 1..21: mol_type = other RNA
caagctttgaactggttggaa

SEQ ID NO: 39 - RNA, 21 nt, synthetic construct
  source 1..21: mol_type = other RNA
acaagctttgaactggttgga

SEQ ID NO: 40 - RNA, 21 nt, synthetic construct
  source 1..21: mol_type = other RNA
cccactgatagggtcaataac

SEQ ID NO: 41 - RNA, 21 nt, synthetic construct
  source 1..21: mol_type = other RNA
tcccactgatagggtcaataa

SEQ ID NO: 42 - RNA, 21 nt, synthetic construct
  source 1..21: mol_type = other RNA
ttcccactgatagggtcaata

SEQ ID NO: 43 - RNA, 21 nt, synthetic construct
  source 1..21: mol_type = other RNA
aacttgaggattcttttgtgg

SEQ ID NO: 44 - RNA, 21 nt, synthetic construct
  source 1..21: mol_type = other RNA
ggaggaacttgaggattcttt

SEQ ID NO: 45 - RNA, 21 nt, synthetic construct
  source 1..21: mol_type = other RNA
taaaaagctggaggaacttga

SEQ ID NO: 46 - RNA, 21 nt, synthetic construct
  source 1..21: mol_type = other RNA
acataaaaagctggaggaact

SEQ ID NO: 47 - RNA, 21 nt, synthetic construct
  source 1..21: mol_type = other RNA
gacataaaaagctggaggaac

SEQ ID NO: 48 - RNA, 21 nt, synthetic construct
  source 1..21: mol_type = other RNA
agacataaaaagctggaggaa

SEQ ID NO: 49 - RNA, 21 nt, synthetic construct
  source 1..21: mol_type = other RNA
cttcatgaaggcaggctttgg

SEQ ID NO: 50 - RNA, 21 nt, synthetic construct
  source 1..21: mol_type = other RNA
caggttctgcttcatgaaggc

SEQ ID NO: 51 - RNA, 21 nt, synthetic construct
  source 1..21: mol_type = other RNA
tccaggttctgcttcatgaag

SEQ ID NO: 52 - RNA, 21 nt, synthetic construct
  source 1..21: mol_type = other RNA
atccaggttctgcttcatgaa

SEQ ID NO: 53 - RNA, 21 nt, synthetic construct
  source 1..21: mol_type = other RNA
agctcatccaggttctgcttc

SEQ ID NO: 54 - RNA, 21 nt, synthetic construct
  source 1..21: mol_type = other RNA
aagctcatccaggttctgctt

SEQ ID NO: 55 - RNA, 21 nt, synthetic construct
  source 1..21: mol_type = other RNA
caagctcatccaggttctgct

SEQ ID NO: 56 - RNA, 21 nt, synthetic construct
  source 1..21: mol_type = other RNA
ccaagctcatccaggttctgc

SEQ ID NO: 57 - RNA, 21 nt, synthetic construct
  source 1..21: mol_type = other RNA
acaacttttcctgactcatcc

SEQ ID NO: 58 - RNA, 21 nt, synthetic construct
  source 1..21: mol_type = other RNA
tacaacttttcctgactcatc

SEQ ID NO: 59 - RNA, 21 nt, synthetic construct
  source 1..21: mol_type = other RNA
ctacaacttttcctgactcat

SEQ ID NO: 60 - RNA, 21 nt, synthetic construct
  source 1..21: mol_type = other RNA
tatctacaacttttcctgact

SEQ ID NO: 61 - RNA, 21 nt, synthetic construct
  source 1..21: mol_type = other RNA
ggaataaatatctacaacttt

SEQ ID NO: 62 - RNA, 21 nt, synthetic construct
  source 1..21: mol_type = other RNA
tggaataaatatctacaactt

SEQ ID NO: 63 - RNA, 21 nt, synthetic construct
  source 1..21: mol_type = other RNA
ttggaataaatatctacaact

SEQ ID NO: 64 - RNA, 21 nt, synthetic construct
  source 1..21: mol_type = other RNA
agcaagattaattacatcaaa

SEQ ID NO: 65 - RNA, 21 nt, synthetic construct
  source 1..21: mol_type = other RNA
ctcagcagcaagattaattac

SEQ ID NO: 66 - RNA, 21 nt, synthetic construct
  source 1..21: mol_type = other RNA
tctcagcagcaagattaatta

SEQ ID NO: 67 - RNA, 21 nt, synthetic construct
  source 1..21: mol_type = other RNA
ttctcagcagcaagattaatt

SEQ ID NO: 68 - RNA, 21 nt, synthetic construct
  source 1..21: mol_type = other RNA
ttttctcagcagcaagattaa

SEQ ID NO: 69 - RNA, 21 nt, synthetic construct
  source 1..21: mol_type = other RNA
ggtgtgaggatcagggcttgc

SEQ ID NO: 70 - RNA, 21 nt, synthetic construct
  source 1..21: mol_type = other RNA
tggtgtgaggatcagggcttg

SEQ ID NO: 71 - RNA, 21 nt, synthetic construct
  source 1..21: mol_type = other RNA
acctgctggtgtgaggatcag

SEQ ID NO: 72 - RNA, 21 nt, synthetic construct
  source 1..21: mol_type = other RNA
cgtctacattcacggcggtca

SEQ ID NO: 73 - RNA, 21 nt, synthetic construct
  source 1..21: mol_type = other RNA
gcgtctacattcacggcggtc

SEQ ID NO: 74 - RNA, 21 nt, synthetic construct
  source 1..21: mol_type = other RNA
ggcgtctacattcacggcggt

SEQ ID NO: 75 - RNA, 21 nt, synthetic construct
  source 1..21: mol_type = other RNA
gggcgtctacattcacggcgg

SEQ ID NO: 76 - RNA, 21 nt, synthetic construct
  source 1..21: mol_type = other RNA
ttgcagccagtgttcatgagg

SEQ ID NO: 77 - RNA, 21 nt, synthetic construct
  source 1..21: mol_type = other RNA
ctgaatcttcaagcacataaa

SEQ ID NO: 78 - RNA, 21 nt, synthetic construct
  source 1..21: mol_type = other RNA
cctgaatcttcaagcacataa

SEQ ID NO: 79 - RNA, 21 nt, synthetic construct
  source 1..21: mol_type = other RNA
gcctgaatcttcaagcacata

SEQ ID NO: 80 - RNA, 21 nt, synthetic construct
  source 1..21: mol_type = other RNA
agcctgaatcttcaagcacat

SEQ ID NO: 81 - RNA, 21 nt, synthetic construct
  source 1..21: mol_type = other RNA
actttaatgacatacagcatt

SEQ ID NO: 82 - RNA, 21 nt, synthetic construct
  source 1..21: mol_type = other RNA
gcactttaatgacatacagca

SEQ ID NO: 83 - RNA, 21 nt, synthetic construct
  source 1..21: mol_type = other RNA
cagtgcactttaatgacatac

SEQ ID NO: 84 - RNA, 21 nt, synthetic construct
  source 1..21: mol_type = other RNA
acagtgcactttaatgacata

SEQ ID NO: 85 - RNA, 21 nt, synthetic construct
  source 1..21: mol_type = other RNA
cacagtgcactttaatgacat

SEQ ID NO: 86 - RNA, 21 nt, synthetic construct
  source 1..21: mol_type = other RNA
acacagtgcactttaatgaca

SEQ ID NO: 87 - RNA, 21 nt, synthetic construct
  source 1..21: mol_type = other RNA
gacacagtgcactttaatgac

SEQ ID NO: 88 - RNA, 21 nt, synthetic construct
  source 1..21: mol_type = other RNA
ggacacagtgcactttaatga

SEQ ID NO: 89 - RNA, 21 nt, synthetic construct
  source 1..21: mol_type = other RNA
aggacacagtgcactttaatg

SEQ ID NO: 90 - RNA, 21 nt, synthetic construct
  source 1..21: mol_type = other RNA
caggacacagtgcactttaat

SEQ ID NO: 91 - RNA, 21 nt, synthetic construct
  source 1..21: mol_type = other RNA
tcaggacacagtgcactttaa

SEQ ID NO: 92 - RNA, 21 nt, synthetic construct
  source 1..21: mol_type = other RNA
agtgtcaacattttcagagca

SEQ ID NO: 93 - RNA, 19 nt, synthetic construct
  source 1..19: mol_type = other RNA
agcatgccggcttccgcgg

SEQ ID NO: 94 - RNA, 19 nt, synthetic construct
  source 1..19: mol_type = other RNA
cagcatgccggcttccgcg

SEQ ID NO: 95 - RNA, 19 nt, synthetic construct
  source 1..19: mol_type = other RNA
ccagcatgccggcttccgc

SEQ ID NO: 96 - RNA, 19 nt, synthetic construct
  source 1..19: mol_type = other RNA
ggttggaacgatgtcataa

SEQ ID NO: 97 - RNA, 19 nt, synthetic construct
  source 1..19: mol_type = other RNA
ctggttggaacgatgtcat

SEQ ID NO: 98 - RNA, 19 nt, synthetic construct
  source 1..19: mol_type = other RNA
actggttggaacgatgtca

SEQ ID NO: 99 - RNA, 19 nt, synthetic construct
  source 1..19: mol_type = other RNA
aactggttggaacgatgtc

SEQ ID NO: 100 - RNA, 19 nt, synthetic construct
  source 1..19: mol_type = other RNA
gaactggttggaacgatgt

SEQ ID NO: 101 - RNA, 19 nt, synthetic construct
  source 1..19: mol_type = other RNA
cgtctacattcacggcggt

SEQ ID NO: 102 - RNA, 19 nt, synthetic construct
  source 1..19: mol_type = other RNA
gcgtctacattcacggcgg

SEQ ID NO: 103 - RNA, 21 nt, synthetic construct
  source 1..21: mol_type = other RNA
ccgcggaagccggcatgctgg

SEQ ID NO: 104 - RNA, 21 nt, synthetic construct
  source 1..21: mol_type = other RNA
cgcggaagccggcatgctgga

SEQ ID NO: 105 - RNA, 21 nt, synthetic construct
  source 1..21: mol_type = other RNA
gcggaagccggcatgctggag

SEQ ID NO: 106 - RNA, 21 nt, synthetic construct
  source 1..21: mol_type = other RNA
ggaagccggcatgctggagaa

SEQ ID NO: 107 - RNA, 21 nt, synthetic construct
  source 1..21: mol_type = other RNA
gaagccggcatgctggagaag

SEQ ID NO: 108 - RNA, 21 nt, synthetic construct
  source 1..21: mol_type = other RNA
aagccggcatgctggagaagc

SEQ ID NO: 109 - RNA, 21 nt, synthetic construct
  source 1..21: mol_type = other RNA
agccggcatgctggagaagct

SEQ ID NO: 110 - RNA, 21 nt, synthetic construct
  source 1..21: mol_type = other RNA
gccggcatgctggagaagctg

SEQ ID NO: 111 - RNA, 21 nt, synthetic construct
  source 1..21: mol_type = other RNA
atgcgattcatgaggtcacac

SEQ ID NO: 112 - RNA, 21 nt, synthetic construct
  source 1..21: mol_type = other RNA
tgcgattcatgaggtcacaca

SEQ ID NO: 113 - RNA, 21 nt, synthetic construct
  source 1..21: mol_type = other RNA
gcgattcatgaggtcacacaa

SEQ ID NO: 114 - RNA, 21 nt, synthetic construct
  source 1..21: mol_type = other RNA
cgattcatgaggtcacacaag

SEQ ID NO: 115 - RNA, 21 nt, synthetic construct
  source 1..21: mol_type = other RNA
gattcatgaggtcacacaagt

SEQ ID NO: 116 - RNA, 21 nt, synthetic construct
  source 1..21: mol_type = other RNA
attcatgaggtcacacaagtg

SEQ ID NO: 117 - RNA, 21 nt, synthetic construct
  source 1..21: mol_type = other RNA
ttcatgaggtcacacaagtgt

SEQ ID NO: 118 - RNA, 21 nt, synthetic construct
  source 1..21: mol_type = other RNA
catgaggtcacacaagtgtta

SEQ ID NO: 119 - RNA, 21 nt, synthetic construct
  source 1..21: mol_type = other RNA
tgaggtcacacaagtgttatg

SEQ ID NO: 120 - RNA, 21 nt, synthetic construct
  source 1..21: mol_type = other RNA
gaggtcacacaagtgttatga

SEQ ID NO: 121 - RNA, 21 nt, synthetic construct
  source 1..21: mol_type = other RNA
gtcacacaagtgttatgacat

SEQ ID NO: 122 - RNA, 21 nt, synthetic construct
  source 1..21: mol_type = other RNA
acacaagtgttatgacatcgt

SEQ ID NO: 123 - RNA, 21 nt, synthetic construct
  source 1..21: mol_type = other RNA
cacaagtgttatgacatcgtt

SEQ ID NO: 124 - RNA, 21 nt, synthetic construct
  source 1..21: mol_type = other RNA
acaagtgttatgacatcgttc

SEQ ID NO: 125 - RNA, 21 nt, synthetic construct
  source 1..21: mol_type = other RNA
aagtgttatgacatcgttcca

SEQ ID NO: 126 - RNA, 21 nt, synthetic construct
  source 1..21: mol_type = other RNA
agtgttatgacatcgttccaa

SEQ ID NO: 127 - RNA, 21 nt, synthetic construct
  source 1..21: mol_type = other RNA
gtgttatgacatcgttccaac

SEQ ID NO: 128 - RNA, 21 nt, synthetic construct
  source 1..21: mol_type = other RNA
tgttatgacatcgttccaacc

SEQ ID NO: 129 - RNA, 21 nt, synthetic construct
  source 1..21: mol_type = other RNA
gttatgacatcgttccaacca

SEQ ID NO: 130 - RNA, 21 nt, synthetic construct
  source 1..21: mol_type = other RNA
ttatgacatcgttccaaccag

SEQ ID NO: 131 - RNA, 21 nt, synthetic construct
  source 1..21: mol_type = other RNA
tatgacatcgttccaaccagt

SEQ ID NO: 132 - RNA, 21 nt, synthetic construct
  source 1..21: mol_type = other RNA
atgacatcgttccaaccagtt

SEQ ID NO: 133 - RNA, 21 nt, synthetic construct
  source 1..21: mol_type = other RNA
tgacatcgttccaaccagttc

SEQ ID NO: 134 - RNA, 21 nt, synthetic construct
  source 1..21: mol_type = other RNA
gacatcgttccaaccagttca

SEQ ID NO: 135 - RNA, 21 nt, synthetic construct
  source 1..21: mol_type = other RNA
acatcgttccaaccagttcaa

SEQ ID NO: 136 - RNA, 21 nt, synthetic construct
  source 1..21: mol_type = other RNA
catcgttccaaccagttcaaa

SEQ ID NO: 137 - RNA, 21 nt, synthetic construct
  source 1..21: mol_type = other RNA
atcgttccaaccagttcaaag

SEQ ID NO: 138 - RNA, 21 nt, synthetic construct
  source 1..21: mol_type = other RNA
tcgttccaaccagttcaaagc

SEQ ID NO: 139 - RNA, 21 nt, synthetic construct
  source 1..21: mol_type = other RNA
cgttccaaccagttcaaagct

SEQ ID NO: 140 - RNA, 21 nt, synthetic construct
  source 1..21: mol_type = other RNA
ttccaaccagttcaaagcttg

SEQ ID NO: 141 - RNA, 21 nt, synthetic construct
  source 1..21: mol_type = other RNA
tccaaccagttcaaagcttgt

SEQ ID NO: 142 - RNA, 21 nt, synthetic construct
  source 1..21: mol_type = other RNA
gttattgaccctatcagtggg

SEQ ID NO: 143 - RNA, 21 nt, synthetic construct
  source 1..21: mol_type = other RNA
ttattgaccctatcagtggga

SEQ ID NO: 144 - RNA, 21 nt, synthetic construct
  source 1..21: mol_type = other RNA
tattgaccctatcagtgggaa

SEQ ID NO: 145 - RNA, 21 nt, synthetic construct
  source 1..21: mol_type = other RNA
ccacaaaagaatcctcaagtt

SEQ ID NO: 146 - RNA, 21 nt, synthetic construct
  source 1..21: mol_type = other RNA
aaagaatcctcaagttcctcc

SEQ ID NO: 147 - RNA, 21 nt, synthetic construct
  source 1..21: mol_type = other RNA
tcaagttcctccagcttttta

SEQ ID NO: 148 - RNA, 21 nt, synthetic construct
  source 1..21: mol_type = other RNA
agttcctccagctttttatgt

SEQ ID NO: 149 - RNA, 21 nt, synthetic construct
  source 1..21: mol_type = other RNA
gttcctccagctttttatgtc

SEQ ID NO: 150 - RNA, 21 nt, synthetic construct
  source 1..21: mol_type = other RNA
ttcctccagctttttatgtct

SEQ ID NO: 151 - RNA, 21 nt, synthetic construct
  source 1..21: mol_type = other RNA
ccaaagcctgccttcatgaag

SEQ ID NO: 152 - RNA, 21 nt, synthetic construct
  source 1..21: mol_type = other RNA
gccttcatgaagcagaacctg

SEQ ID NO: 153 - RNA, 21 nt, synthetic construct
  source 1..21: mol_type = other RNA
cttcatgaagcagaacctgga

SEQ ID NO: 154 - RNA, 21 nt, synthetic construct
  source 1..21: mol_type = other RNA
ttcatgaagcagaacctggat

SEQ ID NO: 155 - RNA, 21 nt, synthetic construct
  source 1..21: mol_type = other RNA
gaagcagaacctggatgagct

SEQ ID NO: 156 - RNA, 21 nt, synthetic construct
  source 1..21: mol_type = other RNA
aagcagaacctggatgagctt

SEQ ID NO: 157 - RNA, 21 nt, synthetic construct
  source 1..21: mol_type = other RNA
agcagaacctggatgagcttg

SEQ ID NO: 158 - RNA, 21 nt, synthetic construct
  source 1..21: mol_type = other RNA
gcagaacctggatgagcttgg

SEQ ID NO: 159 - RNA, 21 nt, synthetic construct
  source 1..21: mol_type = other RNA
ggatgagtcaggaaaagttgt

SEQ ID NO: 160 - RNA, 21 nt, synthetic construct
  source 1..21: mol_type = other RNA
gatgagtcaggaaaagttgta

SEQ ID NO: 161 - RNA, 21 nt, synthetic construct
  source 1..21: mol_type = other RNA
atgagtcaggaaaagttgtag

SEQ ID NO: 162 - RNA, 21 nt, synthetic construct
  source 1..21: mol_type = other RNA
agtcaggaaaagttgtagata

SEQ ID NO: 163 - RNA, 21 nt, synthetic construct
  source 1..21: mol_type = other RNA
aaagttgtagatatttattcc

SEQ ID NO: 164 - RNA, 21 nt, synthetic construct
  source 1..21: mol_type = other RNA
aagttgtagatatttattcca

SEQ ID NO: 165 - RNA, 21 nt, synthetic construct
  source 1..21: mol_type = other RNA
agttgtagatatttattccaa

SEQ ID NO: 166 - RNA, 21 nt, synthetic construct
  source 1..21: mol_type = other RNA
tttgatgtaattaatcttgct

SEQ ID NO: 167 - RNA, 21 nt, synthetic construct
  source 1..21: mol_type = other RNA
gtaattaatcttgctgctgag

SEQ ID NO: 168 - RNA, 21 nt, synthetic construct
  source 1..21: mol_type = other RNA
taattaatcttgctgctgaga

SEQ ID NO: 169 - RNA, 21 nt, synthetic construct
  source 1..21: mol_type = other RNA
aattaatcttgctgctgagaa

SEQ ID NO: 170 - RNA, 21 nt, synthetic construct
  source 1..21: mol_type = other RNA
ttaatcttgctgctgagaaaa

SEQ ID NO: 171 - RNA, 21 nt, synthetic construct
  source 1..21: mol_type = other RNA
gcaagccctgatcctcacacc

SEQ ID NO: 172 - RNA, 21 nt, synthetic construct
  source 1..21: mol_type = other RNA
caagccctgatcctcacacca

SEQ ID NO: 173 - RNA, 21 nt, synthetic construct
  source 1..21: mol_type = other RNA
ctgatcctcacaccagcaggt

SEQ ID NO: 174 - RNA, 21 nt, synthetic construct
  source 1..21: mol_type = other RNA
tgaccgccgtgaatgtagacg

SEQ ID NO: 175 - RNA, 21 nt, synthetic construct
  source 1..21: mol_type = other RNA
gaccgccgtgaatgtagacgc

SEQ ID NO: 176 - RNA, 21 nt, synthetic construct
  source 1..21: mol_type = other RNA
accgccgtgaatgtagacgcc

SEQ ID NO: 177 - RNA, 21 nt, synthetic construct
  source 1..21: mol_type = other RNA
ccgccgtgaatgtagacgccc

SEQ ID NO: 178 - RNA, 21 nt, synthetic construct
  source 1..21: mol_type = other RNA
cctcatgaacactggctgcaa

SEQ ID NO: 179 - RNA, 21 nt, synthetic construct
  source 1..21: mol_type = other RNA
tttatgtgcttgaagattcag

SEQ ID NO: 180 - RNA, 21 nt, synthetic construct
  source 1..21: mol_type = other RNA
ttatgtgcttgaagattcagg

SEQ ID NO: 181 - RNA, 21 nt, synthetic construct
  source 1..21: mol_type = other RNA
tatgtgcttgaagattcaggc

SEQ ID NO: 182 - RNA, 21 nt, synthetic construct
  source 1..21: mol_type = other RNA
atgtgcttgaagattcaggct

SEQ ID NO: 183 - RNA, 21 nt, synthetic construct
  source 1..21: mol_type = other RNA
aatgctgtatgtcattaaagt

SEQ ID NO: 184 - RNA, 21 nt, synthetic construct
  source 1..21: mol_type = other RNA
tgctgtatgtcattaaagtgc

SEQ ID NO: 185 - RNA, 21 nt, synthetic construct
  source 1..21: mol_type = other RNA
gtatgtcattaaagtgcactg

SEQ ID NO: 186 - RNA, 21 nt, synthetic construct
  source 1..21: mol_type = other RNA
tatgtcattaaagtgcactgt

SEQ ID NO: 187 - RNA, 21 nt, synthetic construct
  source 1..21: mol_type = other RNA
atgtcattaaagtgcactgtg

SEQ ID NO: 188 - RNA, 21 nt, synthetic construct
  source 1..21: mol_type = other RNA
tgtcattaaagtgcactgtgt

SEQ ID NO: 189 - RNA, 21 nt, synthetic construct
  source 1..21: mol_type = other RNA
gtcattaaagtgcactgtgtc

SEQ ID NO: 190 - RNA, 21 nt, synthetic construct
  source 1..21: mol_type = other RNA
tcattaaagtgcactgtgtcc

SEQ ID NO: 191 - RNA, 21 nt, synthetic construct
  source 1..21: mol_type = other RNA
cattaaagtgcactgtgtcct

SEQ ID NO: 192 - RNA, 21 nt, synthetic construct
  source 1..21: mol_type = other RNA
attaaagtgcactgtgtcctg

SEQ ID NO: 193 - RNA, 21 nt, synthetic construct
  source 1..21: mol_type = other RNA
ttaaagtgcactgtgtcctga

SEQ ID NO: 194 - RNA, 21 nt, synthetic construct
  source 1..21: mol_type = other RNA
tgctctgaaaatgttgacact

SEQ ID NO: 195 - RNA, 19 nt, synthetic construct
  source 1..19: mol_type = other RNA
ccgcggaagccggcatgct

SEQ ID NO: 196 - RNA, 19 nt, synthetic construct
  source 1..19: mol_type = other RNA
cgcggaagccggcatgctg

SEQ ID NO: 197 - RNA, 19 nt, synthetic construct
  source 1..19: mol_type = other RNA
gcggaagccggcatgctgg

SEQ ID NO: 198 - RNA, 19 nt, synthetic construct
  source 1..19: mol_type = other RNA
ttatgacatcgttccaacc

SEQ ID NO: 199 - RNA, 19 nt, synthetic construct
  source 1..19: mol_type = other RNA
atgacatcgttccaaccag

SEQ ID NO: 200 - RNA, 19 nt, synthetic construct
  source 1..19: mol_type = other RNA
tgacatcgttccaaccagt

SEQ ID NO: 201 - RNA, 19 nt, synthetic construct
  source 1..19: mol_type = other RNA
gacatcgttccaaccagtt

SEQ ID NO: 202 - RNA, 19 nt, synthetic construct
  source 1..19: mol_type = other RNA
acatcgttccaaccagttc

SEQ ID NO: 203 - RNA, 19 nt, synthetic construct
  source 1..19: mol_type = other RNA
accgccgtgaatgtagacg

SEQ ID NO: 204 - RNA, 19 nt, synthetic construct
  source 1..19: mol_type = other RNA
ccgccgtgaatgtagacgc

SEQ ID NO: 205 - protein, 245 aa, Homo sapiens
MEWSWVFLFFLSVTTGVHSQVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCG

SEQ ID NO: 206 - RNA, 735 nt, Homo sapiens
  source 1..735: mol_type = mRNA
atggaatggtcatgggtttttttgtttttcctctcagttacgactggtgtccatagccaagtccaactggtgcagtccggtgcggaggttaagaagcccggagcgagcgtaaaggtgagttgtaaagcgagtggatacacgttcacgaactattggatgcattgggttcgacaagcaccgggtcagggacttgagtggatgggagaaattaatccgattaacggtcgcagtaactatgcgcagaaattccaaggccgagtaactctcaccgtggacacgtccatctctacagcgtacatggaactcagcaggttgcgctctgacgataccgcagtttattattgcgcgcgagggacgcgggctatgcactattgggggcagggcaccctcgtcaccgtatcatctgcgagtacgaagggaccttctgtgttcccattggctcccagcagcaaaagtaccagtggtggaacagctgcgcttggatgcctggtgaaagattatttccccgagccggtgacagtcagctggaacagcggcgcactcaccagcggtgtacatacgttcccggcggttttgcaatctagtggcctctattcccttagttccgtagttaccgtcccatcttcaagcctcggaacccagacttacatctgcaacgtcaatcataagcccagtaacacaaaagttgataagagagtagagccgaaatcctgtgataagacccacacatgtggg

SEQ ID NO: 207 - protein, 234 aa, Homo sapiens
MSVPTQVLGLLLLWLTDARCDIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - RNA, 747 nt, Homo sapiens
  source 1..747: mol_type = mRNA
gcctccggactctagagccgccaccatgagcgtaccaacccaggtgctcggactcctgttgttgtggctcaccgatgcaagatgcgatatacaaatgacacaaagcccaagtagtttgtcagccagcgtaggggatagagttactataacttgccgaacgtctgaaaatatatataataacctcgcgtggtaccagcagaagcccggcaaggcccctaaactcctcatttatgcagctactaacctcgctgaaggagtaccatcaaggttctcaggcagcgggtctggaactgactacacattgactatttcaagccttcagccagaggacttcgctacatactactgtcaacacttctgggggactccgcttactttcggaggcggtaccaaagtggagataaaacggacggttgctgctccgagcgtttttatattcccgccctctgatgaacagctgaaatcaggcactgcgagcgttgtttgcttgctgaataacttttacccccgcgaggcgaaagtacaatggaaggtagacaacgcactgcaatctgggaatagtcaagagagtgttaccgaacaagattcaaaagattccacttattcccttagttctactttgacactgagcaaagcagattacgagaaacataaggtctacgcctgcgaggtgacgcaccagggcctgagcagcccagttacaaagtccttcaatcgaggtgagtgttaggcggccgctataagggt

SEQ ID NO: 209 - RNA, 23 nt, synthetic construct
  source 1..23: mol_type = other RNA
  modified_base 1: mod_base = OTHER; note = 2'-O-methyl modified nucleotide
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tacaacttttcctgactcatctt

SEQ ID NO: 210 - RNA, 23 nt, synthetic construct
  source 1..23: mol_type = other RNA
  modified_base 1: mod_base = OTHER; note = 5'Vinyl phosphonate unlocked nucleobase analog to uridine
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tacaacttttcctgactcatctt

SEQ ID NO: 211 - RNA, 23 nt, synthetic construct
  source 1..23: mol_type = other RNA
  modified_base 1: mod_base = OTHER; note = 2'-O-methyl modified nucleotide
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tgaataaatatctacaacttttt

SEQ ID NO: 212 - RNA, 23 nt, synthetic construct
  source 1..23: mol_type = other RNA
  modified_base 1: mod_base = OTHER; note = 5'Vinyl phosphonate unlocked nucleobase analog to uridine
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tgaataaatatctacaacttttt

SEQ ID NO: 213 - RNA, 23 nt, synthetic construct
  source 1..23: mol_type = other RNA
  modified_base 1: mod_base = OTHER; note = 2'-O-methyl modified nucleotide
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tggaataaatatctacaactttt

SEQ ID NO: 214 - RNA, 23 nt, synthetic construct
  source 1..23: mol_type = other RNA
  modified_base 1: mod_base = OTHER; note = 5'Vinyl phosphonate unlocked nucleobase analog to uridine
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tggaataaatatctacaactttt

SEQ ID NO: 215 - RNA, 23 nt, synthetic construct
  source 1..23: mol_type = other RNA
  modified_base 1: mod_base = OTHER; note = 2'-O-methyl modified nucleotide
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tgcaagattaattacatcaaatt

SEQ ID NO: 216 - RNA, 23 nt, synthetic construct
  source 1..23: mol_type = other RNA
  modified_base 1: mod_base = OTHER; note = 5'Vinyl phosphonate unlocked nucleobase analog to uridine
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
tgcaagattaattacatcaaatt

SEQ ID NO: 217 - RNA, 23 nt, synthetic construct
  source 1..23: mol_type = other RNA
  modified_base 1: mod_base = OTHER; note = 2'-O-methyl modified nucleotide
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
ttgaatcttcaagcacataaatt

SEQ ID NO: 218 - RNA, 23 nt, synthetic construct
  source 1..23: mol_type = other RNA
  modified_base 1: mod_base = OTHER; note = 5'Vinyl phosphonate unlocked nucleobase analog to uridine
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..23: mod_base = OTHER; note = 2'-O-methyl modified nucleotide
  modified_base 1..3: mod_base = OTHER; note = phosphorothioate internucleotide linkage
  modified_base 21..23: mod_base = OTHER; note = phosphorothioate internucleotide linkage
ttgaatcttcaagcacataaatt

SEQ ID NO: 219 - RNA, 21 nt, synthetic construct
  source 1..21: mol_type = other RNA
  modified_base 1: mod_base = OTHER; note = 2'-O-methyl modified nucleotide
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
tctggttggaacgatgtcatt

SEQ ID NO: 220 - RNA, 21 nt, synthetic construct
  source 1..21: mol_type = other RNA
  modified_base 1: mod_base = OTHER; note = 5'Vinyl phosphonate unlocked nucleobase analog to uridine
  modified_base 2: mod_base = OTHER; note = 2'fluoro modified nucleotide
  modified_base 3..5: mod_base = OTHER; note = 2'-O-methyl modified nucleotide
  modified_base 6: mod_base = OTHER; note = 2'fluoro modified nucleotide
  modified_base 7..13: mod_base = OTHER; note = 2'-O-methyl modified nucleotide
  modified_base 14: mod_base = OTHER; note = 2'fluoro modified nucleotide
  modified_base 15: mod_base = OTHER; note = 2'-O-methyl modified nucleotide
  modified_base 16: mod_base = OTHER; note = 2'fluoro modified nucleotide
  modified_base 17..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
tctggttggaacgatgtcatt

SEQ ID NO: 221 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
gatgagtcaggaaaagttgta

SEQ ID NO: 222 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
gatgagtcaggaaaagttgta

SEQ ID NO: 223 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
aaagttgtagatatttattca

SEQ ID NO: 224 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
aaagttgtagatatttattca

SEQ ID NO: 225 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
aagttgtagatatttattcca

SEQ ID NO: 226 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
aagttgtagatatttattcca

SEQ ID NO: 227 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
tttgatgtaattaatcttgca

SEQ ID NO: 228 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
tttgatgtaattaatcttgca

SEQ ID NO: 229 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
tttatgtgcttgaagattcaa

SEQ ID NO: 230 - RNA, 21 nt, synthetic construct
  source 1..21: mol_type = other RNA
  modified_base 1..8: mod_base = OTHER; note = 2'-O-methyl modified nucleotide
  modified_base 9..11: mod_base = OTHER; note = 2'fluoro modified nucleotide
  modified_base 12..21: mod_base = OTHER; note = 2'-O-methyl modified nucleotide
  modified_base 1..3: mod_base = OTHER; note = phosphorothioate internucleotide linkage
  modified_base 19..21: mod_base = OTHER; note = phosphorothioate internucleotide linkage
tttatgtgcttgaagattcaa

SEQ ID NO: 231 - RNA, 19 nt, synthetic construct
  source 1..19: mol_type = other RNA
  modified_base 1..6: mod_base = OTHER; note = 2'-O-methyl modified nucleotide
  modified_base 7..9: mod_base = OTHER; note = 2'fluoro modified nucleotide
  modified_base 10..19: mod_base = OTHER; note = 2'-O-methyl modified nucleotide
  modified_base 1..3: mod_base = OTHER; note = phosphorothioate internucleotide linkage
  modified_base 17..19: mod_base = OTHER; note = phosphorothioate internucleotide linkage
tgacatcgttccaaccaga

SEQ ID NO: 232 - RNA, 19 nt, synthetic construct
  source 1..19: mol_type = other RNA
  modified_base 1..6: mod_base = OTHER; note = 2'-O-methyl modified nucleotide
  modified_base 7..9: mod_base = OTHER; note = 2'fluoro modified nucleotide
  modified_base 10..19: mod_base = OTHER; note = 2'-O-methyl modified nucleotide
  modified_base 1..3: mod_base = OTHER; note = phosphorothioate internucleotide linkage
  modified_base 17..19: mod_base = OTHER; note = phosphorothioate internucleotide linkage
tgacatcgttccaaccaga

SEQ ID NO: 233-236 - skipped (4 entries)

SEQ ID NO: 237 - RNA, 3279 nt, Homo sapiens
  source 1..3279: mol_type = mRNA
attctgcggccgaggattacatttatgcacgaacgggcttactggttccagattccccacttgggcacaggcataggaggcttgttttccaaattgctggttttaattgcacctgcctttcagattacctctgggaatctgtgggaggagccgagagggtggaaaatgtttcttagctttgcaaaaggaagaaaactttgtcacccagcgggagacctcagccacgagtaacccggggagacaccagaaccgggacgggctttgactgatttgcctacgagggttccgtaggaaaggacgcttgaattcggcgcttcggcggcggcggcggccgcgcgagttccctgctcaccctccctctccgcggaagtccccacgaggtggcttcagggtgtaacagagcgcgcggctccagtccgaaggcagcggccgggggagggaaggaggggaccgaacccccgaggagtttcgcagaatcaacttctggttagagttatgggaagcgcggttatggacaccaagaagaaaaaagatgtttccagccccggcgggagcggcggcaagaaaaatgccagccagaagaggcgttcgctgcgcgtgcacattccggacctgagctccttcgccatgccgctcctggacggagacctggagggttccggaaagcattcctctcgaaaggtggacagccccttcggcccgggcagcccctccaaagggttcttctccagaggcccccagccccggccctccagccccatgtctgcacctgtgaggcccaagaccagccccggctctcccaaaaccgtgttcccgttctcctaccaggagtccccgccacgctcccctcgacgcatgagcttcagtgggatcttccgctcctcctccaaagagtcttcccccaactccaaccctgctacctcgcccgggggcatcaggtttttctcccgctccagaaaaacctccggcctctcctcctctccgtcaacacccacccaagtgaccaagcagcacacgtttcccctggaatcctataagcacgagcctgaacggttagagaatcgcatctatgcctcgtcttcccccccggacacagggcagaggttctgcccgtcttccttccagagcccgaccaggcctccactggcatcaccgacacactatgctccctccaaagccgcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtccgagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggagtgaccgccgtgaatgtagacgccctaggaggagaacttgaacaaagtctctgggtcacgttttgcctcatgaacactggctgcaagtggttaagaatgtatatcagggtttaacaataggtatttcttccagtgatgttgaaattaagcttaaaaaagaaagattttatgtgcttgaagattcaggcttgcattaaaagactgttttcagacctttgtctgaaggattttaaatgctgtatgtcattaaagtgcactgtgtcctgaagttttcattatttttcatttcaaagaattcactggtatggaacaggtgatgtggcataaggtgagtgcacggtatgttcagatcacagtgccttatgtccgaatacagcaatatgtcaccgccgcagccggggcgcacgcgtgtgaaacaacaccgagcttgaatgtggaagtctttgaaccttttaccaaatcagtttgttttctttagatttgtcaaaaagttgtaatttgaatataaataattactttaaaattgtaatgacacttttacacgtaagtgttttgttctgggctaccgtgtcaacgaggctgctttacaacagctttatttatttttactttcatgcaatttttttacacatcttttggtggagtaaacttcaccacatccatgaataaactctcagttattttgaaatggcaaatttctcattatttaagtttggatctggaaaggacatgacttctgaaatagccgctgctgggttttaaaagctgaggtctctcaaagtgtggaggagacgttgccgtcaggcgggagccaagtgccgggaagatgtctattttttttcttgtgtattgaaatgtaaaatcatgatgtttgttatgactgctgatgcgattgtttttgtaaattttattgtggcatatacagtattgtcatacagttgaagagaaacaatgtttcctaatgtaagtgctctgaaaatgttgacactgtatatatatatatgaggatagtttgttttttttttgttttgggtttttttttttcagattgaaaaattaaaatagatcctactatcta

SEQ ID NO: 238 - RNA, 3340 nt, Mus musculus
  source 1..3340: mol_type = mRNA
ccctgtcgctttattgtgcggcccagggttatgcatttctctaccaagtgtctctggccaccgtttccatcgaggcacagacacgagaaggctgttttccaaattgctgttgttaattgcacctgcctggccgactacctctgggaatttgtgggaggagccgagaggtggaaaatgttgcttcgctttgcagaaggaggaaaactttatcacccagcacaagacttctgccacgcgtaaccaaaagtagcacgggccgagactctgcacccaatgattccgtaggagaaagaggacgctccaacccggcattccgcggactgccagcccctcgggtggagccccgcgtgttacctgcgccacccgccctccccgcggaagtccccacgaggtgttttcagggtgggaggacagtcataaaaagcgcgtggctccagtgtaacggcagcagtcctgggaggcagcgaggggaccgaaccccggggacgaggcaccggaattaacttctagctaaagttatgggaagcgcagccatggacaccaagaagaagaaagaggtttccagcccgggcgggagcagtggcaaaaagaaccctagcctgaagaggcggtcactgcgagtgcacattccggacctgagttcctttgccatgccgctcctggatggagatgtggagaattcagaaaagcattcatctcggaaggtggacagtcccttcagctcaggcagcccctccagaggactcttctccagaggcccccagccccggccctccagccctgtgtctgctcccgtgaggcccaagacgagccctggctctcccaaaactgtgttcccgttctcctaccaggagtcccctccacgctctccacgccggatgagcttcagtgggatcttccgctcctcatccaaagagtcttcgcccaactccaacccgtctacctctcctgggggcatccggttcttctcacggtccagaaaaacctccagcgtctcctcttctccgtcaacacccacccaagtgaccaagcagcacccatttcccctggagtcctacaagcaggagcctgaacgcccagagagccgcatctatgcttcctcgtcccctccagacactgggcagcgcttctgcctggcctttcagagcccagcgcgacctccgctggcatccccgacataccatgctcctttgagaactgcggtgctggcggcggccccgggacccgcggaagccggcatgctggagaagctggagttccaggaggaagaagactcagaaagtggcgtttatatgcgattcatgaggtcacacaagtgttatgacatcgttccaaccagttcaaagcttgttgtcttcgacactacgttgcaagtcaaaaaggccttctttgccttggtagccaacggagtccgtgcagcgccgctgtgggaaagtaaaaagcagagctttgtaggaatgctcacgattacagatttcataaatatcctacacagatactataaatcacccatggtacagatttatgaattggaagaacataagattgaaacgtggagggagttgtacttacaagaaaccttcaagcctttggtgaacatctctccagatgcgagcctcttcgatgctgtatactcgttgatcaaaaataaaatccacagattgccagttattgaccctatcagtgggaacgcactttatatacttacccacaaaagaatcctcaagttcctccagctttttatgtctgacatgccaaagcctgccttcatgaagcagaacctggatgagcttggaatcggaacgtaccacaatattgccttcattcacccagacactcccatcatcaaagccttgaacatcttcgtggagagacggatatccgcattgcctgtggtggatgagtcaggaaaagttgtagatatttattccaagtttgatgtaattaatcttgctgctgagaaaacgtacaataacttggacatcacagtgacccaggccctgcagcaccgctcgcagtattttgagggtgtggtgaagtgcagtaagctggaaacactggagaccatcgtggacaggatagtgagagccgaggtccatcggctggttgtagtgaatgaagcagatagcattgtgggtattatctccctgtcagacatcctgcaagccctgatcctcacaccagcaggtgccaaacagaaggagacagagactgaatgaccgccgtgaatgtagacgcccgcgcaggagaacttgaacaaagtctcggggtcacggtttgcctcatgaacactggctgcaatagaagagagtaaaatggctaagaatgtataccagggtttagcgatgggtatttttccagtgatttgtaagtataggaaaagattttatgtgcttgaagattcaggcttgcgttcaaagactgccctcagacctgtctgaaggattttcaatgctgtatgtcattaaagtgcactgtgtcctgaaatagtactacttttcatttcaaagaagtcactgggatggaacaggtgacagaaagtgagtgaacggcacgtccagatcacggtgccttatgtccaaacgcaatatgtcaccgccgctgccgcacgccccaaagtgctgctgaagagacacccagcccaagtgtgagcatcttcagaccttttacccagtctcttccttttcacttaattggttgaaaagttttaatttgagtacaggcaactgaaaatgtgatgtgatgcctctaagcgctctgcatccgtcctgttctggctgccgtgttagcgaggccggcttggcaacagcgctttatttatttttactctcatgcaggtttcaacctacatcctttggtgagtagacgtcaccatacctacgaataaaaccttgattgttctctcaagtggaaaatctctggagattttagtttggatctggaaaggctatgccttccaaacagccactgtagggtcttctaaacgtttatgaaaacaggctgtggtcgcagggaagaagtaacggaagacgtctttttttttattgtgtgttgaaatgtaaactcatgatgtttgtgtgaccgctgacaagacgcttgtaacttttattgtgacacataaaacagtattgtcatgcagttgaagggaaactctttcccctcccacaccccacaacccccacagcccccagtgtcagtgctctgaaaatgttgacactatatatctgtgtgtgtatatatgagcagagtttgggggtttgttttggggttttgtttcgttttgtttttcagattgaaaattaaaataaatcctgtctaccagtttgctaaagtttttatg

SEQ ID NO: 239 - RNA, 3263 nt, Macaca fascicularis
  source 1..3263: mol_type = mRNA
attctgcggccgaggattacatttatgcacgaacggtctcgctggctccagattccccacttgggcacaggctaggagggttgttttccaaattgctggttttaattgcacctgcctttcagattacctctgggaatctgtgggaggagccgagagggtggaaaatgtttcttctctttgcaaaaggaagaaaactttgtcacccagcgggagacctcagccacgcgtaacccgcgagacaccagaaccgggacgggctttgactgattctgcctgcgagagttccgtaggaaaggacgcttgaactcggcgctccggcggcggcggcacgagttccctgctcactctccctccccgcggaagtccccacgaggtggcttcggggtgtaacagagcgcgcggctccagtccgacggcagcggccggggcagggaaggaggggaccgaacccccgaggagttacccagaatcaacttctggttagagttatgggaagcgcggttatggacaccaagaagaaaaaagatgtttccagccccggcgggagcggcggcaagaaaaatgccagccagaagaggcgttctctgcgcgtgcacattccggacctgagctcctttgccatgccgctcctggacggagacctggagggttccggaaagcattcctctcgaaaggtggacagccccttcggcccgggcagcccctccaaagggttcttctccagaggcccccagccccggccctccagccccatgtctgcacccgtgaggcccaagaccagccccggctctccgaaaaccgtgttcccgttctcctaccaggagtccccgccgcgctcccctcgacgcatgagcttcagcgggatcttccgctcctcctccaaagagtcttcccccaactccaaccctgctacctcgcccgggggcatcaggtttttctcccgctccagaaaaacctccggcctctcctcctctccgtcaacacccacccaagtgaccaagcagcacacatttcccctggaatcctataagcacgagcctgaacggttagagaatcgcatctatgcctcgtcttcccctccggacacagggcagaggttctgcccgtcttccttccagagcccgaccaggcctccactggcatcaccgacacactatgctccctccaaagccgcggcgctggcggcggccccgggacccgcggaagccggcatgctggagaagctggagttcgaggacgaagcagaagactcagaaagtggtgtttacatgcgattcatgaggtcacacaagtgttatgacatcgttccaaccagttcaaagcttgtcgtctttgatactacattacaagttaaaaaggccttctttgctttggtagccaacggtgtccgagcagcaccactgtgggagagtaaaaaacaaagttttgtaggaatgctaacaattacagatttcataaatatactacatagatactataaatcgcctatggtacagatttatgaattagaggaacataaaattgaaacctggagggagctttatttacaagaaacatttaagcctttagtgaatatatctccagatgcaagcctcttcgatgctgtatactccttgatcaaaaataaaatccacagattgccagttattgaccctatcagtgggaatgcactttatatactcacccacaaaagaatcctcaagttcctccagctttttatgtctgatatgccaaagcctgccttcatgaagcagaacctggatgagcttggaataggaacgtaccacaacattgccttcatacatccagacactcccatcattaaagccttgaacatatttgtggaaagacgaatatcagctctgcctgttgtggatgagtcaggaaaagttgtagatatttattccaaatttgatgtaattaatcttgctgctgagaaaacatacaataacctagatatcacagtgacccaggcccttcagcaccgttcacagtattttgaaggtgttgtaaagtgcaataagctggaaatactggagaccattgtggacagaatagtgagagctgaggtccatcggctggtggtggtaaatgaagcagatagtatcgtgggtattatttccctgtcggatattctgcaagccctgatcctcacaccagcaggtgccaaacaaaaggagacggaaacagagtgaccgccgtgaatgtagacgccctaggaggagaacttgaacaaagtttctgggtcacgttttgcctcatgaacactggctgcaagtggttaagaatgtatatcagggtttaatgatgggtatttcttccagtgatgttggaattaagcttaaaaaagaaagattttatgtgcttgaagattcaggcttgcattaaaagactgttttcagacctttgtctgacggattttaaatgctgtatgtcattaaagtgcactgtgtcctgaagttttcattatttttcatttcaaagagttcactggtatggaacaggtgatgcgacataaggtgagtgcacggcatgttcagatcacagtgccttatgtccgaatacagcaatatgtcacctccgcagccggggcgcacgcgtgcgaagccacaccgagcttgaatgtggaagtctttgaaccttttaccaaatcagtttgttttcattagatttgtcaaaaagttgtcatttgaatataacgaatgactttaaaactgtaatgacacttttacacgtgtttcgttccgggctaccgtgtcaacaaggctgctttacaacagctttatttagttttactttcatgccatttttttacacatcttttggtggggtaaacttcaccacacccatgaataaactctcagttattttgaaatggcaaatttctcattatttaagtttggatctggaaaggacatgacttctgaaacagccgctgctgggttttcaaagctgaggtctcaaacgtgtaggagacgttgccgtcaggcgggagccaagtgccgggaagatgtctattttttttcttgtgtattaaaatgtaaaatcatgatgtttgtatgactgccgatgcgattgtgtttgtaaattttattgtggcatatacagtattgtcatacagttgaagagaaacaatgtttcctaatgtaagtgctctgaaaatgttgacactgtatatatatatatgaggatagtttgggttttttttgtttttgggtttttttttcagattgaaaaattaaaataaatcctactatctacca

SEQ ID NO: 240 - protein, 9 aa, synthetic construct
YTFTNYWMH

SEQ ID NO: 241 - protein, 17 aa, synthetic construct
  VARIANT 6: note = N or Q
  VARIANT 13: note = Q or E
EINPIXGRSNYAXKFQG

SEQ ID NO: 242 - protein, 17 aa, synthetic construct
EINPINGRSNYAQKFQG

SEQ ID NO: 243 - protein, 17 aa, synthetic construct
EINPINGRSNYAEKFQG

SEQ ID NO: 244 - protein, 17 aa, synthetic construct
EINPIQGRSNYAEKFQG

SEQ ID NO: 245 - protein, 7 aa, synthetic construct
GTRAMHY

SEQ ID NO: 246 - protein, 11 aa, synthetic construct
  VARIANT 8: note = N or S
RTSENIYXNLA

SEQ ID NO: 247 - protein, 11 aa, synthetic construct
RTSENIYNNLA

SEQ ID NO: 248 - protein, 11 aa, synthetic construct
RTSENIYSNLA

SEQ ID NO: 249 - protein, 7 aa, synthetic construct
  VARIANT 2: note = A or G
  VARIANT 7: note = D or E
AXTNLAX

SEQ ID NO: 250 - protein, 7 aa, synthetic construct
AATNLAD

SEQ ID NO: 251 - protein, 7 aa, synthetic construct
AATNLAE

SEQ ID NO: 252 - protein, 7 aa, synthetic construct
AGTNLAD

SEQ ID NO: 253 - protein, 10 aa, synthetic construct
  VARIANT 10: note = F or absent
QHFWGTPLTX

SEQ ID NO: 254 - protein, 9 aa, synthetic construct
QHFWGTPLT

SEQ ID NO: 255 - protein, 10 aa, synthetic construct
QHFWGTPLTF

SEQ ID NO: 256 - protein, 116 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 257 - protein, 116 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 258 - protein, 116 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 259 - protein, 116 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 260 - protein, 116 aa, synthetic construct
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 261 - protein, 107 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 262 - protein, 107 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 263 - protein, 107 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 264 - protein, 107 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

SEQ ID NO: 265 - protein, 107 aa, synthetic construct
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 266-269 - skipped (4 entries)

SEQ ID NO: 270 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 271 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 272 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 274 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 275 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 276 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 277 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 278 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 279 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 280 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 281 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 282 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 283 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 284 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 285 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 286 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 287 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 288 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 289 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 290 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 291 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 292 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 293 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 294 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 214 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 214 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - protein, 214 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 24 aa, synthetic construct
CGIFGEIEELIEEGLENLIDWGNA

SEQ ID NO: 299 - protein, 24 aa, synthetic construct
GLFEAIEGFIENGWEGMIDGWYGC

SEQ ID NO: 300 - protein, 27 aa, synthetic construct
GLFEAIEGFIENGWEGMIWDYGSGSCG

SEQ ID NO: 301 - protein, 23 aa, synthetic construct
GLFEAIEGFIENGWEGMIDGWYG

SEQ ID NO: 302 - protein, 27 aa, synthetic construct
GLFEAIEGFIENGWEGMIWDYGSGSCK

SEQ ID NO: 303 - protein, 27 aa, synthetic construct
CLIGAILKVLATGLPTLISWIKNKRKQ

SEQ ID NO: 304 - protein, 26 aa, synthetic construct
GIGAVLKVLTTGLPALISWIKRKRQQ

SEQ ID NO: 305 - protein, 13 aa, synthetic construct
IFGAIAGLLKNIF

SEQ ID NO: 306 - protein, 18 aa, synthetic construct
FFGHLFKLATKIIPSLFQ

SEQ ID NO: 307 - protein, 21 aa, synthetic construct
KETWWETWWTEWSQPKKKRKV

SEQ ID NO: 308 - protein, 19 aa, synthetic construct
LLIILRRRRIRKQAHAHSK

SEQ ID NO: 309 - protein, 26 aa, synthetic construct
DPKGDPKGVTVTVTVTVTGKGDPKPD

SEQ ID NO: 310 - protein, 17 aa, synthetic construct
CSIPPEVKFNKPFVYLI

SEQ ID NO: 311 - protein, 27 aa, synthetic construct
GWTLNSAGYLLGKINLKALAALAKKIL

SEQ ID NO: 312 - protein, 21 aa, synthetic construct
AGYLLGKINLKALAALAKKIL

SEQ ID NO: 313 - protein, 17 aa, synthetic construct
GALFLGFLGAAGSTMGA

SEQ ID NO: 314 - protein, 20 aa, synthetic construct
HGLASTLTRWAHYNALIRAF

SEQ ID NO: 315 - protein, 20 aa, synthetic construct
GLWRALWRLLRSLWRLLWRA

SEQ ID NO: 316 - protein, 30 aa, synthetic construct
WEAALAEALAEALAEHLAEALAEALEALAA

SEQ ID NO: 317 - protein, 24 aa, synthetic construct
GLFEAIEGFIENGWEGMIDGWYGC

SEQ ID NO: 318 - protein, 23 aa, synthetic construct
GLFGAIAGFIENGWEGMIDGWYG

SEQ ID NO: 319 - protein, 36 aa, synthetic construct
GLFGAIAGFIENGWEGMIDGRQIKIWFQNRRMKWKK

SEQ ID NO: 320 - protein, 26 aa, synthetic construct
GLFGAIAGFIENGWEGMIDGSSKKKK

SEQ ID NO: 321 - protein, 24 aa, synthetic construct
GLFEAIAGFIENGWEGMIDGGGYC

SEQ ID NO: 322 - protein, 23 aa, synthetic construct
GLFHAIAHFIHGGWHGLIHGWYG

SEQ ID NO: 323 - protein, 30 aa, synthetic construct
GLFEAIEGFIENGWEGLAEALAEALEALAA

SEQ ID NO: 324 - protein, 29 aa, synthetic construct
KWKLFKKIGAVLKVLTTGYGRKKRRQRRR

SEQ ID NO: 325 - protein, 16 aa, synthetic construct
RQIKIWFQNRRMKWKK

SEQ ID NO: 326 - protein, 13 aa, synthetic construct
GRKKRRQRRRPPQ

SEQ ID NO: 327 - protein, 18 aa, synthetic construct
LLIILRRRIRKQAHAHSK

SEQ ID NO: 328 - protein, 27 aa, synthetic construct
GWTLNSAGYLLGKINLKALAALAKKIL

SEQ ID NO: 329 - protein, 27 aa, synthetic construct
GALFLGFLGAAGSTMGAWSQPKKKRKV

SEQ ID NO: 330 - protein, 21 aa, synthetic construct
KETWWETWWTEWSQPKKKRKV

SEQ ID NO: 331 - protein, 12 aa, synthetic construct
  VARIANT 7..12: note = Residues may be deleted
RRRRRRRRRRRR

SEQ ID NO: 332 - protein, 18 aa, synthetic construct
KLALKLALKALKAALKLA

SEQ ID NO: 333 - protein, 9 aa, synthetic construct
RRWWRRWRR

SEQ ID NO: 334 - protein, 13 aa, synthetic construct
CGYGPKKKRKVGG

SEQ ID NO: 335 - protein, 8 aa, synthetic construct
KKKKKKKK

SEQ ID NO: 336 - protein, 22 aa, synthetic construct
MVRRFLVTLRIRRACGPPRVRV

SEQ ID NO: 337 - protein, 28 aa, synthetic construct
LSTAADMQGVVTDGMASGLDKDYLKPDD

SEQ ID NO: 338 - protein, 4 aa, synthetic construct
GGFG

SEQ ID NO: 339 - protein, 4 aa, synthetic construct
ALAL

SEQ ID NO: 340 - protein, 4 aa, synthetic construct
GFLG

(19) United States
(12) Patent Application Publication
(10) Pub. No.: US 2026/0191980 A1
(43) Pub. Date: Jul. 9, 2026

(54) METHODS OF TREATING CARDIOMYOPATHY USING PRKAG2-TARGETING ANTIBODY-OLIGONUCLEOTIDE CONJUGATES

(71) Applicant: Atrium Therapeutics, Inc., San Diego, CA (US)

(72) Inventors: Maria Azzurra MISSINATO, San Diego, CA (US); Georgios KARAMANLIDIS, Thousand Oaks, CA (US); Kellie Ann LEMOINE, San Diego, CA (US); Sami Abdulwahab ABDULKADIR, San Diego, CA (US); Subbarao NALLAGATLA, San Diego, CA (US); Maryam JORDAN, Escondido, CA (US); Isaac MARKS, San Diego, CA (US)

(21) Appl. No.: 19/383,467

(22) Filed: Nov. 7, 2025

Related U.S. Application Data
(60) Provisional application No. 63/718,513, filed on Nov. 8, 2024.
(60) Provisional application No. 63/855,074, filed on Jul. 31, 2025.

(51) Int. Cl. A61K 47/68; A61K 9/00

(52) U.S. Cl. CPC A61K 47/6807; A61K 9/0019; A61K 47/6849

(57) ABSTRACT

Disclosed herein are methods of treating cardiomyopathy or modulating PRKAG2 expression or activity in the cardiac muscle tissues using pharmaceutical compositions, of PRKAG2 antibody oligonucleotide conjugates (AOC).

### CROSS-REFERENCE

This application claims the benefit of U.S. Provisional Application No. 63/718,513, filed Nov. 8, 2024, and U.S. Provisional Application No. 63/855,074, filed Jul. 31, 2025, each of which is incorporated herein by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Dec. 2, 2025, is named 45532-792_201_SL.xml and is 427,426 bytes in size.

### BACKGROUND OF THE DISCLOSURE

Gene suppression by RNA-induced gene silencing provides several levels of control: transcription inactivation, small interfering RNA (siRNA)-induced mRNA degradation, and siRNA-induced transcriptional attenuation. In some instances, RNA interference (RNAi) provides long lasting effects over multiple cell divisions. As such, RNAi represents a viable method useful for drug target validation, gene function analysis, pathway analysis, and disease therapeutics.

AMP-activated protein kinase (AMPK) is an energy sensor kinase, composed of 3 subunits: a catalytic subunit (α1 or α2) and 2 regulatory subunits (β1 or β2 subunit and γ1, γ2, or γ3 subunit). The Protein Kinase AMP-Activated Non-Catalytic Subunit Gamma 2 (PRKAG2) gene encodes the γ2 regulatory subunit of the AMPK, which responds to AMP/ATP fluctuations. PRKAG2 is predominantly a cardiac isoform and the human PRKAG2 gene shares high homology (~90%) with the non-human primate and murine PRKAG2 gene. Binding of AMP to the γ2 regulatory subunit encoded by PRKAG2 activates AMPK and induces its conformational changes. As such, mutations in PRKAG2 result in decreased affinity to ATP to so maintain AMPK inactive such that the AMPK loses its ability to sense AMP and ATP levels. Elevated AMPK activity promotes glucose transporter 4 (GLUT4) shuttling to the plasma membrane and increases glucose uptake and intracellular glucose 6-phosphate (G6P) concentration. This leads to an allosteric activation of glycogen synthase (GS), which overrides the inhibitory effect of AMPK on GS, resulting in a net increase in GS activity and excess glycogen storage in muscle cells.

PRKAG2 cardiac syndrome is an autosomal dominant metabolic heart disease characterized by left ventricular hypertrophy (LVH), progressive conduction abnormalities, and ventricular pre-excitation. PRKAG2 cardiac syndrome causes cardiac hypertrophy and electrophysiologic abnormalities, particularly preexcitation (Wolff-Parkinson-White syndrome) and atrioventricular conduction block, glycogen storage disease of the heart. The prevalence of PRKAG2 syndrome is 0.23-1% in patients with suspected HCM. The glycogen accumulation is often associated with an eccentric pattern of hypertrophy and conduction abnormalities that characterize the PRKAG2 cardiac syndrome. Cardiomyopathies caused by glycogen storage diseases including PRKAG2 mutations are distinguished from other types of hypertrophic cardiomyocyte (HCM) by the formation of glycogen filled vacuoles in myocytes. Most of the mutations on PRKAG2 are gain of function (GOF) mutations. Numerous human PRKAG2 GOF mutations have been identified and each of these mutations is associated with a point mutation within the PRKAG2 gene resulting in an amino acid substitution of the PRKAG2 protein. Most commonly identified amino acid substitutions of the PRKAG2 protein include H142R, R302Q, L341S, H383R, R384T, T400N, H401D, K475E, K485I, Y487H, N488I, S548P, and R531G. These PRKAG2 GOF mutations in cardiac cells result in glycogen accumulation in the heart muscle cells, leading to glycogen storage cardiomyopathy.

Current treatments for PRKAG2 cardiac syndrome that include standard heart failure and antiarrhythmic treatment, pacemaker, defibrillator implantation and surgical ablation alleviate the symptoms but do not treat the genetic cause of the cardiac abnormalities. However, there are no specific treatments available that target PRKAG2 mRNA. There is a need to develop therapeutics for treating cardiomyopathy caused by PRKAG2 cardiac syndrome.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

In the present disclosure, methods and compositions of antibody-oligonucleotide conjugates (AOC) targeting PRKAG2 mRNA are provided to inhibit the expression of PRKAG2. In addition, the present disclosure provides methods and compositions to treat cardiomyopathy caused by mutations of PRKAG2 with antibody oligonucleotide conjugates to deliver nucleic acids that target the expression of PRKAG2 in tissue. In addition, the present disclosure provides methods and compositions to treat cardiomyopathy caused by PRKAG2 cardiac syndrome with antibody-oligonucleotide conjugates to deliver nucleic acids that target the expression of PRKAG2 in cardiac tissue.

Disclosed herein, in some aspects, includes methods of treating cardiomyopathy in a subject in need thereof by administering the subject an effective amount of a polynucleotide conjugate, which comprises an anti-transferrin receptor antibody or antigen-binding fragment thereof conjugated to a polynucleotide molecule. In some instances, the polynucleotide molecule hybridizes to a target sequence of PRKAG2 mRNA and mediates RNA interference against the PRKAG2 mRNA in a muscle cell, thereby treating cardiomyopathy in the subject. In some instances, the cardiomyopathy is caused by a glycogen storage disease. In some instances, the cardiomyopathy is caused by PRKAG2 syndrome or PRKAG2 cardiac syndrome. In some instances, PRKAG2 syndrome or PRKAG2 cardiac syndrome is caused by a mutated PRKAG2 that has a gain of function. In some instances, the mutated PRKAG2 comprises R531G mutation. In some instances, the polynucleotide conjugate is administered parenterally, orally, intranasally, buccally, rectally, transdermally, intravenously, subcutaneously, or intrathecally. In some instances, the polynucleotide molecule is a double-stranded inhibitory RNA molecule (e.g., siRNA) comprising a guide strand and a passenger strand, wherein the guide strand comprises a nucleic acid sequence having at least 85%, 90%, or 95% sequence identity to a nucleic acid sequence selected from SEQ ID NOs: 58, 61, 62, 64, 77, and 98. In some instances, the guide strand comprises a nucleic acid sequence having at least 85%, 90%, or 95% sequence identity to a nucleic acid sequence selected from SEQ ID NOs: 209-220. In some instances, the passenger strand comprises a nucleic acid sequence having at least 85%, 90%, or 95% sequence identity to a nucleic acid sequence selected from SEQ ID NOs: 221-232. In some instances, the polynucleotide conjugate reduces the mRNA expression level of the PRKAG2 mRNA in the muscle cell at least 40%, 50%, or 60% compared to PRKAG2 mRNA level before administration. In some instances, the effective amount of the polynucleotide conjugate is effective to reduce the glycogen accumulation in a skeletal muscle tissue or a cardiac tissue of the subject. In some instances, the subject is a human. In some instances, the polynucleotide conjugate is administered with a dose of about 1, 2, 3, or 4 mg/kg. In some instances, the polynucleotide conjugate is administered with a dose of about 1-3 mg siRNA/kg subcutaneously. In some instances, the polynucleotide conjugate is administered with a dose of about 1 mg siRNA/kg intravenously. In some instances, the polynucleotide conjugate is administered with a dose of about 1 mg siRNA/kg subcutaneously. In some instances, the polynucleotide conjugate is administered once in 1, 3, 5, 10, 12, 24, or 36 weeks. In some instances, the polynucleotide conjugate is administered once in about 4, 8, 12, 16, 20, or 24 weeks.

Also disclosed herein, in some aspects, includes a method of inhibiting or reducing glycogen accumulation in a skeletal muscle tissue or a cardiac tissue of a subject in need thereof by administering to the subject an effective amount of a polynucleotide conjugate comprising an anti-transferrin receptor antibody or antigen-binding fragment thereof conjugated to a polynucleotide molecule. In some instances, the polynucleotide molecule hybridizes to a target sequence of PRKAG2 mRNA and mediates RNA interference against the PRKAG2 mRNA in a muscle cell, thereby inhibiting or reducing glycogen accumulation in the skeletal muscle tissue or the cardiac tissue of the subject. In some instances, the subject is affected by the PRKAG2 syndrome or PRKAG2 cardiac syndrome. In some instances, the subject comprises R531G mutation in a PRKAG2 gene. In some instances, the polynucleotide molecule is a double-stranded inhibitory RNA molecule (e.g., siRNA) comprising a guide strand and a passenger strand, wherein the guide strand comprises a nucleic acid sequence at least 85%, 90%, or 95% complementary to a nucleic acid sequence selected from SEQ ID NOs: 58, 61, 62, 64, 77, and 98. In some instances, the guide strand comprises a nucleic acid sequence at least 85%, 90%, or 95% complementary to a nucleic acid sequence selected from SEQ ID NOs: 209-220. In some instances, the passenger strand comprises a nucleic acid sequence at least 85%, 90%, or 95% complementary to a nucleic acid sequence selected from SEQ ID NOs: 221-232. In some instances, the polynucleotide conjugate reduces the mRNA expression level of the PRKAG2 mRNA in the skeletal muscle tissue or the cardiac tissue at least 40%, 50%, or 60% compared to PRKAG2 mRNA level before administration. In some instances, the administration is via an intravenous injection subcutaneous injection. In some instances, the administration is via a single subcutaneous injection of a desired dose, or multiple subcutaneous injections with a total sum of injected amount of the desired dose. In some instances, the polynucleotide conjugate is administered with a dose of about 1, 2, 3, or 4 mg/kg. In some instances, the polynucleotide conjugate is administered with a dose of about 1-3 mg siRNA/kg subcutaneously. In some instances, the polynucleotide conjugate is administered with a dose of about 1 mg siRNA/kg intravenously. In some instances, the polynucleotide conjugate is administered with a dose of about 1 mg siRNA/kg subcutaneously. In some instances, the polynucleotide conjugate is administered once in 1, 3, 5, 10, 12, 24, or 36 weeks. In some instances, the polynucleotide conjugate is administered once in about 4, 8, 12, 16, 20, or 24 weeks.

Also disclosed herein, in some aspects, includes a use of a polynucleotide conjugate in the manufacture of a medicament for treating cardiomyopathy in a subject in need thereof. In some instances, the polynucleotide conjugate comprises an anti-transferrin receptor antibody or antigen-binding fragment thereof conjugated to a polynucleotide molecule. In some instances, the polynucleotide molecule hybridizes to a target sequence of PRKAG2 mRNA and mediates RNA interference against the PRKAG2 mRNA in a muscle cell. In some instances, the cardiomyopathy is caused by a glycogen storage disease. In some instances, the cardiomyopathy is caused by PRKAG2 syndrome or PRKAG2 cardiac syndrome. In some instances, the PRKAG2 syndrome or PRKAG2 cardiac syndrome is caused by a mutated PRAKG2 that has a gain of function. In some instances, the mutated PRKAG2 comprises R531G mutation. In some instances, the polynucleotide conjugate is formulated for administration parentally, orally, intranasally, buccally, rectally, transdermally, intravenously, subcutaneously, or intrathecally. In some instances, the polynucleotide molecule is a double-stranded inhibitory RNA molecule comprising a guide strand and a passenger strand, wherein the guide strand comprises a nucleic acid sequence having at least 85%, 90%, or 95% sequence identity to a nucleic acid sequence selected from SEQ ID NOs: 58, 61, 62, 64, 77, and 98. In some instances, the guide strand comprises a nucleic acid sequence having at least 85%, 90%, or 95% sequence identity to a nucleic acid sequence selected from SEQ ID NOs: 209-220. In some instances, the passenger strand comprises a nucleic acid sequence having at least 85%, 90%, or 95% sequence identity to a nucleic acid sequence selected from SEQ ID NOs: 221-232. In some instances, the polynucleotide conjugate reduces the mRNA expression level of the PRKAG2 mRNA in the muscle cell at least 40%, 50%, or 60% compared to PRKAG2 mRNA level before administration. In some instances, the effective amount of the polynucleotide conjugate is effective to reduce the glycogen accumulation in a skeletal muscle tissue or a cardiac tissue of the subject. In some instances, the polynucleotide conjugate is administered with a dose of about 1, 2, 3, or 4 mg/kg. In some instances, the muscle cell is an extrahepatic tissue, a skeletal muscle tissue, or cardiac tissue of the subject. In some instances, the subject is a human. In some instances, the administration is via an intravenous injection or subcutaneous injection. In some instances, the polynucleotide conjugate is administered with a dose of about 1-3 mg siRNA/kg intravenously. In some instances, the polynucleotide conjugate is administered with a dose of about 1-3 mg siRNA/kg subcutaneously. In some instances, the polynucleotide conjugate is administered with a dose of about 1 mg siRNA/kg intravenously. In some instances, the polynucleotide conjugate is administered with a dose of about 1 mg siRNA/kg subcutaneously. In some instances, the polynucleotide conjugate is administered once in 1, 3, 5, 10, 12, 24, or 36 weeks. In some instances, the polynucleotide conjugate is administered once in about 4, 8, 12, 16, 20, or 24 weeks.

Also disclosed herein, in some aspects, includes a use of a polynucleotide conjugate in the manufacture of a medicament for inhibiting or reducing glycogen accumulation in a skeletal muscle tissue or a cardiac tissue of a subject in need thereof. In some instances, the polynucleotide conjugate comprises an anti-transferrin receptor or antigen-binding fragment thereof conjugated to a polynucleotide molecule. In some instances, the polynucleotide molecule hybridizes to a target sequence of PRKAG2 mRNA and mediates RNA interference against the PRKAG2 mRNA. In some instances, the subject is affected by PRKAG2 syndrome or PRKAG2 cardiac syndrome. In some instances, the PRKAG2 syndrome or PRKAG2 cardiac syndrome is caused by a mutated PRKAG2 that has a gain of function. In some instances, the mutated PRKAG2 comprises R531G mutation. In some instances, the polynucleotide molecule is a double-stranded inhibitory RNA molecule comprising a guide strand and a passenger strand, wherein the guide strand comprises a nucleic acid sequence having at least 85%, 90%, or 95% sequence identity to a nucleic acid sequence selected from SEQ ID NOs: 58, 61, 62, 64, 77, and 98. In some instances, the guide strand comprises a nucleic acid sequence having at least 85%, 90%, or 95% sequence identity to a nucleic acid sequence selected from SEQ ID NOs: 209-220. In some instances, the passenger strand comprises a nucleic acid sequence having at least 85%, 90%, or 95% sequence identity to a nucleic acid sequence selected from SEQ ID NOs: 221-232. In some instances, the polynucleotide conjugate reduces the mRNA expression level of the PRKAG2 mRNA in the skeletal muscle tissue or the cardiac tissue at least 40%, 50%, or 60% compared to PRKAG2 mRNA level before administration. In some instances, the polynucleotide conjugate is formulated to be administered parenterally, orally, intranasally, buccally, rectally, transdermally, intravenously, subcutaneously, or intrathecally. In some instances, the administration is via an intravenous injection or subcutaneous injection. In some instances, the polynucleotide conjugate is administered with a dose of about 1, 2, 3, or 4 mg/kg. In some instances, the subject is a human. In some instances, the polynucleotide conjugate is administered with a dose of about 1-3 mg siRNA/kg intravenously. In some instances, the polynucleotide conjugate is administered with a dose of about 1-3 mg siRNA/kg subcutaneously. In some instances, the polynucleotide conjugate is administered with a dose of about 1 mg siRNA/kg intravenously. In some instances, the polynucleotide conjugate is administered with a dose of about 1 mg siRNA/kg subcutaneously. In some instances, the polynucleotide conjugate is administered once in 1, 3, 5, 10, 12, 24, or 36 weeks. In some instances, the polynucleotide conjugate is administered once in about 4, 8, 12, 16, 20, or 24 weeks.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIGS. 1A-1B show dose response and PD/PK activity of anti-transferrin receptor antibody conjugated with siRNA targeting PRKAG2 (siPRKAG2-AOC) in WT mice. FIG. 1A is a plot showing siRNA concentration in the heart muscle (round dot) and gastrocnemius muscle (triangle) at 28 days after a dose of 0.03, 0.1, 0.3, 1, or 2 mg/kg (siRNA dose). FIG. 1B is a plot showing PRKAG2 mRNA expression levels in the heart muscle (round dot) and gastrocnemius muscle (triangle) at 28 days after a dose of 0.03, 0.1, 0.3, 1, or 2 mg/kg (siRNA dose).

FIGS. 2A-2B show glucose content in the quadriceps muscle tissue and the heart tissue obtained from WT mice and TG mice (PRKAG2 mutant mice carrying a R528G point mutation). 8-12 weeks-old mice were injected with PBS or siPRKAG2-AOC (at 3 mg/kg) for 24 weeks (two doses 12 weeks apart), n=16-20/group. * indicates p<0.05. FIG. 2A is a bar graph showing glucose content in the quadriceps muscle tissue obtained from WT mice and TG mice. FIG. 2B is a bar graph showing glucose content in the heart tissue obtained from WT mice and TG mice.

FIGS. 3A-3D show cardiac function in WT mice and PRKAG2 mutant (TG) mice. 8-12 weeks-old mice were injected with PBS or siPRKAG2-AOC (at 3 mg/kg) for 32 weeks (3 doses; 12 weeks apart). For diastolic function n=5-7 for WT and for n=9 for TG mice were injected. For P wave n=16-20/group were sampled. * indicates p<0.05. FIG. 3A is a bar graph showing ejection fraction of WT mice and TG mice after injection with of PBS (vehicle) or siPRKAG2-AOC. FIG. 3B shows representative images of cardiac echo-doppler for WT mice and TG mice after injection with PBS (vehicle) or siPRKAG2-AOC. FIG. 3C is a bar graph showing E/A ratio obtained from WT mice and TG mice after injection with PBS (vehicle) or siPRKAG2-AOC. FIG. 3D shows a bar graph showing P wave amplitude (mV) in WT mice and TG mice after injection with PBS (vehicle) or siPRKAG2-AOC.

FIGS. 4A-4E show results of anti-human transferrin receptor antibody conjugated with siRNA targeting PRKAG2 (siPRKAG2-hAOC) treatment in non-human primate (NHP, cynomolgus monkeys). FIG. 4A is a bar graph showing PRKAG2 mRNA expression levels in heart following a single dose of siPRKAG2-hAOC in male cynomolgus monkeys relative to control. FIG. 4B is a JESS capillary western blot image of cardiac PRKAG2 protein expression in cynomolgus monkeys. FIG. 4C is a time course graph showing the animal body weights during the study period. FIG. 4D is a bar graph showing PR intervals measured by ECG. FIG. 4E is a bar graph showing QTc Intervals measured by ECG. Bars represent mean±SD and dots represent individual animals.

FIGS. 5A-5C show bar graphs of PRKAG2 mRNA expression levels in heart apex (FIG. 5A), left ventricular (LV) Free Wall (FIG. 5B) and right ventricle (RV) (FIG. 5C) following intravenous or subcutaneous administration of a single dose of siPRKAG2-hAOC in female cynomolgus monkey relative to sterile saline control.

FIGS. 6A-6B show line graphs of PRKAG2 mRNA expression levels in gastrocnemius muscle (a), and vastus lateralis (b) following intravenous or subcutaneous administration of a single dose of siPRKAG2-hAOC in female cynomolgus monkey relative to predose expression. Bars represent mean±SD and symbols represent individual animals.

FIGS. 7A-7B show bar graphs of PRKAG2 mRNA expression levels in latissimus dorsi (FIG. 7A) and biceps (FIG. 7B) following intravenous or subcutaneous administration of a single dose of siPRKAG2-hAOC in female cynomolgus monkey relative to vehicle. Bars represent mean±SD and symbols represent individual animals.

FIGS. 8A-8B show bar graphs of PRKAG2 mRNA expression levels in liver (FIG. 8A) and kidney (FIG. 8B) following intravenous or subcutaneous administration of a single dose of siPRKAG2-hAOC in female cynomolgus monkey relative to vehicle. Bars represent mean±SD and symbols represent individual animals.

FIG. 9 shows a line graph of PRKAG2 siRNA concentrations in plasma following intravenous or subcutaneous administration of a single dose of siPRKAG2-hAOC in female cynomolgus monkeys. Bars represent mean±SD.

FIG. 10 shows a bar graph of PRKAG2 siRNA concentrations in tissues at day 28 following intravenous or subcutaneous administration of a single dose of siPRKAG2-hAOC in female cynomolgus monkeys. Bars represent mean±SD.

FIG. 11 shows a line graph of PRKAG2 siRNA concentrations in gastroc tissue at day 7, 21 and 28 following intravenous or subcutaneous administration of a single dose administration of siPRKAG2-hAOC in female cynomolgus monkeys. Bars represent mean SD.

FIG. 12 shows a line graph of PRKAG2 siRNA concentrations in vastus lateralis tissue at day 7, 21 and 28 following intravenous or subcutaneous administration of a single dose of siPRKAG2-hAOC in female cynomolgus monkeys. Bars represent mean±SD.

FIG. 13 shows a line graph of PRKAG2 siRNA concentrations in plasma following a single dose of either intravenous or subcutaneous administration of mTfR1-mAb-siPRKAG2 DAR1 to mice. Bars represent mean±SD.

FIG. 14 shows a bar graph of PRKAG2 siRNA concentrations in tissue at 15 days following intravenous or subcutaneous administration of a single dose of 2 mg/kg mAb-siPRKAG2 DAR1 or three doses of 0.67 mg/kg of subcutaneous administration of mAb-siPRKAG2 DAR1 to mice.

FIG. 15 shows a bar graph of PRKAG2 mRNA expression levels in gastroc or quadricep muscles at 15 days following a single dose of either intravenous or subcutaneous administration of 2 mg/kg mAb-siPRKAG2 DAR1 or three doses of 0.67 mg/kg of subcutaneous administration of mAb-siPRKAG2 DAR1 to mice.

### DETAILED DESCRIPTION OF THE DISCLOSURE

Mutations in PRKAG2, the gene encoding the γ2 regulatory subunit of AMPK, cause cardiac hypertrophy and electrophysiologic abnormalities, particularly preexcitation (Wolff-Parkinson-White syndrome) and atrioventricular conduction block. Because of the complex electrophysiological impact of the disease, an incidence rate of premature (<40 years) sudden cardiac death (SCD) as high as 20% has been suggested. Most of these carriers end up needing a pacemaker, ablation surgery and/or implantation of a defibrillator. Incidents of heart failure, and atrial fibrillation are also very high.

The PRKAG2 isoform is predominantly expressed in the heart. Transgenic mice overexpressing the human PRKAG2 disease variants, recapitulate the human syndrome and display excessive accumulation of glycogen in the heart, hypertrophy, and preexcitation. The activity of AMPK is increased in the hearts expressing the disease variants, suggesting a gain of function role. Furthermore, several pan-AMPK activators have been previously developed to treat diabetes, but they all resulted in cardiac hypertrophy (rodents, non-human primates (NHP) and humans) and were not further pursued. Mice with cardiac PRKAG2 deletion have no adverse phenotype and normal cardiac function. The lack of PRKAG2 is compensated for by an increase in PRKAG1 (skeletal muscle isoform), thus the total AMPK activity remains unchanged.

Clinical presentation ranges from asymptomatic condition to sudden cardiac death (SCD). PRKAG2 syndrome onset of symptoms frequently occurs within the first 3 decades of age, and it is often characterized by tachyarrhythmias and bradyarrhythmia. Much less frequently, heart failure symptoms or SCD can be the first manifestations of the disease. Prolonged dynamic ECG monitoring and exercise stress testing could be useful tools in those patients with syncope, palpitations, or with a familial history of SCD. Ultrasound imaging and cardiovascular magnetic resonance can be considered gold-standard diagnostic techniques for the identification and characterization of cardiac hypertrophy.

Nucleic acid based therapy (e.g., RNAi) is a targeted therapy with high selectivity and specificity. However, in some instances, nucleic acid therapy is also hindered by poor intracellular uptake, limited blood stability and non-specific immune stimulation. To address these issues, various modifications of the nucleic acid composition are explored, such as, for example, novel linkers for better stabilizing and/or lower toxicity, optimization of binding moiety for increased target specificity and/or target delivery, and/or nucleic acid polymer modifications for increased stability and/or reduced off-target effect.

In some aspects, the arrangement or order of the different components that make up the nucleic acid composition further affects intracellular uptake, stability, toxicity, efficacy, and/or non-specific immune stimulation. For example, if the nucleic acid component includes a binding moiety, a polymer, and a polynucleic acid molecule (or polynucleotide), the order or arrangement of the binding moiety, the polymer, and/or the polynucleic acid molecule (or polynucleotide) (e.g., binding moiety-polynucleic acid molecule-polymer, binding moiety-polymer-polynucleic acid molecule, or polymer-binding moiety-polynucleic acid molecule) further effects intracellular uptake, stability, toxicity, efficacy, and/or non-specific immune stimulation.

The present disclosure provides, in certain aspects, oligonucleotide molecules or antibody-oligonucleotides conjugates (AOC) targeting PRKAG2 mRNA, which are capable of inhibiting or modulating the expression of PRKAG2. In some aspects, the present disclosure provides methods of modulating PRKAG2 mRNA expression using the oligonucleotide molecules or antibody-oligonucleotides conjugates (AOC) targeting PRKAG2 mRNA. In some aspects, described herein include polynucleic acid molecules (interchangeably used with the terms “polynucleotide” or “oligonucleotide”) and polynucleic acid molecule conjugates for the treatment of cardiomyopathy. In some instances, the polynucleic acid molecule conjugates described herein have or show enhanced intracellular uptake, stability, and/or efficacy. In some cases, the polynucleic acid molecule conjugates comprise an antibody or antigen binding fragment thereof conjugated to a polynucleic acid molecule. In some cases, the polynucleic acid molecules hybridize to target sequences of PRKAG2 mRNA, preferably human PRKAG2 mRNA. In some cases, the polynucleic acid molecules that hybridize to target sequences of mutated PRKAG2 that has a gain-of-function or a target sequence of the PRKAG2 mRNA comprising a gain-of-function mutation.

In some aspects, the present disclosure further provides treatment of cardiomyopathy or its symptoms thereof, associated with PRKAG2 expression in the heart muscle cells. In certain instances, the cardiomyopathy is caused by PRKAG2 syndrome or PRKAG2 cardiac syndrome. In some aspects, the present disclosure further provides treatment of cardiomyopathy or its symptoms thereof caused by a glycogen storage disease, by administering to a subject a polynucleic acid molecule or a polynucleic acid molecule conjugate described herein. In some aspects, the present disclosure further provides treatment of cardiomyopathy caused by PRKAG2 syndrome or PRKAG2 cardiac syndrome by administering to a subject a polynucleic acid molecule or a polynucleic acid molecule conjugate. In some aspects, the present disclosure further provides treatment of cardiomyopathy caused by a PRKAG gain-of-function mutation by administering to a subject a polynucleic acid molecule or a polynucleic acid molecule conjugate.

In some aspects, the present disclosure further provides methods of reducing glycogen accumulation in a skeletal muscle tissue or a cardiac tissue of a subject by administering to a subject a polynucleic acid molecule or a polynucleic acid molecule conjugate described herein.

### Polynucleic Acid Molecules

In certain aspects, a polynucleic acid molecule hybridizes to a target sequence of PRKAG2 gene (e.g., PRKAG2 mRNA). In some instances, a polynucleic acid molecule described herein hybridizes to a target sequence of human PRKAG2 gene (e.g., human PRKAG2 mRNA) and reduces PRKAG2 mRNA expression in cardiac muscle cells.

In certain aspects, a polynucleic acid molecule hybridizes to a target sequence of PRKAG2 mRNA. In some instances, a polynucleic acid molecule described herein hybridizes to a target sequence of a PRKAG2 variant comprising a point mutation in the PRKAG2 gene and reduces the expression of PRKAG2 mRNA in cardiac muscle cells.

In some aspects, the polynucleic acid molecule comprises a nucleic acid sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1-102. In some instances, the polynucleic acid molecule comprises a nucleic acid sequence comprising at least 14, 15, 16, 17, 18, 19 consecutive nucleotide sequences with no more than 1, 2, 3 mismatches from SEQ ID NOs: 1-102. In some aspects, the polynucleic acid molecule comprises a nucleic acid sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 103-204. In some instances, the polynucleic acid molecule comprises a nucleic acid sequence comprising at least 14, 15, 16, 17, 18, 19 consecutive nucleotide sequences with no more than 1, 2, or 3 mismatches from SEQ ID NOs: 103-204.

In some aspects, the polynucleic acid molecule is a single-stranded antisense oligonucleotide (ASO) hybridizing to a target sequence of PRKAG2 mRNA. In some instances, the ASO comprises a nucleic acid sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1-102. In some instances, the ASO comprises a nucleic acid sequence comprising at least 14, 15, 16, 17, 18, or 19 consecutive nucleotide sequences with no more than 1, 2, 3 mismatches from SEQ ID NOs: 1-102.

In some instances, the ASO is a gapmer or a mixmer. In some instances, the ASO comprises a central region of consecutive DNA nucleotides flanked by a 5′-wing region and 3′-wing region, and the flanked 5′ and/or 3′ wing region comprises one or more modified nucleotides (e.g., locked nucleic acid (LNA) or 2′-methoxyethyl (2′-MOE) RNA). In some instances, the locked nucleic acid comprises at least one or more of a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA. In some instances, the ASO comprises 3-10-3 configurations (3 nucleotides for 5′-flanked region, 10 nucleotides of central region, and 3 nucleotides for 3′-flanked region), 5-10-5 configuration (5 nucleotides for 5′-flanked region, 10 nucleotides of central region, and 5 nucleotides for 3′-flanked region), or X-Y-Z configuration where X can be 1-10 nucleotides, Y can be 8-20 nucleotides, Z can be 1-10 nucleotides. In some instances, all nucleotides in the wing region are modified nucleotides (e.g., 2′-MOE modified RNA).

In some aspects, the polynucleic acid molecule is a double-stranded polynucleotide, comprising a first polynucleotide and a second polynucleotide. In some instances, the first polynucleotide comprises a nucleic acid sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1-102. In some instances, the first nucleotide comprises a nucleic acid sequence comprising at least 14, 15, 16, 17, 18, or 19 consecutive nucleotide sequences with no more than 1, 2, 3 mismatches from SEQ ID NOs: 1-102. In some cases, the second polynucleotide comprises a nucleic acid sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 103-204. In some instances, the second nucleotide comprises a nucleic acid sequence comprising at least 14, 15, 16, 17, 18, 19 consecutive nucleotide sequences with no more than 1, 2, 3 mismatches from SEQ ID NOs: 103-204.

In some aspects, the polynucleic acid molecule described herein comprises RNA or DNA. In some cases, the polynucleic acid molecule comprises RNA. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the polynucleic acid molecule is a phosphorodiamidate morpholino oligomer (PMO), which comprise short single-stranded oligonucleotide analogs that are built upon a backbone of morpholine rings connected by phosphorodiamidate linkages. In some instances, the RNA comprises siRNA. In some instances, the polynucleic acid molecule comprises siRNA.

In some aspects, the polynucleic acid molecule comprises a sense strand (e.g., a passenger strand) and an antisense strand (e.g., a guide strand). In some instances, the sense strand (e.g., the passenger strand) comprises a nucleic acid sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 103-204. In some instances, the sense strand (e.g., the passenger strand) comprises a nucleic acid sequence comprising at least 14, 15, 16, 17, 18, or 19 consecutive nucleotide sequences with no more than 1, 2, 3 mismatches from SEQ ID NOs: 103-204. In some instances, the antisense strand (e.g., the guide strand) comprises a nucleic acid sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1-102. In some instances, the antisense strand (e.g., the guide strand) comprises a nucleic acid sequence comprising at least 14, 15, 16, 17, 18, 19 consecutive nucleotide sequences with no more than 1, 2, 3 mismatches from SEQ ID NOs: 1-102.

In some instances, the siRNA comprises sense (passenger) strand and antisense (guide) strand as presented in Table 10.

In some aspects, the polynucleic acid molecule is from about 8 to about 50 nucleotides in length. In some aspects, the polynucleic acid molecule is from about 10 to about 50 nucleotides in length. In some instances, the polynucleic acid molecule is from about 10 to about 30, from about 15 to about 30, from about 18 to about 25, form about 18 to about 24, from about 19 to about 23, or from about 20 to about 22 nucleotides in length.

In some aspects, the polynucleic acid molecule is about 50 nucleotides in length. In some instances, the polynucleic acid molecule is about 45 nucleotides in length. In some instances, the polynucleic acid molecule is about 40 nucleotides in length. In some instances, the polynucleic acid molecule is about 35 nucleotides in length. In some instances, the polynucleic acid molecule is about 30 nucleotides in length. In some instances, the polynucleic acid molecule is about 25 nucleotides in length. In some instances, the polynucleic acid molecule is about 20 nucleotides in length. In some instances, the polynucleic acid molecule is about 19 nucleotides in length. In some instances, the polynucleic acid molecule is about 18 nucleotides in length. In some instances, the polynucleic acid molecule is about 17 nucleotides in length. In some instances, the polynucleic acid molecule is about 16 nucleotides in length. In some instances, the polynucleic acid molecule is about 15 nucleotides in length. In some instances, the polynucleic acid molecule is about 14 nucleotides in length. In some instances, the polynucleic acid molecule is about 13 nucleotides in length. In some instances, the polynucleic acid molecule is about 12 nucleotides in length. In some instances, the polynucleic acid molecule is about 11 nucleotides in length. In some instances, the polynucleic acid molecule is about 10 nucleotides in length. In some instances, the polynucleic acid molecule is about 8 nucleotides in length. In some instances, the polynucleic acid molecule is between about 8 and about 50 nucleotides in length. In some instances, the polynucleic acid molecule is between about 10 and about 50 nucleotides in length. In some instances, the polynucleic acid molecule is between about 10 and about 45 nucleotides in length. In some instances, the polynucleic acid molecule is between about 10 and about 40 nucleotides in length. In some instances, the polynucleic acid molecule is between about 10 and about 35 nucleotides in length. In some instances, the polynucleic acid molecule is between about 10 and about 30 nucleotides in length. In some instances, the polynucleic acid molecule is between about 20 and about 30 nucleotides in length. In some instances, the polynucleic acid molecule is between about 10 and about 25 nucleotides in length. In some instances, the polynucleic acid molecule is between about 10 and about 20 nucleotides in length. In some instances, the polynucleic acid molecule is between about 15 and about 25 nucleotides in length. In some instances, the polynucleic acid molecule is between about 15 and about 30 nucleotides in length. In some instances, the polynucleic acid molecule is between about 12 and about 30 nucleotides in length.

In some aspects, the polynucleic acid molecule comprises a first polynucleotide and a second polynucleotide. In some instances, the first polynucleotide is a sense strand (passenger strand) and the second polynucleotide is an antisense strand (guide strand) of a double stranded inhibitory RNA (dsRNA) or an siRNA.

In some aspects, each of the first and/or second polynucleotide is from about 8 to about 50 nucleotides in length. In some aspects, each of the first and/or second polynucleotide is from about 10 to about 50 nucleotides in length. In some instances, each of the first and/or second polynucleotide is from about 10 to about 30, from about 15 to about 30, from about 18 to about 25, form about 18 to about 24, from about 19 to about 23, or from about 20 to about 22 nucleotides in length. In some instances, each of the first and/or second polynucleotide is about 50, 45, 40, 35, 30, 25, 24, 23, 22, 21, 20, 19, 18, 17 nucleotides in length.

In some aspects, the polynucleic acid molecule comprises a first polynucleotide and a second polynucleotide. In some instances, the polynucleic acid molecule further comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides. In some aspects, the polynucleic acid molecule comprises a sense strand and an antisense strand, and the antisense strand includes two non-base pairing nucleotides as an overhang at the 3′-end while the sense strand has no overhang. Optionally, in such embodiments, the non-base pairing nucleotides have a sequence of TT, dTdT, or UU. In some aspects, the polynucleic acid molecule comprises a sense strand and an antisense strand, and the sense strand has one or more nucleotides at the 5′-end that are complementary to the antisense sequence.

In some aspects, the nucleic acid sequence of the polynucleic acid molecule is at least 40%, 50%, 55%, 60%, 65%, 70%, 75%, 80%, 85%, 90%, 95%, 98%, 99%, or 99.5% complementary to a target sequence of PRKAG2 mRNA. In some aspects, the target sequence of PRKAG2 mRNA is a nucleic acid sequence of about 10-50 nucleotides in length, about 15-50 nucleotides in length, 15-40 nucleotides in length, 15-30 nucleotides in length, or 15-25 nucleotides in length sequences in PRKAG2 mRNA, in which the first nucleotide of the target sequence starts at any nucleotide in PRKAG2 mRNA transcript in the coding region, or in the 5′ or 3′-untraslated region (UTR).

In some aspects, the nucleic acid sequence of the polynucleic acid molecule is at least 50% complementary to a target sequence described herein. In some aspects, the nucleic acid sequence of the polynucleic acid molecule is at least 60% complementary to a target sequence described herein. In some aspects, the nucleic acid sequence of the polynucleic acid molecule is at least 70% complementary to a target sequence described herein. In some aspects, the nucleic acid sequence of the polynucleic acid molecule is at least 80% complementary to a target sequence described herein. In some aspects, the nucleic acid sequence of the polynucleic acid molecule is at least 90% complementary to a target sequence described herein. In some aspects, the nucleic acid sequence of the polynucleic acid molecule is at least 95% complementary to a target sequence described herein. In some aspects, the nucleic acid sequence of the polynucleic acid molecule is at least 99% complementary to a target sequence described herein. In some instances, the nucleic acid sequence of the polynucleic acid molecule is 100% complementary to a target sequence described herein.

In some aspects, the nucleic acid sequence of the polynucleic acid molecule has 5 or less mismatches to a target PRKAG2 mRNA sequence described herein. In some aspects, the nucleic acid sequence of the polynucleic acid molecule has 4 or less mismatches to a target PRKAG2 mRNA sequence described herein. In some instances, the nucleic acid sequence of the polynucleic acid molecule has 3 or less mismatches to a target PRKAG2 mRNA sequence described herein. In some cases, the nucleic acid sequence of the polynucleic acid molecule has 2 or less mismatches to a target PRKAG2 mRNA sequence described herein. In some cases, the nucleic acid sequence of the polynucleic acid molecule has 1 or less mismatches to a target PRKAG2 mRNA sequence described herein.

In some aspects, a group of polynucleic acid molecules among all the polynucleic acid molecules that potentially binds to the target sequence of PRKAG2 mRNA are selected to generate a polynucleic acid molecule library. In certain embodiments, such selection process is conducted in silico via one or more steps of eliminating less desirable polynucleic acid molecules from candidates using one or more selection criteria, e.g., similarity to miRNA sequences, expected off-target effects, etc.

In some aspects, the specificity of the polynucleic acid molecule that hybridizes to a target sequence described herein is a 95%, 98%, 99%, 99.5% or 100% sequence complementarity of the polynucleic acid molecule to a target PRKAG2 mRNA sequence. In some instances, the hybridization is a high stringent hybridization condition.

In some aspects, the polynucleic acid molecule has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence, a cellular protein, or other moiety. In some instances, an “off-target effect” occurs when there is a simultaneous degradation of other transcripts due to partial homology or complementarity between that other transcript and the sense and/or antisense strand of the polynucleic acid molecule.

In some aspects, the polynucleic acid molecule comprises natural or synthetic or artificial nucleotide analogues or bases. In some cases, the polynucleic acid molecule comprises combinations of DNA, RNA and/or synthetic or artificial nucleotide analogues or bases. In some instances, the synthetic or artificial nucleotide analogues or bases comprise modifications at one or more of a ribose moiety, a phosphate moiety, a base moiety, or a combination thereof.

In some aspects, nucleotide analogues or artificial nucleotide base comprise a nucleotide with a modification at a 2′ hydroxyl group of the ribose moiety. In some instances, the modification includes an H, OR, R, halo, SH, SR, NH₂, NHR, NR₂, or CN, wherein R is an alkyl moiety. Exemplary alkyl moieties include, but are not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, and disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety. Exemplary chemical structures of a 2′-O-methyl modification of an adenosine molecule and 2′O-methoxyethyl modification of a uridine are illustrated below.

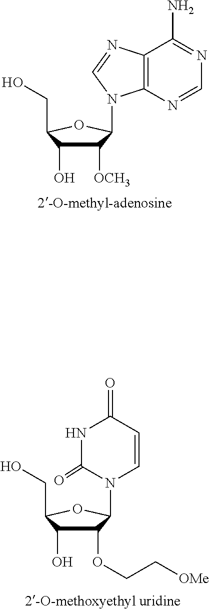

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

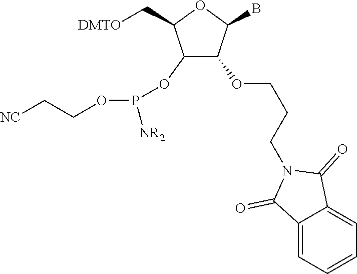

### 2′-O-aminopropyl nucleoside phosphoramidite

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

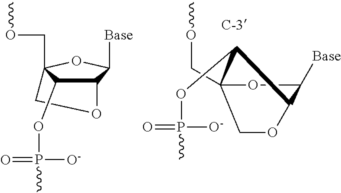

### LNA (Locked Nucleic Acids)

In some instances, the modification at the 2′ hydroxyl group comprises ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA. Exemplary chemical structures of the ENA and bridged nucleic acids are illustrated below.

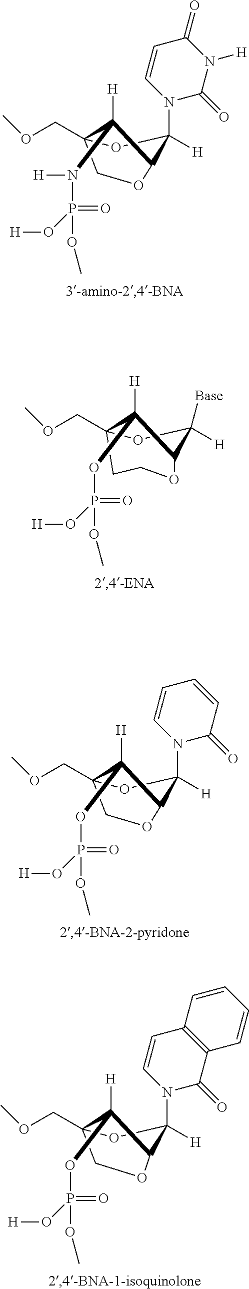

In some aspects, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O— dimethylaminoethyloxyethyl (2′-O-DMAEOE), or 2′-O-N-methylacetamido (2′-O-NMA).

In some aspects, nucleotide analogues comprise modified bases such as, but not limited to, 5-propynyluridine, 5-propynylcytidine, 6-methyladenine, 6-methylguanine, N, N, -dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what is known in the art as universal bases. By way of example, universal bases include but are not limited to 3-nitropyrrole, 5-nitroindole, or nebularine.

In some aspects, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholinos or phosphorodiamidate morpholino oligomers (PMOs) comprise synthetic molecules whose structure mimics a natural nucleic acid structure by deviating from the normal sugar and phosphate structures. In some instances, the five-member ribose ring is substituted with a six-member morpholino ring containing four carbons, one nitrogen and one oxygen. In some cases, the ribose monomers are linked by a phosphorodiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

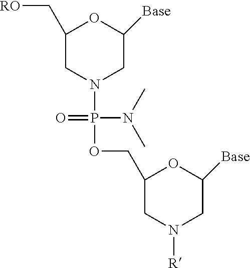

### Morpholino

In some aspects, a peptide nucleic acid (PNA) does not contain a sugar ring or a phosphate linkage and the bases are attached and appropriately spaced by oligoglycine-like molecules, therefore, eliminating a backbone charge.

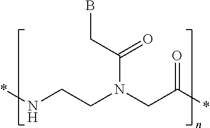

In some aspects, one or more modifications optionally occur at the internucleotide linkage. In some instances, modified internucleotide linkage types include, but are not limited to, phosphorothioates, phosphorodithioates, methylphosphonates, 5′-alkylenephosphonates, 5′-methylphosphonate, 3-alkylene phosphonates, borontrifluoridates, borano phosphate esters and selenophosphates of 3-5′ linkages or 2′-5′ linkages, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. An exemplary PS ASO is illustrated below.

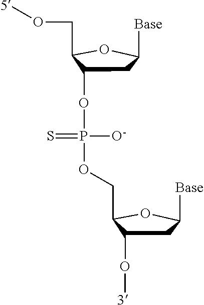

In some instances, the modification is a methyl or thiol modification such as methylphosphonate or thiolphosphonate modification. Exemplary thiolphosphonate nucleotide (left) and methylphosphonate nucleotide (right) are illustrated below.

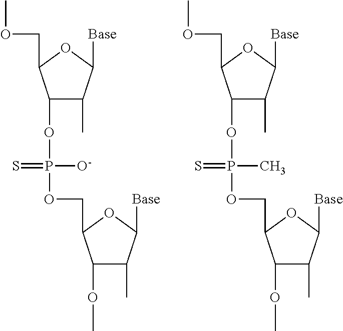

In some instances, a modified nucleotide includes, but is not limited to, 2′-fluoro N3-P5′-phosphoramidites illustrated as:

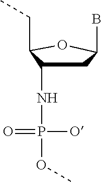

### N3′-P5′ Phosphoroamidate

In some instances, a modified nucleotide includes, but is not limited to a 5′-vinylphosphonate modified non-natural nucleotide selected from:

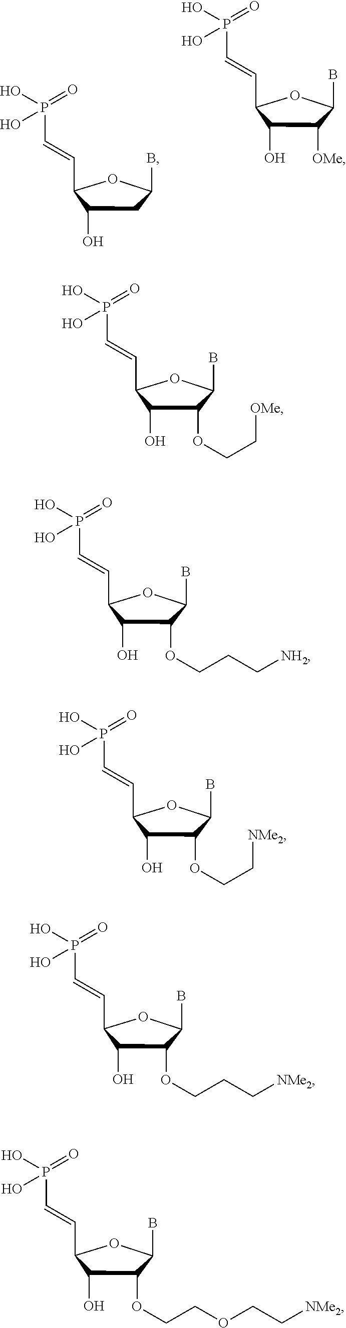

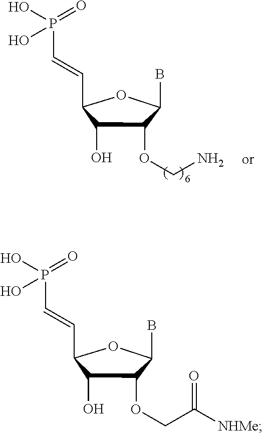

wherein B is a heterocyclic base moiety.

In some instances, a modified nucleotide includes, but is not limited to one 5′-vinylphosphonate modified non-natural nucleotide selected from:

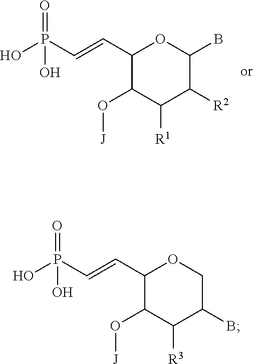

wherein B is a heterocyclic base moiety;
R¹, R², and R³ are independently selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide selected from:

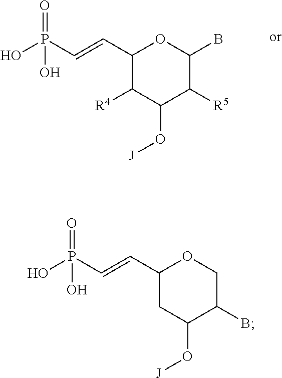

wherein B is a heterocyclic base moiety;
R⁴, and R⁵ are independently selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide selected from:

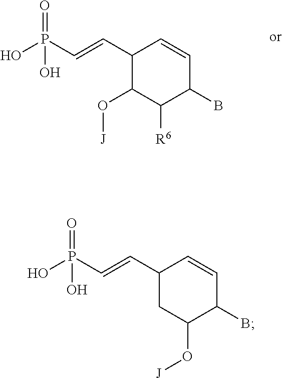

wherein B is a heterocyclic base moiety;
R⁶ is selected from hydrogen, halogen, alkyl or alkoxy; and
J is an internucleotide linking group linking to the adjacent nucleotide of the polynucleotide.

In some instances, a modified nucleotide includes, but is not limited to one 5′-vinylphosphonate modified non-natural nucleotide selected from locked nucleic acid (LNA) or ethylene nucleic acid (ENA).

In some instances, a modified nucleotide includes, but is not limited to one 5′-vinylphosphonate modified non-natural nucleotide selected from:

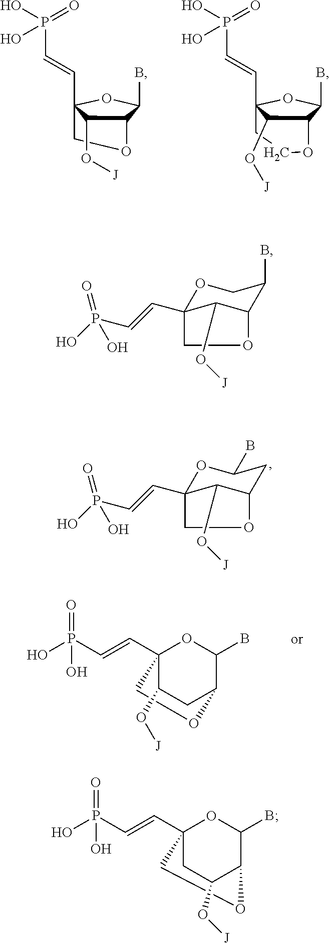

wherein B is a heterocyclic base moiety; and
J is an internucleotide linking group linking to the adjacent nucleotide of the polynucleotide.

In some instances, a modified nucleotide includes, but is not limited to one 5′-vinylphosphonate modified non-natural nucleotide selected from:

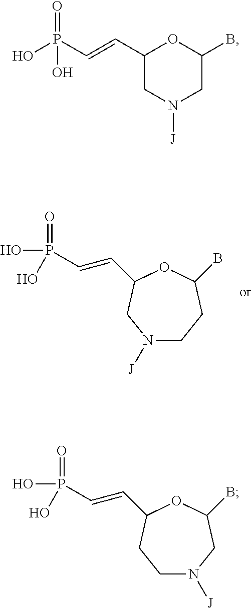

wherein B is a heterocyclic base moiety; and
J is an internucleotide linking group linking to the adjacent nucleotide of the polynucleotide.

In some instances, a modified nucleotide includes, but is not limited to one 5′-vinylphosphonate modified non-natural nucleotide selected from:

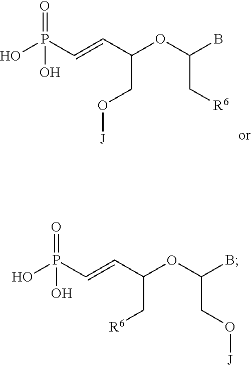

wherein B is a heterocyclic base moiety;
R⁶ is selected from hydrogen, halogen, alkyl or alkoxy; and
J is an internucleotide linking group linking to the adjacent nucleotide of the polynucleotide.

In some instances, a modified nucleotide includes, but is not limited to one 5′-vinylphosphonate modified non-natural nucleotide is:

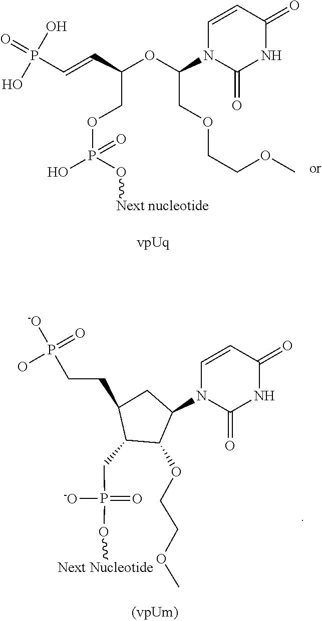

In some instances, a modified nucleotide includes, but is not limited to, hexitol nucleic acid (or 1′, 5′-anhydrohexitol nucleic acids (HNA)) illustrated as:

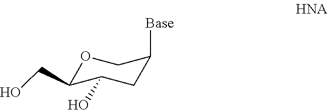

In some aspects, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some aspects, the polynucleic acid molecule comprises one or more of the artificial nucleotide analogues described herein. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the polynucleic acid molecule comprises at least one of: from about 5% to about 100% modification, from about 10% to about 100%, from about 10% to about 90%, from about 20% to about 100%, from about 10% to about 90%, from about 30% to about 100%, from about 30% to about 90%, from about 40% to about 100%, from about 40% to about 90%, from about 50% to about 100%, from about 50% to about 90%, from about 60% to about 100%, from about 60% to about 90% modification, from about 70% to about 100%, from about 70% to about 90%, from about 80% to about 100%, and from about 90% to about 100% modification.

In some cases, the polynucleic acid molecule comprises at least one of from about 10% to about 80% modification, from about 20% to about 80%, from about 20% to about 70%, from about 30% to about 80%, from about 30% to about 70%, from about 40% to about 80%, from about 40% to about 70%, from about 50% to about 80%, from about 50% to about 70%, from about 60% to about 80%, from about 60% to about 70%, and from about 70% to about 80% modification.

In some instances, the polynucleic acid molecule comprises at least one of: from about 10% to about 60% modification, from about 20% to about 60% modification, from about 30% to about 60% modification, from about 40% to about 60% modification, and from about 50% to about 60% modification.

In some cases, the polynucleic acid molecule comprises at least one of from about 10% to about 50% modification, from about 20% to about 50% modification, from about 30% to about 50% modification, and from about 40% to about 50% modification.

In some cases, the polynucleic acid molecule comprises at least one of from about 10% to about 40% modification, from about 20% to about 40% modification, and from about 30% to about 40% modification.

In some cases, the polynucleic acid molecule comprises at least one of from about 10% to about 30% modification, and from about 20% to about 30% modification.

In some cases, the polynucleic acid molecule comprises from about 10% to about 20% modification.

In some cases, the polynucleic acid molecule comprises from about 15% to about 90%, from about 20% to about 80%, from about 30% to about 70%, or from about 40% to about 60% modifications.

In additional cases, the polynucleic acid molecule comprises at least about 15%, 20%, 30%, 40%, 50%, 60%, 70%, 80%, 90%, 95%, or 99% modification.

In some aspects, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modifications.

In some instances, from about 5 to about 100% of the polynucleic acid molecule comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95% or 100% of the polynucleic acid molecule comprise the artificial nucleotide analogues described herein. In some instances, about 5% of the polynucleic acid molecule comprises the artificial nucleotide analogues described herein. In some instances, about 10% of the polynucleic acid molecule comprises the artificial nucleotide analogues described herein. In some instances, about 15% of the polynucleic acid molecule comprises the artificial nucleotide analogues described herein. In some instances, about 20% of the polynucleic acid molecule comprises the artificial nucleotide analogues described herein. In some instances, about 25% of the polynucleic acid molecule comprises the artificial nucleotide analogues described herein. In some instances, about 30% of the polynucleic acid molecule comprises the artificial nucleotide analogues described herein. In some instances, about 35% of the polynucleic acid molecule comprises the artificial nucleotide analogues described herein. In some instances, about 40% of the polynucleic acid molecule comprises the artificial nucleotide analogues described herein. In some instances, about 45% of the polynucleic acid molecule comprises the artificial nucleotide analogues described herein. In some instances, about 50% of the polynucleic acid molecule comprises the artificial nucleotide analogues described herein. In some instances, about 55% of the polynucleic acid molecule comprises the artificial nucleotide analogues described herein. In some instances, about 60% of the polynucleic acid molecule comprises the artificial nucleotide analogues described herein. In some instances, about 65% of the polynucleic acid molecule comprises the artificial nucleotide analogues described herein. In some instances, about 70% of the polynucleic acid molecule comprises the artificial nucleotide analogues described herein. In some instances, about 75% of the polynucleic acid molecule comprises the artificial nucleotide analogues described herein. In some instances, about 80% of the polynucleic acid molecule comprises the artificial nucleotide analogues described herein. In some instances, about 85% of the polynucleic acid molecule comprises the artificial nucleotide analogues described herein. In some instances, about 90% of the polynucleic acid molecule comprises the artificial nucleotide analogues described herein. In some instances, about 95% of the polynucleic acid molecule comprises the artificial nucleotide analogues described herein. In some instances, about 96% of the polynucleic acid molecule comprises the artificial nucleotide analogues described herein. In some instances, about 97% of the polynucleic acid molecule comprises the artificial nucleotide analogues described herein. In some instances, about 98% of the polynucleic acid molecule comprises the artificial nucleotide analogues described herein. In some instances, about 99% of the polynucleic acid molecule comprises the artificial nucleotide analogues described herein. In some instances, about 100% of the polynucleic acid molecule comprises the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some aspects, the polynucleic acid molecule comprises from about 1 to about 25 modifications in which the modification comprises an artificial nucleotide analogues described herein. In some aspects, the polynucleic acid molecule comprises about 1, 2, 3, 4, 5, 6, 7, 8, 9, 10, 11, 12, 13, 14, 15, 16, 17, 18, 19, 20, 21, 22, 23, 24, or 25 modification in which the modification comprises an artificial nucleotide analogue described herein.

In some aspects, a polynucleic acid molecule comprises a sense strand and antisense strand, and at least one of sense strand and antisense strands has a plurality of (e.g., two or more, three or more, four or more, five or more, six or more, seven or more, eight or more, etc.) 2′-O-methyl or 2′-deoxy-2′-fluoro modified nucleotides. In some aspects, where at least two out of the plurality of 2′-O-methyl or 2′-deoxy-2′-fluoro modified nucleotides are consecutive nucleotides. In some aspects, where consecutive 2′-O-methyl or 2′-deoxy-2′-fluoro modified nucleotides are located at the 5′-end of the sense strand and/or the antisense strand. In some aspects, where consecutive 2′-O-methyl or 2′-deoxy-2′-fluoro modified nucleotides are located at the 3′-end of the sense strand and/or the antisense strand. In some aspects, the sense strand of polynucleic acid molecule includes at least four, at least five, at least six consecutive 2′-O-methyl modified nucleotides at its 5′ end and/or 3′ end, or both. Optionally, in such embodiments, the sense strand of polynucleic acid molecule includes at least one, at least two, at least three, at least four 2′-deoxy-2′-fluoro modified nucleotides at the 3′ end of the at least four, at least five, at least six consecutive 2′-O-methyl modified nucleotides at the polynucleotides' 5′ end, or at the 5′ end of the at least four, at least five, at least six consecutive 2′-O-methyl modified nucleotides at polynucleotides' 3′ end. Also optionally, such at least two, at least three, at least four 2′-deoxy-2′-fluoro modified nucleotides are consecutive nucleotides.

In some aspects, a polynucleic acid molecule comprises a sense strand and antisense strand, and at least one of sense strand and antisense strands has 2′-O-methyl modified nucleotide located at the 5′-end of the sense strand and/or the antisense strand. In some aspects, at least one of sense strand and antisense strands has 2′-O-methyl modified nucleotide located at the 3′-end of the sense strand and/or the antisense strand. In some aspects, the 2′-O-methyl modified nucleotide located at the 5′-end of the sense strand and/or the antisense strand is a purine nucleotide. In some aspects, the 2′-O-methyl modified nucleotide located at the 5′-end of the sense strand and/or the antisense strand is a pyridine nucleotide.

In some aspects, a polynucleic acid molecule comprises a sense strand and antisense strand, and the antisense strand has two or more consecutive 2′-deoxy-2′-fluoro modified nucleotides at 5′-end. In some aspects, a polynucleic acid molecule comprises a sense strand and antisense strand, and the antisense strand has two or more consecutive 2′-O-methyl modified nucleotides at 3′-end. In some aspects, a polynucleic acid molecule comprises a sense strand and antisense strand, and the antisense strand has at least 2, 3, 4, 5, 6, or 7 consecutive 2′-O-methyl modified nucleotides.

In another embodiment, a polynucleic acid molecule described herein comprises 2′-5′ internucleotide linkages. In some instances, the 2′-5′ internucleotide linkage(s) is at the 3′-end, the 5′-end, or both of the 3′- and 5′-ends of one or both sequence strands. In addition instances, the 2′-5′ internucleotide linkage(s) is present at various other positions within one or both sequence strands, for example, about 1, 2, 3, 4, 5, 6, 7, 8, 9, 10, or more including every internucleotide linkage of a pyrimidine nucleotide in one or both strands of the polynucleic acid molecule comprise a 2′-5′ intemucleotide linkage, or about 1, 2, 3, 4, 5, 6, 7, 8, 9, 10, or more including every internucleotide linkage of a purine nucleotide in one or both strands of the polynucleic acid molecule comprise a 2′-5′ internucleotide linkage.

In some aspects, a polynucleic acid molecule is a single stranded polynucleic acid molecule that mediates RNAi activity in a cell or reconstituted in vitro system, wherein the polynucleic acid molecule comprises a single stranded polynucleotide having complementarity to a target nucleic acid sequence, and wherein one or more pyrimidine nucleotides present in the polynucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the polynucleic acid are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the polynucleic acid molecule optionally further comprising about 1 to about 4 (e.g., about 1, 2, 3, or 4) terminal 2′-deoxynucleotides at the 3′-end of the polynucleic acid molecule, wherein the terminal nucleotides further comprise one or more (e.g., 1, 2, 3, or 4) phosphorothioate intemucleotide linkages, and wherein the polynucleic acid molecule optionally further comprises a terminal phosphate group, such as a 5′-terminal phosphate group. In some instances, all internucleoside linkages of the single stranded polynucleic acid molecule comprises phosphorothioate intemucleoside linkages.

In some cases, one or more of the artificial nucleotide analogues described herein are resistant toward nucleases such as for example ribonuclease such as RNase H, deoxyribonuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, the 5′ conjugates described herein inhibit 5′-3′ exonucleolytic cleavage. In some instances, the 3′ conjugates described herein inhibit 3′-5′ exonucleolytic cleavage.

In some aspects, one or more of the artificial nucleotide analogues described herein have increased binding affinity toward their mRNA target relative to an equivalent natural polynucleic acid molecule. The one or more of the artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some cases, the increased affinity is illustrated with a lower Kd, a higher melt temperature (Tm), or a combination thereof.

In some aspects, a polynucleic acid molecule comprises a sense strand and antisense strand, and the sense strand comprises a nucleic acid of 5′-nsnsnnnnNfNfNfnnnnnnnnsnsa-3′ (lower case (n)=2′-O-Me(methyl) modified nucleotide, Nf=2′-F (fluoro) modified nucleotide; s=phosphorothioate internucleotide linkage). In some aspects, a polynucleic acid molecule comprises a sense strand and antisense strand, and the antisense strand comprises a nucleic acid of 5′-UfsNfsnnnNfnnnnnnnNfnNfnnnsusu-3′ (lower case (n)=2′-O-Me(methyl) modified nucleotide, Nf=2′-F (fluoro) modified nucleotide; s=phosphorothioate internucleotide linkage). In some aspects, a polynucleic acid molecule comprises a sense strand and antisense strand, and the sense strand comprises a nucleic acid of 5′-nsnsnnnnNfNfNfnnnnnnnnsnsa-3′ (lower case (n)=2′-O-Me(methyl) modified nucleotide, Nf=2′-F (fluoro) modified nucleotide; s=phosphorothioate internucleotide linkage) and the antisense strand comprises a nucleic acid of 5′-UfsNfsnnnNfnnnnnnnNfnNfnnnsusu-3′ (lower case (n)=2′-O-Me(methyl) modified nucleotide, Nf=2′-F (fluoro) modified nucleotide; s=phosphorothioate internucleotide linkage).

In some aspects, a polynucleic acid molecule comprises a sense strand and antisense strand, and the sense strand comprises a nucleic acid of 5′-nsnsnnnnNfNfNfnnnnnnnnsnsn-3′ (lower case (n)=2′-O-Me(methyl) modified nucleotide, Nf=2′-F (fluoro) modified nucleotide; s=phosphorothioate internucleotide linkage), and the antisense strand comprises a nucleic acid of 5′-nsNfsnnnNfnnnnnnnNfnNfnnnnnsnsn-3′ (lower case (n)=2′-O-Me(methyl) modified nucleotide, Nf=2′-F (fluoro) modified nucleotide; s=phosphorothioate internucleotide linkage). In some aspects, a polynucleic acid molecule comprises a sense strand and antisense strand, and the sense strand comprises a nucleic acid of 5′-nnnnnnnnNfNfNfnnnnnnnnnn-3′ (lower case (n)=2′-O-Me(methyl) modified nucleotide, Nf=2′-F (fluoro) modified nucleotide; and the antisense strand comprises a nucleic acid of 5′-nNfnnnNfnnnnnnnNfnNfnnnnnnn-3′ (lower case (n)=2′-O-Me(methyl) modified nucleotide, Nf=2′-F (fluoro) modified nucleotide; s=phosphorothioate internucleotide linkage). In some aspects, a polynucleic acid molecule comprises a sense strand and antisense strand, and the sense strand comprises a nucleic acid of 5′-nsnsnnnnnnNfNfNfnnnnnnnnsnsn-3′ (lower case (n)=2′-O-Me(methyl) modified nucleotide, Nf=2′-F (fluoro) modified nucleotide; s=phosphorothioate internucleotide linkage), and the antisense strand comprises a nucleic acid of 5′-nsNfsnnnNfnnnnnnnNfnNfnnnnnsnsn-3′ (lower case (n)=2′-O-Me(methyl) modified nucleotide, Nf=2′-F (fluoro) modified nucleotide; s=phosphorothioate internucleotide linkage). In some aspects, a polynucleic acid molecule comprises a sense strand and antisense strand, and the sense strand comprises a nucleic acid of 5′-nnnnnnnnNfNfNfnnnnnnnnnn-3′ (lower case (n)=2′-O-Me(methyl) modified nucleotide, Nf=2′-F (fluoro) modified nucleotide; and the antisense strand comprises a nucleic acid of 5′-VpUqNfnnnNfnnnnnnnNfnNfnnnnnnn-3′ (lower case (n)=2′-O-Me(methyl) modified nucleotide, Nf=2′-F (fluoro) modified nucleotide; s=phosphorothioate internucleotide linkage). In some aspects, a polynucleic acid molecule comprises a sense strand and antisense strand, and the sense strand comprises a nucleic acid of 5′-nsnsnnnnnnNfNfNfnnnnnnnnsnsn-3′ (lower case (n)=2′-O-Me(methyl) modified nucleotide, Nf=2′-F (fluoro) modified nucleotide; s=phosphorothioate internucleotide linkage), and the antisense strand comprises a nucleic acid of 5′-VpUqsNfsnnnNfnnnnnnnNfnNfinnnnsnsn-3′ (lower case (n)=2′-O-Me(methyl) modified nucleotide, Nf=2′-F (fluoro) modified nucleotide; s=phosphorothioate internucleotide linkage).

In some aspects, a polynucleic acid molecule described herein is a chirally pure (or stereo pure) polynucleic acid molecule, or a polynucleic acid molecule comprising a single enantiomer. In some instances, the polynucleic acid molecule comprises L-nucleotide. In some instances, the polynucleic acid molecule comprises D-nucleotides. In some instance, a polynucleic acid molecule composition comprises less than 30%, 25%, 20%, 15%, 10%, 5%, 4%, 3%, 2%, 10%, or less of its mirror enantiomer. In some cases, a polynucleic acid molecule composition comprises less than 30%, 25%, 20%, 15%, 10%, 5%, 4%, 3%, 2%, 1%, or less of a racemic mixture. In some instances, the polynucleic acid molecule is a polynucleic acid molecule described in: U.S. Patent Publication Nos: 2014/194610 and 2015/211006; and PCT Publication No: WO2015107425.

In some aspects, a polynucleic acid molecule described herein is further modified to include an aptamer conjugating moiety. In some instances, the aptamer conjugating moiety is a DNA aptamer conjugating moiety. In some instances, the aptamer conjugating moiety is Alphamer (Centauri Therapeutics), which comprises an aptamer portion that recognizes a specific cell-surface target and a portion that presents a specific epitope for attaching to circulating antibodies. In some instance, a polynucleic acid molecule described herein is further modified to include an aptamer conjugating moiety as described in: U.S. Pat. Nos. 8,604,184, 8,591,910, and 7,850,975.

In additional embodiments, a polynucleic acid molecule described herein is modified to increase its stability. In some embodiment, the polynucleic acid molecule is RNA (e.g., siRNA). In some instances, the polynucleic acid molecule is modified by one or more of the modifications described above to increase its stability. In some cases, the polynucleic acid molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modification or by a locked or bridged ribose conformation (e.g., LNA or ENA). In some cases, the polynucleic acid molecule is modified by 2′-O-methyl and/or 2′-O-methoxyethyl ribose. In some cases, the polynucleic acid molecule also includes morpholinos, PNAs, HNA, methylphosphonate nucleotides, thiolphosphonate nucleotides, and/or 2′-fluoro N3-P5′-phosphoramidites to increase its stability. In some instances, the polynucleic acid molecule is a chirally pure (or stereo pure) polynucleic acid molecule. In some instances, the chirally pure (or stereo pure) polynucleic acid molecule is modified to increase its stability. Suitable modifications to the RNA to increase stability for delivery will be apparent to the skilled person.

In some aspects, an asymmetric duplex is a polynucleic acid molecule having two separate strands comprising a sense strand and an antisense strand, wherein the sense strand comprises fewer nucleotides than the antisense strand to the extent that the sense strand has enough complimentary nucleotides to base pair with the antisense strand and form a duplex. For example, an asymmetric duplex polynucleic acid molecule comprises an antisense region having length sufficient to mediate RNAi in a cell or in vitro system (e.g., about 19 to about 22 nucleotides) and a sense strand having about 3 to about 18 nucleotides that are complementary to the antisense strand.

### Polynucleic Acid Molecule Synthesis

In some aspects, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex formed between the polynucleic acid molecule and target nucleic acids. Exemplary methods include those described in: U.S. Pat. Nos. 5,142,047; 5,185,444; 5,889,136; 6,008,400; and 6,111,086; PCT Publication No. WO2009099942; or European Publication NO. 1579015. Additional exemplary methods include those described in: Griffey et al., “2′-O-aminopropyl ribonucleotides: a zwitterionic modification that enhances the exonuclease resistance and biological activity of antisense oligonucleotides,” J Med. Chem. 39 (26):5100-5109 (1997)); Obika, et al. “Synthesis of 2′-0,4′-C-methyleneuridine and -cytidine. Novel bicyclic nucleosides having a fixed C₃, -endo sugar puckering”. Tetrahedron Letters 38 (50): 8735 (1997); Koizumi, M. “ENA oligonucleotides as therapeutics”. Current opinion in molecular therapeutics 8 (2): 144-149 (2006); and Abramova et al., “Novel oligonucleotide analogues based on morpholino nucleoside subunits-antisense technologies: new chemical possibilities,” Indian Journal of Chemistry 48B:1721-1726 (2009). Alternatively, the polynucleic acid molecule is produced biologically using an expression vector into which a polynucleic acid molecule has been subcloned in an antisense orientation (i.e., RNA transcribed from the inserted polynucleic acid molecule will be of an antisense orientation to a target polynucleic acid molecule of interest).

In some aspects, a polynucleic acid molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, a polynucleic acid molecule is also assembled from two distinct nucleic acid strands or fragments wherein one fragment includes the sense region and the second fragment includes the antisense region of the molecule.

### Polynucleic Acid Molecule Conjugates

In some aspects, a polynucleic acid molecule (B) is further conjugated to a polypeptide (A) for delivery to a site of interest. In some instances, at least one polypeptide A is conjugated to at least one B. In some instances, the at least one polypeptide A is conjugated to the at least one B to form an A-B conjugate. In some aspects, at least one A is conjugated to the 5′ terminus of B, the 3′ terminus of B, an internal site on B, or in any combinations thereof. In some instances, the at least one polypeptide A is conjugated to at least two B. In some instances, the at least one polypeptide A is conjugated to at least 2, 3, 4, 5, 6, 7, 8, or more B.

In some cases, a polynucleic acid molecule is conjugated to a polypeptide (A) and optionally a polymeric moiety (C). In some aspects, at least one polypeptide A is conjugated at one terminus of at least one B while at least one C is conjugated at the opposite terminus of the at least one B to form an A-B-C conjugate. In some instances, at least one polypeptide A is conjugated at one terminus of the at least one B while at least one of C is conjugated at an internal site on the at least one B. In some instances, at least one polypeptide A is conjugated directly to the at least one C. In some instances, the at least one B is conjugated indirectly to the at least one polypeptide A via the at least one C to form an A-C-B conjugate.

In some instances, at least one B and/or at least one C, and optionally at least one D are conjugated to at least one polypeptide A. In some instances, the at least one B is conjugated at a terminus (e.g., a 5′ terminus or a 3′ terminus) to the at least one polypeptide A or are conjugated via an internal site to the at least one polypeptide A. In some cases, the at least one C is conjugated either directly to the at least one polypeptide A or indirectly via the at least one B. If indirectly via the at least one B, the at least one C is conjugated either at the same terminus as the at least one polypeptide A on B, at opposing terminus from the at least one polypeptide A, or independently at an internal site. In some instances, at least one additional polypeptide A is further conjugated to the at least one polypeptide A, to B, or to C. In additional instances, the at least one D is optionally conjugated either directly or indirectly to the at least one polypeptide A, to the at least one B, or to the at least one C. If directly to the at least one polypeptide A, the at least one D is also optionally conjugated to the at least one B to form an A-D-B conjugate or is optionally conjugated to the at least one B and the at least one C to form an A-D-B-C conjugate. In some instances, the at least one D is directly conjugated to the at least one polypeptide A and indirectly to the at least one B and the at least one C to form a D-A-B-C conjugate. If indirectly to the at least one polypeptide A, the at least one D is also optionally conjugated to the at least one B to form an A-B-D conjugate or is optionally conjugated to the at least one B and the at least one C to form an A-B-D-C conjugate. In some instances, at least one additional D is further conjugated to the at least one polypeptide A, to B, or to C.

### Binding Moiety

In some aspects, the binding moiety is a polypeptide. In some instances, the polypeptide is an antibody or its fragment thereof. In some cases, the fragment is a binding fragment. In some instances, the antibody or antigen binding fragment thereof comprises a humanized antibody or antigen binding fragment thereof, murine antibody or antigen binding fragment thereof, chimeric antibody or antigen binding fragment thereof, monoclonal antibody or antigen binding fragment thereof, a binding fragment having a light chain domain and a heavy chain domain, a binding fragment having two light chain domains and two heavy chain domains, a binding fragment having two or more light chain domains and heavy chain domains, monovalent Fab, Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, VHH, camelid antibody or antigen binding fragment thereof, bispecific antibody or biding fragment thereof, or a chemically modified derivative thereof. In some instances, the antibody or antigen-binding fragment thereof is a single-arm antibody, Fab-Fc fusion antibody, or VHH-Fc fusion antibody.

In some aspects, the binding moiety is a bispecific antibody or antigen binding fragment thereof. In some instances, the bispecific antibody is a trifunctional antibody or a bispecific mini-antibody. In some cases, the bispecific antibody is a trifunctional antibody. In some instances, the trifunctional antibody is a full-length monoclonal antibody comprising binding sites for two different antigens.

In some cases, the bispecific antibody is a bispecific mini-antibody. In some instances, the bispecific mini-antibody comprises divalent Fab₂, F(ab)′₃ fragments, bis-scFv, (scFv)₂, diabody, minibody, triabody, tetrabody or a bi-specific T-cell engager (BiTE). In some aspects, the bi-specific T-cell engager is a fusion protein that contains two single-chain variable fragments (scFvs) in which the two scFvs target epitopes of two different antigens.

In some aspects, the binding moiety is a bispecific mini-antibody. In some instances, A is a bispecific Fab₂. In some instances, binding moiety is a bispecific F(ab)′₃ fragment. In some cases, binding moiety is a bispecific bis-scFv. In some cases, binding moiety is a bispecific (scFv)₂. In some aspects, binding moiety is a bispecific diabody. In some aspects, binding moiety is a bispecific minibody. In some aspects, binding moiety is a bispecific triabody. In other embodiments, binding moiety is a bispecific tetrabody. In other embodiments, binding moiety is a bi-specific T-cell engager (BiTE).

In some aspects, the binding moiety is a trispecific antibody. In some instances, the trispecific antibody comprises F(ab)′₃ fragments or a triabody. In some instances, binding moiety is a trispecific F(ab)′₃ fragment. In some cases, binding moiety is a triabody. In some aspects, A is a trispecific antibody as described in Dimas, et al., “Development of a trispecific antibody designed to simultaneously and efficiently target three different antigens on tumor cells,” Mol. Pharmaceutics, 12(9): 3490-3501 (2015).

In some embodiments, the binding moiety is a multicyclic peptide. In some embodiments, the binding moiety is a bicyclic or tricyclic peptide. In some embodiments, the binding moiety is a bicyclic or tricyclic peptide that specifically binds to a transferrin receptor (TfR), preferably, specifically binds to transferrin receptor 1 (TfR1), or more preferably, specifically binds to human transferrin receptor 1 (TfR1) (or human CD71). In some aspects, the binding moiety is an antibody or antigen binding fragment thereof that recognizes a cell surface protein. In some instances, the binding moiety is an antibody or antigen binding fragment thereof that recognizes a cell surface protein on a muscle cell. In some cases, the binding moiety is an antibody or antigen binding fragment thereof that recognizes a cell surface protein on a skeletal muscle cell. In some cases, the binding moiety is an antibody or antigen binding fragment thereof that recognizes a cell surface protein on a cardiac muscle cell. In some cases, the binding moiety is an antibody or antigen-binding fragment thereof for extrahepatic delivery. In some cases, the binding moiety is an antibody or antigen-binding fragment thereof for delivery to a muscle cell. In some cases, the binding moiety is an antibody or antigen-binding fragment thereof for delivery to a cardiac muscle cell.

In some aspects, exemplary antibodies include, but are not limited to, an anti-myosin antibody, an anti-transferrin receptor antibody, and an antibody that recognizes Muscle-Specific kinase (MuSK). In some instances, the antibody is an anti-transferrin receptor (anti-CD71) antibody.

In some aspects, where the antibody is an anti-transferrin receptor (anti-CD71) antibody, the anti-transferrin antibody specifically binds to a transferrin receptor (TfR), preferably, specifically binds to transferrin receptor 1 (TfRT), or more preferably, specifically binds to human transferrin receptor 1 (TfRT) (or human CD71).

In some instances, the anti-transferrin receptor antibody comprises a variable heavy chain (VH) region and a variable light chain (VL) region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X2 is selected from Q or E; and HCDR3 sequence comprising SEQ ID NO: 245.

In some aspects, the VH region of the anti-transferrin receptor antibody comprises HCDR1, HCDR2, and HCDR3 sequences selected from Table 1.

TABLE 1

| Name | HCDR1 | SEQ ID NO: | HCDR2 | SEQ ID NO: | HCDR3 | SEQ ID NO: |
|---|---|---|---|---|---|---|
| 13E4_VH1 | YTFTNYWMH | 240 | EINPINGRSNYAQKFQG | 242 | GTRAMHY | 245 |
| 13E4_VH2* | YTFTNYWMH | 240 | EINPINGRSNYAEKFQG | 243 | GTRAMHY | 245 |
| 13E4_VH3 | YTFTNYWMH | 240 | EINPIQGRSNYAEKFQG | 244 | GTRAMHY | 245 |

*13E4_VH2 shares the same HCDR1, HCDR2, and HCDR3 sequences with anti-transferrin receptor
antibody 13E4_VH4

In some aspects, the VH region comprises HCDR1 sequence comprising SEQ ID NO: 240; HCDR2 sequence comprising SEQ ID NO: 242, 243, or 244; and HCDR3 sequence comprising SEQ ID NO: 245. In some instances, the VH region comprises HCDR1 sequence comprising SEQ ID NO: 240, HCDR2 sequence comprising SEQ ID NO: 242, and HCDR3 sequence comprising SEQ ID NO: 245. In some instances, the VH region comprises HCDR1 sequence comprising SEQ ID NO: 240, HCDR2 sequence comprising SEQ ID NO: 243, and HCDR3 sequence comprising SEQ ID NO: 245. In some instances, the VH region comprises HCDR1 sequence comprising SEQ ID NO: 240, HCDR2 sequence comprising SEQ ID NO: 244, and HCDR3 sequence comprising SEQ ID NO: 245.

In some aspects, the VL region of the anti-transferrin receptor antibody comprises LCDR1 sequence RTSENIYX₃NLA (SEQ ID NO: 246), LCDR2 sequence AX₄TNLAX₅ (SEQ ID NO: 249), and LCDR3 sequence QHFWGTPLTX₆ (SEQ ID NO: 253), wherein X₃ is selected from N or S, X₄ is selected from A or G, X₅ is selected from D or E, and X₆ is present or absence, and if present, is F.

In some aspects, the VL region of the anti-transferrin receptor antibody comprises LCDR1, LCDR2, and LCDR3 sequences selected from Table 2.

TABLE 2

| Name | LCDR1 | SEQ ID NO: | LCDR2 | SEQ ID NO: | LCDR3 | SEQ ID NO: |
|---|---|---|---|---|---|---|
| 13E4_VL1* | RTSENIYNNLA | 247 | AATNLAD | 250 | QHFWGTPLT | 254 |
| 13E4_VL3 | RTSENIYNNLA | 247 | AATNLAE | 251 | QHFWGTPLTF | 255 |
| 13E4_VL4 | RTSENIYSNLA | 248 | AGTNLAD | 252 | QHFWGTPLTF | 255 |

*13E4_VL1 shares the same LCDR1, LCDR2, and LCDR3 sequences with anti-transferrin receptor
antibody 13E4_VL2

In some instances, the VL region comprises LCDR1 sequence RTSENIYX₃NLA (SEQ ID NO: 246), LCDR2 sequence comprising SEQ ID NO: 250, 251, or 252, and LCDR3 sequence comprising SEQ ID NO: 254 or 255, wherein X₃ is selected from N or S.

In some instances, the VL region comprises LCDR1 sequence comprising SEQ ID NO: 247 or 248, LCDR2 sequence AX₄TNLAX₅ (SEQ ID NO: 249), and LCDR3 sequence comprising SEQ ID NO: 254 or 255, wherein X₄ is selected from A or G, and X₅ is selected from D or E.

In some instances, the VL region comprises LCDR1 sequence comprising SEQ ID NO: 247 or 248, LCDR2 sequence SEQ ID NO: 250, 251, or 252, and LCDR3 sequence QHFWGTPLTX₆ (SEQ ID NO: 253), wherein X₆ is present or absence, and if present, is F.

In some instances, the VL region comprises LCDR1 sequence comprising SEQ ID NO: 247, LCDR2 sequence AX₄TNLAX₅ (SEQ ID NO: 249), and LCDR3 sequence QHFWGTPLTX₆ (SEQ ID NO: 253), wherein X₅ is selected from D or E and X₆ is present or absence, and if present, is F.

In some instances, the VL region comprises LCDR1 sequence comprising SEQ ID NO: 247, LCDR2 sequence comprising SEQ ID NO: 250, and LCDR3 sequence comprising SEQ ID NO: 254.

In some instances, the VL region comprises LCDR1 sequence comprising SEQ ID NO: 247, LCDR2 sequence comprising SEQ ID NO: 251, and LCDR3 sequence comprising SEQ ID NO: 255.

In some instances, the VL region comprises LCDR1 sequence comprising SEQ ID NO: 248, LCDR2 sequence comprising SEQ ID NO: 252, and LCDR3 sequence comprising SEQ ID NO: 255.

In some aspects, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence RTSENIYX₃NLA (SEQ ID NO: 246), LCDR2 sequence AX₄TNLAX₅ (SEQ ID NO: 249), and LCDR3 sequence QHFWGTPLTX₆ (SEQ ID NO: 253), wherein X₃ is selected from N or S, X₄ is selected from A or G, X₅ is selected from D or E, and X₆ is present or absence, and if present, is F.

In some instances, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence RTSENIYX₃NLA (SEQ ID NO: 246), LCDR2 sequence comprising SEQ ID NO: 250, 251, or 252, and LCDR3 sequence comprising SEQ ID NO: 254 or 255, wherein X₃ is selected from N or S.

In some instances, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence comprising SEQ ID NO: 247 or 248, LCDR2 sequence AX₄TNLAX₅ (SEQ ID NO: 249), and LCDR3 sequence comprising SEQ ID NO: 254 or 255, wherein X₄ is selected from A or G, and X₅ is selected from D or E.

In some instances, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence comprising SEQ ID NO: 247 or 248, LCDR2 sequence SEQ ID NO: 250, 251, or 252, and LCDR3 sequence QHFWGTPLTX₆ (SEQ ID NO: 253), wherein X₆ is present or absence, and if present, is F.

In some instances, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence comprising SEQ ID NO: 247, LCDR2 sequence AX₄TNLAX₅ (SEQ ID NO: 249), and LCDR3 sequence QHFWGTPLTX₆ (SEQ ID NO: 253), wherein X₅ is selected from D or E and X₆ is present or absence, and if present, is F.

In some instances, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence comprising SEQ ID NO: 247, LCDR2 sequence comprising SEQ ID NO: 250, and LCDR3 sequence comprising SEQ ID NO: 254.

In some instances, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence comprising SEQ ID NO: 247, LCDR2 sequence comprising SEQ ID NO: 251, and LCDR3 sequence comprising SEQ ID NO: 255.

In some instances, the anti-transferrin receptor antibody comprises a VH region and a VL region, wherein the VH region comprises HCDR1 sequence comprising SEQ ID NO: 240; HCDR2 sequence EINPIXiGRSNYAX2KFQG (SEQ ID NO: 241), wherein X₁ is selected from N or Q and X₂ is selected from Q or E; and HCDR3 sequence comprising SEQ ID NO: 245; and the VL region comprises LCDR1 sequence comprising SEQ ID NO: 248, LCDR2 sequence comprising SEQ ID NO: 252, and LCDR3 sequence comprising SEQ ID NO: 255.

In some instances, the anti-transferrin receptor antibody comprises a VH region and a VL region, in which the VH region comprises HCDR1 sequence comprising SEQ ID NO: 240, HCDR2 sequence comprising SEQ ID NO: 242, and HCDR3 sequence comprising SEQ ID NO: 245; and the VL region comprises LCDR1 sequence RTSENIYX₃NLA (SEQ ID NO: 246), LCDR2 sequence comprising SEQ ID NO: 250, 251, or 252, and LCDR3 sequence comprising SEQ ID NO: 254 or 255, wherein X₃ is selected from N or S.

In some instances, the anti-transferrin receptor antibody comprises a VH region and a VL region, in which the VH region comprises HCDR1 sequence comprising SEQ ID NO: 240, HCDR2 sequence comprising SEQ ID NO: 242, and HCDR3 sequence comprising SEQ ID NO: 245; and the VL region comprises LCDR1 sequence comprising SEQ ID NO: 247 or 248, LCDR2 sequence AX₄TNLAX₅ (SEQ ID NO: 249), and LCDR3 sequence comprising SEQ ID NO: 254 or 255, wherein X₄ is selected from A or G, and X₅ is selected from D or E.

In some instances, the anti-transferrin receptor antibody comprises a VH region and a VL region, in which the VH region comprises HCDR1 sequence comprising SEQ ID NO: 240, HCDR2 sequence comprising SEQ ID NO: 2, and HCDR3 sequence comprising SEQ ID NO: 245; and the VL region comprises LCDR1 sequence comprising SEQ ID NO: 247 or 248, LCDR2 sequence SEQ ID NO: 250, 251, or 252, and LCDR3 sequence QHFWGTPLTX₆ (SEQ ID NO: 253), wherein X₆ is present or absence, and if present, is F.

In some instances, the anti-transferrin receptor antibody comprises a VH region and a VL region, in which the VH region comprises HCDR1 sequence comprising SEQ ID NO: 240, HCDR2 sequence comprising SEQ ID NO: 242, and HCDR3 sequence comprising SEQ ID NO: 245; and the VL region comprises LCDR1 sequence comprising SEQ ID NO: 247, LCDR2 sequence AX₄TNLAX₅ (SEQ ID NO: 249), and LCDR3 sequence QHFWGTPLTX₆ (SEQ ID NO: 253), wherein X₅ is selected from D or E and X₆ is present or absence, and if present, is F.

In some instances, the anti-transferrin receptor antibody comprises a VH region and a VL region, in which the VH region comprises HCDR1 sequence comprising SEQ ID NO: 240, HCDR2 sequence comprising SEQ ID NO: 242, and HCDR3 sequence comprising SEQ ID NO: 245; and the VL region comprises LCDR1 sequence comprising SEQ ID NO: 247, LCDR2 sequence comprising SEQ ID NO: 250, and LCDR3 sequence comprising SEQ ID NO: 254.

In some instances, the anti-transferrin receptor antibody comprises a VH region and a VL region, in which the VH region comprises HCDR1 sequence comprising SEQ ID NO: 240, HCDR2 sequence comprising SEQ ID NO: 242, and HCDR3 sequence comprising SEQ ID NO: 245; and the VL region comprises LCDR1 sequence comprising SEQ ID NO: 247, LCDR2 sequence comprising SEQ ID NO: 242, and LCDR3 sequence comprising SEQ ID NO: 255.

In some instances, the anti-transferrin receptor antibody comprises a VH region and a VL region, in which the VH region comprises HCDR1 sequence comprising SEQ ID NO: 240, HCDR2 sequence comprising SEQ ID NO: 242, and HCDR3 sequence comprising SEQ ID NO: 245; and the VL region comprises LCDR1 sequence comprising SEQ ID NO: 248, LCDR2 sequence comprising SEQ ID NO: 252, and LCDR3 sequence comprising SEQ ID NO: 255.

In some instances, the anti-transferrin receptor antibody comprises a VH region and a VL region, in which the VH region comprises HCDR1 sequence comprising SEQ ID NO: 240, HCDR2 sequence comprising SEQ ID NO: 243, and HCDR3 sequence comprising SEQ ID NO: 245; and the VL region comprises LCDR1 sequence RTSENIYX₃NLA (SEQ ID NO: 246), LCDR2 sequence comprising SEQ ID NO: 250, 251, or 252, and LCDR3 sequence comprising SEQ ID NO: 254 or 255, wherein X₃ is selected from N or S.

In some instances, the anti-transferrin receptor antibody comprises a VH region and a VL region, in which the VH region comprises HCDR1 sequence comprising SEQ ID NO: 240, HCDR2 sequence comprising SEQ ID NO: 243, and HCDR3 sequence comprising SEQ ID NO: 245; and the VL region comprises LCDR1 sequence comprising SEQ ID NO: 247 or 248, LCDR2 sequence AX₄TNLAX₅ (SEQ ID NO: 249), and LCDR3 sequence comprising SEQ ID NO: 254 or 255, wherein X₄ is selected from A or G, and X₅ is selected from D or E.

In some instances, the anti-transferrin receptor antibody comprises a VH region and a VL region, in which the VH region comprises HCDR1 sequence comprising SEQ ID NO: 240, HCDR2 sequence comprising SEQ ID NO: 243, and HCDR3 sequence comprising SEQ ID NO: 245; and the VL region comprises LCDR1 sequence comprising SEQ ID NO: 247 or 248, LCDR2 sequence SEQ ID NO: 250, 251, or 252, and LCDR3 sequence QHFWGTPLTX₆ (SEQ ID NO: 253), wherein X₆ is present or absence, and if present, is F.

In some instances, the anti-transferrin receptor antibody comprises a VH region and a VL region, in which the VH region comprises HCDR1 sequence comprising SEQ ID NO: 240, HCDR2 sequence comprising SEQ ID NO: 243, and HCDR3 sequence comprising SEQ ID NO: 245; and the VL region comprises LCDR1 sequence comprising SEQ ID NO: 247, LCDR2 sequence AX₄TNLAX₅ (SEQ ID NO: 249), and LCDR3 sequence QHFWGTPLTX₆ (SEQ ID NO: 253), wherein X₅ is selected from D or E and X₆ is present or absence, and if present, is F.

In some instances, the anti-transferrin receptor antibody comprises a VH region and a VL region, in which the VH region comprises HCDR1 sequence comprising SEQ ID NO: 240, HCDR2 sequence comprising SEQ ID NO: 243, and HCDR3 sequence comprising SEQ ID NO: 245; and the VL region comprises LCDR1 sequence comprising SEQ ID NO: 247, LCDR2 sequence comprising SEQ ID NO: 250, and LCDR3 sequence comprising SEQ ID NO: 254.

In some instances, the anti-transferrin receptor antibody comprises a VH region and a VL region, in which the VH region comprises HCDR1 sequence comprising SEQ ID NO: 240, HCDR2 sequence comprising SEQ ID NO: 243, and HCDR3 sequence comprising SEQ ID NO: 245; and the VL region comprises LCDR1 sequence comprising SEQ ID NO: 247, LCDR2 sequence comprising SEQ ID NO: 251, and LCDR3 sequence comprising SEQ ID NO: 255.

In some instances, the anti-transferrin receptor antibody comprises a VH region and a VL region, in which the VH region comprises HCDR1 sequence comprising SEQ ID NO: 240, HCDR2 sequence comprising SEQ ID NO: 243, and HCDR3 sequence comprising SEQ ID NO: 245; and the VL region comprises LCDR1 sequence comprising SEQ ID NO: 248, LCDR2 sequence comprising SEQ ID NO: 252, and LCDR3 sequence comprising SEQ ID NO: 255.

In some instances, the anti-transferrin receptor antibody comprises a VH region and a VL region, in which the VH region comprises HCDR1 sequence comprising SEQ ID NO: 240, HCDR2 sequence comprising SEQ ID NO: 244, and HCDR3 sequence comprising SEQ ID NO: 245; and the VL region comprises LCDR1 sequence RTSENIYX₃NLA (SEQ ID NO: 246), LCDR2 sequence comprising SEQ ID NO: 250, 251, or 252, and LCDR3 sequence comprising SEQ ID NO: 254 or 255, wherein X₃ is selected from N or S.

In some instances, the anti-transferrin receptor antibody comprises a VH region and a VL region, in which the VH region comprises HCDR1 sequence comprising SEQ ID NO: 240, HCDR2 sequence comprising SEQ ID NO: 244, and HCDR3 sequence comprising SEQ ID NO: 245; and the VL region comprises LCDR1 sequence comprising SEQ ID NO: 247 or 248, LCDR2 sequence AX₄TNLAX₅ (SEQ ID NO: 249), and LCDR3 sequence comprising SEQ ID NO: 254 or 255, wherein X₄ is selected from A or G, and X₅ is selected from D or E.

In some instances, the anti-transferrin receptor antibody comprises a VH region and a VL region, in which the VH region comprises HCDR1 sequence comprising SEQ ID NO: 240, HCDR2 sequence comprising SEQ ID NO: 244, and HCDR3 sequence comprising SEQ ID NO: 245; and the VL region comprises LCDR1 sequence comprising SEQ ID NO: 247 or 248, LCDR2 sequence SEQ ID NO: 250, 251, or 252, and LCDR3 sequence QHFWGTPLTX₆ (SEQ ID NO: 253), wherein X₆ is present or absence, and if present, is F.

In some instances, the anti-transferrin receptor antibody comprises a VH region and a VL region, in which the VH region comprises HCDR1 sequence comprising SEQ ID NO: 240, HCDR2 sequence comprising SEQ ID NO: 244, and HCDR3 sequence comprising SEQ ID NO: 245 and the VL region comprises LCDR1 sequence comprising SEQ ID NO: 247, LCDR2 sequence AX₄TNLAX₅ (SEQ ID NO: 249), and LCDR3 sequence QHFWGTPLTX₆ (SEQ ID NO: 253), wherein X₅ is selected from D or E and X₆ is present or absence, and if present, is F.

In some instances, the anti-transferrin receptor antibody comprises a VH region and a VL region, in which the VH region comprises HCDR1 sequence comprising SEQ ID NO: 240, HCDR2 sequence comprising SEQ ID NO: 244, and HCDR3 sequence comprising SEQ ID NO: 245; and the VL region comprises LCDR1 sequence comprising SEQ ID NO: 247, LCDR2 sequence comprising SEQ ID NO: 250, and LCDR3 sequence comprising SEQ ID NO: 254.

In some instances, the anti-transferrin receptor antibody comprises a VH region and a VL region, in which the VH region comprises HCDR1 sequence comprising SEQ ID NO: 240, HCDR2 sequence comprising SEQ ID NO: 244, and HCDR3 sequence comprising SEQ ID NO: 245; and the VL region comprises LCDR1 sequence comprising SEQ ID NO: 247, LCDR2 sequence comprising SEQ ID NO: 251, and LCDR3 sequence comprising SEQ ID NO: 255.

In some instances, the anti-transferrin receptor antibody comprises a VH region and a VL region, in which the VH region comprises HCDR1 sequence comprising SEQ ID NO: 240, HCDR2 sequence comprising SEQ ID NO: 244, and HCDR3 sequence comprising SEQ ID NO: 245; and the VL region comprises LCDR1 sequence comprising SEQ ID NO: 248, LCDR2 sequence comprising SEQ ID NO: 252, and LCDR3 sequence comprising SEQ ID NO: 255.

In some aspects, the anti-transferrin receptor antibody comprises a VH region and a VL region in which the sequence of the VH region comprises about 80%, 85%, 90%, 95%, 96%, 97%, 98%, 99%, or 100% sequence identity to SEQ ID NOs: 256-260 and the sequence of the VL region comprises about 80%, 85%, 90%, 95%, 96%, 97%, 98%, 99%, or 100% sequence identity to SEQ ID NOs: 261-265.

In some aspects, the VH region comprises a sequence selected from SEQ ID NOs: 293-260 (Table 3) and the VL region comprises a sequence selected from SEQ ID NOs: 261-264 (Table 4). The underlined regions in Table 3 and Table 4 denote the respective CDR1, CDR2, or CDR3 sequence.

TABLE 3

| NAME | VH SEQUENCE | SEQ ID NO: |
|---|---|---|
| 13E4_VH1 | QVQLVQSGAEVKKPGASVKVSCKASGYTFTNYWMHWVRQAPGQ | 256 |
| | GLEWMGEINPINGRSNYAQKFQGRVTLTVDTSISTAYMELSRLRSD | |
| | DTAVYYCARGTRAMHYWGQGTLVTVSS | |
| 13E4_VH2 | QVQLVQSGAEVKKPGASVKVSCKASGYTFTNYWMHWVRQAPGQ | 257 |
| | GLEWIGEINPINGRSNYAEKFQGRVTLTVDTSSSTAYMELSRLRSD | |
| | DTAVYYCARGTRAMHYWGQGTLVTVSS | |
| 13E4_VH3 | QVQLVQSGAEVKKPGASVKVSCKASGYTFTNYWMHWVRQAPGQ | 258 |
| | GLEWMGEINPIQGRSNYAEKFQGRVTLTVDTSSSTAYMELSSLRSE | |
| | DTATYYCARGTRAMHYWGQGTLVTVSS | |
| 13E4_VH4 | QVQLVQSGAEVKKPGASVKVSCKASGYTFTNYWMHWVRQAPGQ | 259 |
| | GLEWMGEINPINGRSNYAEKFQGRVTLTVDTSSSTAYMELSSLRSE | |
| | DTATYYCARGTRAMHYWGQGTLVTVSS | |
| 13E4_VH | QVQLQQPGAELVKPGASVKLSCKASGYTFTNYWMHWVKQRPGQ | 260 |
| | GLEWIGEINPINGRSNYGERFKTKATLTVDKSSSTAYMQLSSLTSE | |
| | DSAVYYCARGTRAMHYWGQGTSVTVSS | |

TABLE 4

| NAME | VL SEQUENCE | SEQ ID NO: |
|---|---|---|
| 13E4_VL1 | DIQMTQSPSSLSASVGDRVTITCRTSENIYNNLAWYQQKPGKSPKLLIYAA | 261 |
| | TNLADGVPSRFSGSGSGTDYTLTISSLQPEDFATYYCQHFWGTPLTFGGGT | |
| | KVEIK | |
| 13E4_VL2 | DIQMTQSPSSLSASVGDRVTITCRTSENIYNNLAWYQQKPGKAPKLLIYA | 262 |
| | ATNLADGVPSRFSGSGSGTDYTLTISSLQPEDFATYYCQHFWGTPLTFGG | |
| | GTKVEIK | |
| 13E4_VL3 | DIQMTQSPSSLSASVGDRVTITCRTSENIYNNLAWYQQKPGKAPKLLIYA | 263 |
| | ATNLAEGVPSRFSGSGSGTDYTLTISSLQPEDFATYYCQHFWGTPLTFGGG | |
| | TKVEIK | |
| 13E4_VL4 | DIQMTQSPSSLSASVGDRVTITCRTSENIYSNLAWYQQKPGKAPKLLIYAG | 264 |
| | TNLADGVPSRFSGSGSGTDYTLTISSLQPEDFANYYCQHFWGTPLTFGGG | |
| | TKVEIK | |
| 13E4_VL | DIQMTQSPASLSVSVGETVTITCRTSENIYNNLAWYQQKQGKSPQLLVYA | 265 |
| | ATNLADGVPSRFSGSGSGTQYSLKINSLQSEDFGNYYCQHFWGTPLTFGA | |
| | GTKLELK | |

In some aspects, the anti-transferrin receptor antibody comprises a VH region and a VL region as illustrated in Table 5.

TABLE 5

| | 13E4_VH1 | 13E4_VH2 | 13E4_VH3 | 13E4_VH4 |
|---|---|---|---|---|
| | (SEQ ID NO: 256) | (SEQ ID NO: 257) | (SEQ ID NO: 258) | (SEQ ID NO: 259) |
| 13E4_VL1 | SEQ ID NO: 256 + | SEQ ID NO: 257 + | SEQ ID NO: 258 + | SEQ ID NO: 259 + |
| (SEQ ID NO: 261) | SEQ ID NO: 261 | SEQ ID NO: 261 | SEQ ID NO: 261 | SEQ ID NO: 261 |
| 13E4_VL2 | SEQ ID NO: 256 + | SEQ ID NO: 257 + | SEQ ID NO: 258 + | SEQ ID NO: 259 + |
| (SEQ ID NO: 262) | SEQ ID NO: 262 | SEQ ID NO: 262 | SEQ ID NO: 262 | SEQ ID NO: 262 |
| 13E4_VL3 | SEQ ID NO: 256 + | SEQ ID NO: 257 + | SEQ ID NO: 258 + | SEQ ID NO: 259 + |
| (SEQ ID NO: 263) | SEQ ID NO: 263 | SEQ ID NO: 263 | SEQ ID NO: 263 | SEQ ID NO: 263 |
| 13E4_VL4 | SEQ ID NO: 256 + | SEQ ID NO: 257 + | SEQ ID NO: 258 + | SEQ ID NO: 259 + |
| (SEQ ID NO: 264) | SEQ ID NO: 264 | SEQ ID NO: 264 | SEQ ID NO: 264 | SEQ ID NO: 264 |

Other available anti-TfR antibodies are described in: WO 2022/020105, WO 2021/154476, WO 2021/154477, WO2023/283620, US2023/0088865, U.S. Pat. Nos. 11,672,872, 11,839,660, 11,969,475, WO2024/006976, WO2025/085352, WO2024/036096, WO2024/026474, and US2025/235549, each of which is incorporated herein by reference in its entirety.

In some aspects, an anti-transferrin receptor antibody described herein comprises an IgG framework, an IgA framework, an IgE framework, or an IgM framework. In some instances, the anti-transferrin receptor antibody comprises an IgG framework (e.g., IgG1, IgG2, IgG3, or IgG4). In some cases, the anti-transferrin receptor antibody comprises an IgG1 framework. In some cases, the anti-transferrin receptor antibody comprises an IgG2 (e.g., an IgG2a or IgG2b) framework. In some cases, the anti-transferrin receptor antibody comprises an IgG2a framework. In some cases, the anti-transferrin receptor antibody comprises an IgG2b framework. In some cases, the anti-transferrin receptor antibody comprises an IgG3 framework. In some cases, the anti-transferrin receptor antibody comprises an IgG4 framework.

In some cases, an anti-transferrin receptor antibody comprises one or more mutations in a framework region, e.g., in the CHT domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional instances, the one or more mutations are to modulate glycosylation.

In some aspects, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some instances, the Fc region comprises a mutation at residue position L234, L235, D265, N297, K322, L328, or P329, or a combination thereof. In some instances, the mutations comprise L234 and L235 in combination with a mutation at residue position K322, L328, or P329. In some cases, the Fc region comprises mutations at L234, L235, and K322. In some cases, the Fc region comprises mutations at L234, L235, and L328. In some cases, the Fc region comprises mutations at L234, L235, and P329. In some cases, the Fc region comprises mutations at D265 and N297. In some cases, the residue position is in reference to IgG1.

In some instances, the Fc region comprises L234A, L235A, D265A, N297G, K322G, L328R, or P329G, or a combination thereof. In some instances, the Fc region comprises L234A and L235A in combination with K322G, L328R, or P329G. In some cases, the Fc region comprises L234A, L235A, and K322G. In some cases, the Fc region comprises L234A, L235A, and L328R. In some cases, the Fc region comprises L234A, L235A, and P329G. In some cases, the Fc region comprises D265A and N297G. In some cases, the residue position is in reference to IgG1.

In some instances, the Fc region comprises a mutation at residue position L235, L236, D265, N297, K322, L328, or P329, or a combination of the mutations. In some instances, the Fc region comprises mutations at L235 and L236. In some instances, the Fc region comprises mutations at L235 and L236 in combination with a mutation at residue position K322, L328, or P329. In some cases, the Fc region comprises mutations at L235, L236, and K322. In some cases, the Fc region comprises mutations at L235, L236, and L328. In some cases, the Fc region comprises mutations at L235, L236, and P329. In some cases, the Fc region comprises mutations at D265 and N297. In some cases, the residue position is in reference to IgG2b.

In some aspects, the Fc region comprises L235A, L236A, D265A, N297G, K322G, L328R, or P329G, or a combination thereof. In some instances, the Fc region comprises L235A and L236A. In some instances, the Fc region comprises L235A and L236A in combination with K322G, L328R, or P329G. In some cases, the Fc region comprises L235A, L236A, and K322G. In some cases, the Fc region comprises L235A, L236A, and L328R. In some cases, the Fc region comprises L235A, L236A, and P329G. In some cases, the Fc region comprises D265A and N297G. In some cases, the residue position is in reference to IgG2b.

In some aspects, the Fc region comprises a mutation at residue position L233, L234, D264, N296, K321, L327, or P328, wherein the residues correspond to positions 233, 234, 264, 296, 321, 327, and 328 of SEQ ID NO: 270. In some instances, the Fc region comprises mutations at L233 and L234. In some instances, the Fc region comprises mutations at L233 and L234 in combination with a mutation at residue position K321, L327, or P328. In some cases, the Fc region comprises mutations at L233, L234, and K321. In some cases, the Fc region comprises mutations at L233, L234, and L327. In some cases, the Fc region comprises mutations at L233, L234, and K321. In some cases, the Fc region comprises mutations at L233, L234, and P328. In some instances, the Fc region comprises mutations at D264 and N296. In some cases, equivalent positions to residue L233, L234, D264, N296, K321, L327, or P328 in an IgG1, IgG2, IgG3, or IgG4 framework are contemplated. In some cases, mutations to a residue that corresponds to residue L233, L234, D264, N296, K321, L327, or P328 of SEQ ID NO: 270 in an IgG1, IgG2, or IgG4 framework are also contemplated.

In some aspects, the Fc region comprises L233A, L234A, D264A, N296G, K321G, L327R, or P328G, wherein the residues correspond to positions 233, 234, 264, 296, 321, 327, and 328 of SEQ ID NO: 270. In some instances, the Fc region comprises L233A and L234A. In some instances, the Fc region comprises L233A and L234A in combination with K321G, L327R, or P328G. In some cases, the Fc region comprises L233A, L234A, and K321G. In some cases, the Fc region comprises L233A, L234A, and L327R. In some cases, the Fc region comprises L233A, L234A, and K321G. In some cases, the Fc region comprises L233A, L234A, and P328G. In some instances, the Fc region comprises D264A and N296G.

In some aspects, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some aspects, an anti-transferrin receptor antibody described herein is a full-length antibody, comprising a heavy chain (HC) and a light chain (LC). In some cases, the heavy chain (HC) comprises a sequence selected from Table 6. In some cases, the light chain (LC) comprises a sequence selected from Table 7. The underlined region denotes the respective CDRs.

TABLE 6

| NAME | HC SEQUENCE | SEQ ID NO: |
|---|---|---|
| 13E4_VH1 | QVQLVQSGAEVKKPGASVKVSCKASGYTFTNYWMHWVRQ | 270 |
| | APGQGLEWMGEINPINGRSNYAQKFQGRVTLTVDTSISTAY | |
| | MELSRLRSDDTAVYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH1_a | QVQLVQSGAEVKKPGASVKVSCKASGYTFTNYWMHWVRQ | 271 |
| | APGQGLEWMGEINPINGRSNYAQKFQGRVTLTVDTSISTAY | |
| | MELSRLRSDDTAVYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH1_b | QVQLVQSGAEVKKPGASVKVSCKASGYTFTNYWMHWVRQ | 272 |
| | APGQGLEWMGEINPINGRSNYAQKFQGRVTLTVDTSISTAY | |
| | MELSRLRSDDTAVYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCGVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH1_c | QVQLVQSGAEVKKPGASVKVSCKASGYTFTNYWMHWVRQ | 273 |
| | APGQGLEWMGEINPINGRSNYAQKFQGRVTLTVDTSISTAY | |
| | MELSRLRSDDTAVYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAR | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH1_d | QVQLVQSGAEVKKPGASVKVSCKASGYTFTNYWMHWVRQ | 274 |
| | APGQGLEWMGEINPINGRSNYAQKFQGRVTLTVDTSISTAY | |
| | MELSRLRSDDTAVYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | GAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH1_e | QVQLVQSGAEVKKPGASVKVSCKASGYTFTNYWMHWVRQ | 275 |
| | APGQGLEWMGEINPINGRSNYAQKFQGRVTLTVDTSISTAY | |
| | MELSRLRSDDTAVYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPELLGGPSVFLFPPKPK | |
| | DTLMISRTPEVTCVVVAVSHEDPEVKFNWYVDGVEVHNAK | |
| | TKPREEQYGSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH2 | QVQLVQSGAEVKKPGASVKVSCKASGYTFTNYWMHWVRQ | 276 |
| | APGQGLEWIGEINPINGRSNYAEKFQGRVTLTVDTSSSTAYM | |
| | ELSRLRSDDTAVYYCARGTRAMHYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKRVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSREEMTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPG | |
| 13E4_VH2_a | QVQLVQSGAEVKKPGASVKVSCKASGYTFTNYWMHWVRQ | 277 |
| | APGQGLEWIGEINPINGRSNYAEKFQGRVTLTVDTSSSTAYM | |
| | ELSRLRSDDTAVYYCARGTRAMHYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKRVEPKSCDKTHTCPPCPAPEAAGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSREEMTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPG | |
| 13E4_VH2_b | QVQLVQSGAEVKKPGASVKVSCKASGYTFTNYWMHWVRQ | 278 |
| | APGQGLEWIGEINPINGRSNYAEKFQGRVTLTVDTSSSTAYM | |
| | ELSRLRSDDTAVYYCARGTRAMHYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKRVEPKSCDKTHTCPPCPAPEAAGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCGVSNKALP | |
| | APIEKTISKAKGQPREPQVYTLPPSREEMTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPG | |
| 13E4_VH2 c | QVQLVQSGAEVKKPGASVKVSCKASGYTFTNYWMHWVRQ | 279 |
| | APGQGLEWIGEINPINGRSNYAEKFQGRVTLTVDTSSSTAYM | |
| | ELSRLRSDDTAVYYCARGTRAMHYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKRVEPKSCDKTHTCPPCPAPEAAGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKARP | |
| | APIEKTISKAKGQPREPQVYTLPPSREEMTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPG | |
| 13E4_VH2_d | QVQLVQSGAEVKKPGASVKVSCKASGYTFTNYWMHWVRQ | 280 |
| | APGQGLEWIGEINPINGRSNYAEKFQGRVTLTVDTSSSTAYM | |
| | ELSRLRSDDTAVYYCARGTRAMHYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKRVEPKSCDKTHTCPPCPAPEAAGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALG | |
| | APIEKTISKAKGQPREPQVYTLPPSREEMTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPG | |
| 13E4_VH2_e | QVQLVQSGAEVKKPGASVKVSCKASGYTFTNYWMHWVRQ | 281 |
| | APGQGLEWIGEINPINGRSNYAEKFQGRVTLTVDTSSSTAYM | |
| | ELSRLRSDDTAVYYCARGTRAMHYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKRVEPKSCDKTHTCPPCPAPELLGGPSVFLFPPKPKD | |
| | TLMISRTPEVTCVVVAVSHEDPEVKFNWYVDGVEVHNAKT | |
| | KPREEQYGSTYRVVSVLTVLHQDWLNGKEYKCKVSNKALP | |
| | APIEKTISKAKGQPREPQVYTLPPSREEMTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPG | |
| 13E4_VH3 | QVQLVQSGAEVKKPGASVKVSCKASGYTFTNYWMHWVRQ | 282 |
| | APGQGLEWMGEINPIQGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH3_a | QVQLVQSGAEVKKPGASVKVSCKASGYTFTNYWMHWVRQ | 283 |
| | APGQGLEWMGEINPIQGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH3_b | QVQLVQSGAEVKKPGASVKVSCKASGYTFTNYWMHWVRQ | 284 |
| | APGQGLEWMGEINPIQGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCGVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH3_c | QVQLVQSGAEVKKPGASVKVSCKASGYTFTNYWMHWVRQ | 285 |
| | APGQGLEWMGEINPIQGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAR | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH3_d | QVQLVQSGAEVKKPGASVKVSCKASGYTFTNYWMHWVRQ | 286 |
| | APGQGLEWMGEINPIQGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | GAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH3_e | QVQLVQSGAEVKKPGASVKVSCKASGYTFTNYWMHWVRQ | 287 |
| | APGQGLEWMGEINPIQGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPELLGGPSVFLFPPKPK | |
| | DTLMISRTPEVTCVVVAVSHEDPEVKFNWYVDGVEVHNAK | |
| | TKPREEQYGSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH4 | QVQLVQSGAEVKKPGASVKVSCKASGYTFTNYWMHWVRQ | 288 |
| | APGQGLEWMGEINPINGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH4_a | QVQLVQSGAEVKKPGASVKVSCKASGYTFTNYWMHWVRQ | 289 |
| | APGQGLEWMGEINPINGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH4_b | QVQLVQSGAEVKKPGASVKVSCKASGYTFTNYWMHWVRQ | 290 |
| | APGQGLEWMGEINPINGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCGVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH4_c | QVQLVQSGAEVKKPGASVKVSCKASGYTFTNYWMHWVRQ | 291 |
| | APGQGLEWMGEINPINGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAR | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH4_d | QVQLVQSGAEVKKPGASVKVSCKASGYTFTNYWMHWVRQ | 292 |
| | APGQGLEWMGEINPINGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | GAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 13E4_VH4_e | QVQLVQSGAEVKKPGASVKVSCKASGYTFTNYWMHWVRQ | 293 |
| | APGQGLEWMGEINPINGRSNYAEKFQGRVTLTVDTSSSTAY | |
| | MELSSLRSEDTATYYCARGTRAMHYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDKTHTCPPCPAPELLGGPSVFLFPPKPK | |
| | DTLMISRTPEVTCVVVAVSHEDPEVKFNWYVDGVEVHNAK | |
| | TKPREEQYGSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |

TABLE 7

| NAME | LC SEQUENCE | SEQ ID NO: |
|---|---|---|
| 13E4_VL1 | DIQMTQSPSSLSASVGDRVTITCRTSENIYNNLAWYQQKPGKS | 294 |
| | PKLLIYAATNLADGVPSRFSGSGSGTDYTLTISSLQPEDFATYY | |
| | CQHFWGTPLTFGGGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| 13E4_VL2 | DIQMTQSPSSLSASVGDRVTITCRTSENIYNNLAWYQQKPGK | 295 |
| | APKLLIYAATNLADGVPSRFSGSGSGTDYTLTISSLQPEDFATY | |
| | YCQHFWGTPLTFGGGTKVEIKRTVAAPSVFIFPPSDEQLKSGT | |
| | ASVVCLLNNFYPREAKVQWKVDNALQSGNSQESVTEQDSKD | |
| | STYSLSSTLTLSKADYEKHKVYACEVTHQGLSSPVTKSFNRGEC | |
| 13E4_VL3 | DIQMTQSPSSLSASVGDRVTITCRTSENIYNNLAWYQQKPGK | 296 |
| | APKLLIYAATNLAEGVPSRFSGSGSGTDYTLTISSLQPEDFATY | |
| | YCQHFWGTPLTFGGGTKVEIKRTVAAPSVFIFPPSDEQLKSGT | |
| | ASVVCLLNNFYPREAKVQWKVDNALQSGNSQESVTEQDSKD | |
| | STYSLSSTLTLSKADYEKHKVYACEVTHQGLSSPVTKSFNRGEC | |
| 13E4_VL4 | DIQMTQSPSSLSASVGDRVTITCRTSENIYSNLAWYQQKPGKA | 297 |
| | PKLLIYAGTNLADGVPSRFSGSGSGTDYTLTISSLQPEDFANY | |
| | YCQHFWGTPLTFGGGTKVEIKRTVAAPSVFIFPPSDEQLKSGT | |
| | ASVVCLLNNFYPREAKVQWKVDNALQSGNSQESVTEQDSKD | |
| | STYSLSSTLTLSKADYEKHKVYACEVTHQGLSSPVTKSFNRGEC | |

In some aspects, an anti-transferrin receptor antibody described herein has an improved serum half-life compared to a reference anti-transferrin receptor antibody. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference anti-transferrin receptor antibody.

In some aspects, the binding moiety is conjugated to a polynucleic acid molecule non-specifically. In some instances, the binding moiety is conjugated to a polynucleic acid molecule via a lysine residue or a cysteine residue, in a non-site specific manner. In some instances, the binding moiety is conjugated to a polynucleic acid molecule via a lysine residue (e.g., lysine residue present in the binding moiety) in a non-site specific manner. In some cases, the binding moiety is conjugated to a polynucleic acid molecule via a cysteine residue (e.g., cysteine residue present in the binding moiety) in a non-site specific manner.

In some aspects, the binding moiety is conjugated to a polynucleic acid molecule in a site-specific manner. In some instances, the binding moiety is conjugated to a polynucleic acid molecule through a lysine residue, a cysteine residue, at the 5′-terminus, at the 3′-terminus, an unnatural amino acid, or an enzyme-modified or enzyme-catalyzed residue, via a site-specific manner. In some instances, the binding moiety is conjugated to a polynucleic acid molecule through a lysine residue (e.g., lysine residue present in the binding moiety) via a site-specific manner. In some instances, the binding moiety is conjugated to a polynucleic acid molecule through a cysteine residue (e.g., cysteine residue present in the binding moiety) via a site-specific manner. In some instances, the binding moiety is conjugated to a polynucleic acid molecule at the 5′-terminus via a site-specific manner. In some instances, the binding moiety is conjugated to a polynucleic acid molecule at the 3′-terminus via a site-specific manner. In some instances, the binding moiety is conjugated to a polynucleic acid molecule through an unnatural amino acid via a site-specific manner. In some instances, the binding moiety is conjugated to a polynucleic acid molecule through an enzyme-modified or enzyme-catalyzed residue via a site-specific manner.

In some aspects, one or more polynucleic acid molecule is conjugated to a binding moiety. In some instances, about 1, 2, 3, 4, 5, 6, 7, 8, 9, 10, 11, 12, 13, 14, 15, 16, or more polynucleic acid molecules are conjugated to one binding moiety. In some instances, about 1 polynucleic acid molecule is conjugated to one binding moiety. In some instances, about 2 polynucleic acid molecules are conjugated to one binding moiety. In some instances, about 3 polynucleic acid molecules are conjugated to one binding moiety. In some instances, about 4 polynucleic acid molecules are conjugated to one binding moiety. In some instances, about 5 polynucleic acid molecules are conjugated to one binding moiety. In some instances, about 6 polynucleic acid molecules are conjugated to one binding moiety. In some instances, about 7 polynucleic acid molecules are conjugated to one binding moiety. In some instances, about 8 polynucleic acid molecules are conjugated to one binding moiety. In some instances, about 9 polynucleic acid molecules are conjugated to one binding moiety. In some instances, about 10 polynucleic acid molecules are conjugated to one binding moiety. In some instances, about 11 polynucleic acid molecules are conjugated to one binding moiety. In some instances, about 12 polynucleic acid molecules are conjugated to one binding moiety. In some instances, about 13 polynucleic acid molecules are conjugated to one binding moiety. In some instances, about 14 polynucleic acid molecules are conjugated to one binding moiety. In some instances, about 15 polynucleic acid molecules are conjugated to one binding moiety. In some instances, about 16 polynucleic acid molecules are conjugated to one binding moiety. In some cases, the one or more polynucleic acid molecules are the same. In other cases, the one or more polynucleic acid molecules are different.

In some aspects, the number of polynucleic acid molecule conjugated to a binding moiety forms a ratio. In some instances, the ratio is referred to as a DAR (drug-to-antibody ratio), in which the drug as referred to herein is the polynucleic acid molecule. In some instances, the DAR of the polynucleic acid molecule to binding moiety is about 1, 2, 3, 4, 5, 6, 7, 8, 9, 10, 11, 12, 13, 14, 15, 16, or greater. In some instances, the DAR of the polynucleic acid molecule to binding moiety is about 1 or greater. In some instances, the DAR of the polynucleic acid molecule to binding moiety is about 2 or greater. In some instances, the DAR of the polynucleic acid molecule to binding moiety is about 3 or greater. In some instances, the DAR of the polynucleic acid molecule to binding moiety is about 4 or greater. In some instances, the DAR of the polynucleic acid molecule to binding moiety is about 5 or greater. In some instances, the DAR of the polynucleic acid molecule to binding moiety is about 6 or greater. In some instances, the DAR of the polynucleic acid molecule to binding moiety is about 7 or greater. In some instances, the DAR of the polynucleic acid molecule to binding moiety is about 8 or greater. In some instances, the DAR of the polynucleic acid molecule to binding moiety is about 9 or greater. In some instances, the DAR of the polynucleic acid molecule to binding moiety is about 10 or greater. In some instances, the DAR of the polynucleic acid molecule to binding moiety is about 11 or greater. In some instances, the DAR of the polynucleic acid molecule to binding moiety is about 12 or greater.

In some instances, the DAR of the polynucleic acid molecule to binding moiety is about 1, 2, 3, 4, 5, 6, 7, 8, 9, 10, 11, 12, 13, 14, 15, or 16. In some instances, the DAR of the polynucleic acid molecule to binding moiety is about 1. In some instances, the DAR of the polynucleic acid molecule to binding moiety is about 2. In some instances, the DAR of the polynucleic acid molecule to binding moiety is about 3. In some instances, the DAR of the polynucleic acid molecule to binding moiety is about 4. In some instances, the DAR of the polynucleic acid molecule to binding moiety is about 5. In some instances, the DAR of the polynucleic acid molecule to binding moiety is about 6. In some instances, the DAR of the polynucleic acid molecule to binding moiety is about 7. In some instances, the DAR of the polynucleic acid molecule to binding moiety is about 8. In some instances, the DAR of the polynucleic acid molecule to binding moiety is about 9. In some instances, the DAR of the polynucleic acid molecule to binding moiety is about 10. In some instances, the DAR of the polynucleic acid molecule to binding moiety is about 11. In some instances, the DAR of the polynucleic acid molecule to binding moiety is about 12. In some instances, the DAR of the polynucleic acid molecule to binding moiety is about 13. In some instances, the DAR of the polynucleic acid molecule to binding moiety is about 14. In some instances, the DAR of the polynucleic acid molecule to binding moiety is about 15. In some instances, the DAR of the polynucleic acid molecule to binding moiety is about 16.

In some instances, the DAR of the polynucleic acid molecule to binding moiety is 1, 2, 3, 4, 5, 6, 7, 8, 9, 10, 11, 12, 13, 14, 15, or 16. In some instances, the DAR of the polynucleic acid molecule to binding moiety is 1. In some instances, the DAR of the polynucleic acid molecule to binding moiety is 2. In some instances, the DAR of the polynucleic acid molecule to binding moiety is 4. In some instances, the DAR of the polynucleic acid molecule to binding moiety is 6. In some instances, the DAR of the polynucleic acid molecule to binding moiety is 8. In some instances, the DAR of the polynucleic acid molecule to binding moiety is 12.

In some aspects, the pharmaceutical composition comprises a plurality of antibody-polynucleic acid conjugates. In some instances, the plurality of antibody-polynucleic acid conjugates in the composition has different DARs. In some instances, at least two of the antibody-polynucleic acid conjugates in the composition have different DARs to each other. In some instances, the DAR is an average DAR (drug-to-antibody ratio), which is an average number of the DARs of the plurality of antibody-polynucleic acid conjugates in the composition. In some instances, the average DAR of the polynucleic acid molecule to antibody is about 1, 2, 3, 4, 5, 6, 7, 8, 9, 10, 11, 12, 13, 14, 15, 16, or greater. In some instances, the average DAR of the polynucleic acid molecule to antibody is approximately 1, 2, 3, 4, 5, 6, 7, 8, 9, 10, 11, 12, 13, 14, 15, 16, or greater. In some instances, the average DAR includes whole number as well as fractions or decimal of a DAR. In some instances, the average DAR of the polynucleic acid molecule to antibody is about 1 or greater. In some instances, the average DAR of the polynucleic acid molecule to antibody is about 2 or greater. In some instances, the average DAR of the polynucleic acid molecule to antibody is about 3 or greater. In some instances, the average DAR of the polynucleic acid molecule to antibody is about 4 or greater. In some instances, the average DAR of the polynucleic acid molecule to antibody is about 5 or greater. In some instances, the average DAR of the polynucleic acid molecule to antibody is about 6 or greater. In some instances, the average DAR of the polynucleic acid molecule to antibody is about 7 or greater. In some instances, the average DAR of the polynucleic acid molecule to antibody is about 8 or greater. In some instances, the average DAR of the polynucleic acid molecule to antibody is about 9 or greater. In some instances, the average DAR of the polynucleic acid molecule to antibody is about 10 or greater. In some instances, the average DAR of the polynucleic acid molecule to antibody is about 11 or greater. In some instances, the average DAR of the polynucleic acid molecule to antibody is about 12 or greater.

In some instances, the average DAR of the polynucleic acid molecule to antibody is about 1, 2, 3, 4, 5, 6, 7, 8, 9, 10, 11, 12, 13, 14, 15, or 16. In some instances, the average DAR of the polynucleic acid molecule to antibody is about 1. In some instances, the average DAR of the polynucleic acid molecule to antibody is about 2. In some instances, the average DAR of the polynucleic acid molecule to antibody is about 3. In some instances, the average DAR of the polynucleic acid molecule to antibody is about 4. In some instances, the average DAR of the polynucleic acid molecule to antibody is about 5. In some instances, the average DAR of the polynucleic acid molecule to antibody is about 6. In some instances, the average DAR of the polynucleic acid molecule to antibody is about 7. In some instances, the average DAR of the polynucleic acid molecule to antibody is about 7.2. In some instances, the average DAR of the polynucleic acid molecule to antibody is about 7.4. In some instances, the average DAR of the polynucleic acid molecule to antibody is about 7.6. In some instances, the average DAR of the polynucleic acid molecule to antibody is about 7.8. In some instances, the average DAR of the polynucleic acid molecule to antibody is about 8. In some instances, the average DAR of the polynucleic acid molecule to antibody is about 9. In some instances, the average DAR of the polynucleic acid molecule to antibody is about 10. In some instances, the average DAR of the polynucleic acid molecule to antibody is about 11. In some instances, the average DAR of the polynucleic acid molecule to antibody is about 12. In some instances, the average DAR of the polynucleic acid molecule to antibody is about 13. In some instances, the average DAR of the polynucleic acid molecule to antibody is about 14. In some instances, the DAR of the polynucleic acid molecule to antibody is about 15. In some instances, the average DAR of the polynucleic acid molecule to antibody is about 16.

In some instances, the average DAR of the polynucleic acid molecule to antibody is 1, 2, 3, 4, 5, 6, 7, 8, 9, 10, 11, 12, 13, 14, 15, or 16. In some instances, the average DAR of the polynucleic acid molecule to antibody is 1. In some instances, the average DAR of the polynucleic acid molecule to antibody is 2. In some instances, the average DAR of the polynucleic acid molecule to antibody is 4. In some instances, the average DAR of the polynucleic acid molecule to antibody is 6. In some instances, the average DAR of the polynucleic acid molecule to antibody is 8. In some instances, the average DAR of the polynucleic acid molecule to antibody is 12. In some instances, the average DAR of the polynucleic acid molecule to antibody is 16.

In some instances, the average DAR of the polynucleic acid molecule to antibody is in the range of 1.5-2.5, 2.5-3.5, 3.5-4.5, 4.5-5.5, 5.5-6.5, 6.5-7.5, 7.5-8.5, 8.5-9.5, 9.5-10.5, 10.5-11.5, 11.5-12.5, 12.5-13.5, 13.5-14.5, 14.5-15.5, 15.5-16.5, or 16.5-17.5. In some instances, the average DAR of the polynucleic acid molecule to antibody is in the range of 1.5-2.5. In some instances, the average DAR of the polynucleic acid molecule to antibody is in the range of 2.5-3.5. In some instances, the average DAR of the polynucleic acid molecule to antibody is in the range of 3.5-4.5. In some instances, the average DAR of the polynucleic acid molecule to antibody is in the range of 4.5-5.5. In some instances, the average DAR of the polynucleic acid molecule to antibody is in the range of 5.5-6.5. In some instances, the average DAR of the polynucleic acid molecule to antibody is in the range of 6.5-7.5. In some instances, the average DAR of the polynucleic acid molecule to antibody is in the range of 7.0-7.5. In some instances, the average DAR of the polynucleic acid molecule to antibody is in the range of 7.5-8.5. In some instances, the average DAR of the polynucleic acid molecule to antibody is in the range of 8.5-9.5. In some instances, the average DAR of the polynucleic acid molecule to antibody is in the range of 9.5-10.5. In some instances, the average DAR of the polynucleic acid molecule to antibody is in the range of 10.5-11.5. In some instances, the average DAR of the polynucleic acid molecule to antibody is in the range of 11.5-12.5. In some instances, the average DAR of the polynucleic acid molecule to antibody is in the range of 12.5-13.5. In some instances, the average DAR of the polynucleic acid molecule to antibody is in the range of 13.5-14.5. In some instances, the average DAR of the polynucleic acid molecule to antibody is in the range of 14.5-15.5. In some instances, the DAR of the polynucleic acid molecule to antibody is in the range of 15.5-16.5. In some instances, the average DAR of the polynucleic acid molecule to antibody is in the range of 16.5-17.5.

In some instances, a conjugate comprising polynucleic acid molecule and binding moiety has improved activity as compared to a conjugate comprising polynucleic acid molecule without a binding moiety. In some instances, improved activity results in enhanced biologically relevant functions, e.g., improved stability, affinity, binding, functional activity, and efficacy in treatment or prevention of a disease state. In some instances, the disease state is a result of one or more mutated exons of a gene. In some instances, the conjugate comprising polynucleic acid molecule and binding moiety results in increased exon skipping of the one or more mutated exons as compared to the conjugate comprising polynucleic acid molecule without a binding moiety. In some instances, exon skipping is increased by at least or about 5%, 10%, 20%, 25%, 30%, 40%, 50%, 60%, 70%, 80%, 90%, 95%, or more than 95% in the conjugate comprising polynucleic acid molecule and binding moiety as compared to the conjugate comprising polynucleic acid molecule without a binding moiety.

In some aspects, an antibody or antigen binding fragment is further modified using conventional techniques known in the art, for example, by using amino acid deletion, insertion, substitution, addition, and/or by recombination and/or any other modification (e.g., posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor. Methods for introducing such modifications in the nucleic acid sequence underlying the amino acid sequence of an antibody or antigen binding fragment is well known to the person skilled in the art.

In some instances, an antigen binding fragment further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such embodiments, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some aspects, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to that the antibody or its antigen binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further embodiments, the binding is exemplified by the specificity of a “key-lock-principle”. Thus, in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or antigen binding fragment or a reduced off-target effect. For example, the antibody or antigen binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Additional Binding Moieties

In some aspects, the binding moiety is a plasma protein. In some instances, the plasma protein comprises albumin. In some instances, the binding moiety is albumin. In some instances, albumin is conjugated by one or more of a conjugation chemistry described herein to a polynucleic acid molecule. In some instances, albumin is conjugated by native ligation chemistry to a polynucleic acid molecule. In some instances, albumin is conjugated by lysine conjugation to a polynucleic acid molecule.

In some instances, the binding moiety is a steroid. Exemplary steroids include cholesterol, phospholipids, di- and triacylglycerols, fatty acids, hydrocarbons that are saturated, unsaturated, comprise substitutions, or combinations thereof. In some instances, the steroid is cholesterol. In some instances, the binding moiety is cholesterol. In some instances, cholesterol is conjugated by one or more of a conjugation chemistry described herein to a polynucleic acid molecule. In some instances, cholesterol is conjugated by native ligation chemistry to a polynucleic acid molecule. In some instances, cholesterol is conjugated by lysine conjugation to a polynucleic acid molecule.

In some instances, the binding moiety is a polymer, including but not limited to polynucleic acid molecule aptamers that bind to specific surface markers on cells. In this instance the binding moiety is a polynucleic acid that does not hybridize to a target gene or mRNA, but instead is capable of selectively binding to a cell surface marker similarly to an antibody binding to its specific epitope of a cell surface marker.

In additional cases, the binding moiety is a small molecule. In some instances, the small molecule is an antibody-recruiting small molecule. In some cases, the antibody-recruiting small molecule comprises a target-binding terminus and an antibody-binding terminus, in which the target-binding terminus is capable of recognizing and interacting with a cell surface receptor. For example, in some instances, the target-binding terminus comprising a glutamate urea compound enables interaction with PSMA, thereby, enhances an antibody interaction with a cell that expresses PSMA. In some instances, a binding moiety is a small molecule described in Zhang et al., “A remote arene-binding site on prostate specific membrane antigen revealed by antibody-recruiting small molecules,” J Am Chem Soc. 132(36): 12711-12716 (2010); or McEnaney, et al., “Antibody-recruiting molecules: an emerging paradigm for engaging immune function in treating human disease,” ACS Chem Biol. 7(7): 1139-1151 (2012).

### Production of Antibodies or Antigen Binding Fragment Thereof

In some aspects, techniques developed for the production of “chimeric antibodies” (Morrison et al., 1984, Proc. Natd. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

### Conjugation Chemistry

In some aspects, a polynucleic acid molecule is conjugated to a binding moiety. In some aspects, a polynucleic acid molecule is conjugated to a binding moiety via a linker. In some instances, the binding moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of binding moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the binding moiety is an antibody or antigen binding fragment thereof. In some instances, the polynucleic acid molecule is further conjugated to a polymer, and optionally an endosomolytic moiety.

In some aspects, the polynucleic acid molecule is conjugated to the binding moiety by a chemical ligation process. In some instances, the polynucleic acid molecule is conjugated to the binding moiety by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J Am. Chem. Soc. 1997, 119, 4325-4329; Hackeng, et al. “Protein synthesis by native chemical ligation: Expanded scope by using straightforward methodology.,” Proc. Natd. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some aspects, the polynucleic acid molecule is conjugated to the binding moiety either site-specifically or non-specifically via native ligation chemistry.

In some instances, the polynucleic acid molecule is conjugated to the binding moiety by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the polynucleic acid molecule is conjugated to the binding moiety by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the polynucleic acid molecule is conjugated to the binding moiety by a site-directed method utilizing an enzyme-catalyzed process. In some instances, the site-directed method utilizes SMARTag™ technology (Catalent, Inc.). In some instances, the SMARTag™ technology comprises generation of a formylglycine (FG1y) residue from cysteine by formylglycine-generating enzyme (FGE) through an oxidation process under the presence of an aldehyde tag and the subsequent conjugation of FG1y to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the polynucleic acid molecule is conjugated to the binding moiety utilizing a microbial transglutaminase-catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some instances, the polynucleic acid molecule is conjugated to the binding moiety by a method as described in PCT Publication No. WO2014/140317, which utilizes a sequence-specific transpeptidase.

In some instances, the polynucleic acid molecule is conjugated to the binding moiety by a method as described in U.S. Patent Publication Nos. 2015/0105539 and 2015/0105540.

In some instances, the polynucleic acid molecule is conjugated to a thiol reactive group on the binding moiety (e.g., traut's alkyl thiols, substitute alkyl thiols). In some instances, the polynucleic acid molecule is conjugated to the binding moiety via a disulfide linkage to a pyridyl (pyr) moiety. In some instances, the polynucleic acid molecule is conjugated to the binding moiety by squaric acid ester-based synthesis.

### Polymer Conjugating Moiety

In some aspects, a polymer moiety is further conjugated to a polynucleic acid molecule described herein, a binding moiety described herein, or in combinations thereof. In some instances, a polymer moiety is conjugated a polynucleic acid molecule via a linker. In some instances, a binding moiety is conjugated to a polynucleic acid molecule via a first linker. In some instances, a polymer moiety is conjugated to a polynucleic acid molecule via a second linker. In some cases, the first linker and the second linker are the same. In some cases, the first linker and the second linker are different. In some cases, a polymer moiety is conjugated to a binding moiety. In other cases, a polymer moiety is conjugated to a polynucleic acid molecule-binding moiety molecule. In additional cases, a polymer moiety is conjugated, as illustrated supra.

In some instances, the polymer moiety is a natural or synthetic polymer, consisting of long chains of branched or unbranched monomers, and/or cross-linked network of monomers in two or three dimensions. In some instances, the polymer moiety includes a polysaccharide, lignin, rubber, or polyalkylen oxide (e.g., polyethylene glycol). In some instances, the at least one polymer moiety includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylene terephthalate (also known as poly(ethylene terephthalate), PET, PETG, or PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the polymer moiety comprises polyalkylene oxide. In some instances, the polymer moiety comprises PEG. In some instances, the polymer moiety comprises polyethylene imide (PEI) or hydroxy ethyl starch (HES).

In some instances, the polymer moiety is a PEG moiety. In some instances, the PEG moiety is conjugated at the 5′ terminus of the polynucleic acid molecule while the binding moiety is conjugated at the 3′ terminus of the polynucleic acid molecule. In some instances, the PEG moiety is conjugated at the 3′ terminus of the polynucleic acid molecule while the binding moiety is conjugated at the 5′ terminus of the polynucleic acid molecule. In some instances, the PEG moiety is conjugated to an internal site of the polynucleic acid molecule. In some instances, the PEG moiety, the binding moiety, or a combination thereof, are conjugated to an internal site of the polynucleic acid molecule. In some instances, the conjugation is a direct conjugation. In some instances, the conjugation is via native ligation.

In some aspects, the polyalkylene oxide (e.g., PEG) is a polydisperse or monodisperse compound. In some instances, polydisperse material comprises disperse distribution of different molecular weight of the material, characterized by mean weight (weight average) size and dispersity. In some instances, the monodisperse PEG comprises one size of molecules. In some aspects, the polymer moiety is poly- or monodispersed polyalkylene oxide (e.g., PEG) and the indicated molecular weight represents an average of the molecular weight of the polyalkylene oxide, e.g., PEG, molecules.

In some aspects, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some aspects, the polymer moiety is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some aspects, the polymer moiety is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some instances, the molecular weight of the polymer moiety is about 200 Da. In some instances, the molecular weight of the polymer moiety is about 300 Da. In some instances, the molecular weight of the polymer moiety is about 400 Da. In some instances, the molecular weight of the polymer moiety is about 500 Da. In some instances, the molecular weight of the polymer moiety is about 600 Da. In some instances, the molecular weight of the polymer moiety is about 700 Da. In some instances, the molecular weight of the polymer moiety is about 800 Da. In some instances, the molecular weight of the polymer moiety is about 900 Da. In some instances, the molecular weight of the polymer moiety is about 1000 Da. In some instances, the molecular weight of the polymer moiety is about 1100 Da. In some instances, the molecular weight of the polymer moiety is about 1200 Da. In some instances, the molecular weight of the polymer moiety is about 1300 Da. In some instances, the molecular weight of the polymer moiety is about 1400 Da. In some instances, the molecular weight of the polymer moiety is about 1450 Da. In some instances, the molecular weight of the polymer moiety is about 1500 Da. In some instances, the molecular weight of the polymer moiety is about 1600 Da. In some instances, the molecular weight of the polymer moiety is about 1700 Da. In some instances, the molecular weight of the polymer moiety is about 1800 Da. In some instances, the molecular weight of the polymer moiety is about 1900 Da. In some instances, the molecular weight of the polymer moiety is about 2000 Da. In some instances, the molecular weight of the polymer moiety is about 2100 Da. In some instances, the molecular weight of the polymer moiety is about 2200 Da. In some instances, the molecular weight of the polymer moiety is about 2300 Da. In some instances, the molecular weight of the polymer moiety is about 2400 Da. In some instances, the molecular weight of the polymer moiety is about 2500 Da. In some instances, the molecular weight of the polymer moiety is about 2600 Da. In some instances, the molecular weight of the polymer moiety is about 2700 Da. In some instances, the molecular weight of the polymer moiety is about 2800 Da. In some instances, the molecular weight of the polymer moiety is about 2900 Da. In some instances, the molecular weight of the polymer moiety is about 3000 Da. In some instances, the molecular weight of the polymer moiety is about 3250 Da. In some instances, the molecular weight of the polymer moiety is about 3350 Da. In some instances, the molecular weight of the polymer moiety is about 3500 Da. In some instances, the molecular weight of the polymer moiety is about 3750 Da. In some instances, the molecular weight of the polymer moiety is about 4000 Da. In some instances, the molecular weight of the polymer moiety is about 4250 Da. In some instances, the molecular weight of the polymer moiety is about 4500 Da. In some instances, the molecular weight of the polymer moiety is about 4600 Da. In some instances, the molecular weight of the polymer moiety is about 4750 Da. In some instances, the molecular weight of the polymer moiety is about 5000 Da. In some instances, the molecular weight of the polymer moiety is about 5500 Da. In some instances, the molecular weight of the polymer moiety is about 6000 Da. In some instances, the molecular weight of the polymer moiety is about 6500 Da. In some instances, the molecular weight of the polymer moiety is about 7000 Da. In some instances, the molecular weight of the polymer moiety is about 7500 Da. In some instances, the molecular weight of the polymer moiety is about 8000 Da. In some instances, the molecular weight of the polymer moiety is about 10,000 Da. In some instances, the molecular weight of the polymer moiety is about 12,000 Da. In some instances, the molecular weight of the polymer moiety is about 20,000 Da. In some instances, the molecular weight of the polymer moiety is about 35,000 Da. In some instances, the molecular weight of the polymer moiety is about 40,000 Da. In some instances, the molecular weight of the polymer moiety is about 50,000 Da. In some instances, the molecular weight of the polymer moiety is about 60,000 Da. In some instances, the molecular weight of the polymer moiety is about 100,000 Da.

In some aspects, the polyalkylene oxide (e.g., PEG) comprises discrete ethylene oxide units (e.g., four to about 48 ethylene oxide units). In some instances, the polyalkylene oxide comprising the discrete ethylene oxide units is a linear chain. In other cases, the polyalkylene oxide comprising the discrete ethylene oxide units is a branched chain.

In some instances, the polymer moiety is a polyalkylene oxide (e.g., PEG) comprising discrete ethylene oxide units. In some cases, the polymer moiety comprises between about 4 and about 48 ethylene oxide units. In some cases, the polymer moiety comprises about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, or about 48 ethylene oxide units.

In some instances, the polymer moiety is a discrete PEG comprising, e.g., between about 4 and about 48 ethylene oxide units. In some cases, the polymer moiety is a discrete PEG comprising, e.g., about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, or about 48 ethylene oxide units. In some cases, the polymer moiety is a discrete PEG comprising, e.g., about 4 ethylene oxide units. In some cases, the polymer moiety is a discrete PEG comprising, e.g., about 5 ethylene oxide units. In some cases, the polymer moiety is a discrete PEG comprising, e.g., about 6 ethylene oxide units. In some cases, the polymer moiety is a discrete PEG comprising, e.g., about 7 ethylene oxide units. In some cases, the polymer moiety is a discrete PEG comprising, e.g., about 8 ethylene oxide units. In some cases, the polymer moiety is a discrete PEG comprising, e.g., about 9 ethylene oxide units. In some cases, the polymer moiety is a discrete PEG comprising, e.g., about 10 ethylene oxide units. In some cases, the polymer moiety is a discrete PEG comprising, e.g., about 11 ethylene oxide units. In some cases, the polymer moiety is a discrete PEG comprising, e.g., about 12 ethylene oxide units. In some cases, the polymer moiety is a discrete PEG comprising, e.g., about 13 ethylene oxide units. In some cases, the polymer moiety is a discrete PEG comprising, e.g., about 14 ethylene oxide units. In some cases, the polymer moiety is a discrete PEG comprising, e.g., about 15 ethylene oxide units. In some cases, the polymer moiety is a discrete PEG comprising, e.g., about 16 ethylene oxide units. In some cases, the polymer moiety is a discrete PEG comprising, e.g., about 17 ethylene oxide units. In some cases, the polymer moiety is a discrete PEG comprising, e.g., about 18 ethylene oxide units. In some cases, the polymer moiety is a discrete PEG comprising, e.g., about 19 ethylene oxide units. In some cases, the polymer moiety is a discrete PEG comprising, e.g., about 20 ethylene oxide units. In some cases, the polymer moiety is a discrete PEG comprising, e.g., about 21 ethylene oxide units. In some cases, the polymer moiety is a discrete PEG comprising, e.g., about 22 ethylene oxide units. In some cases, the polymer moiety is a discrete PEG comprising, e.g., about 23 ethylene oxide units. In some cases, the polymer moiety is a discrete PEG comprising, e.g., about 24 ethylene oxide units. In some cases, the polymer moiety is a discrete PEG comprising, e.g., about 25 ethylene oxide units. In some cases, the polymer moiety is a discrete PEG comprising, e.g., about 26 ethylene oxide units. In some cases, the polymer moiety is a discrete PEG comprising, e.g., about 27 ethylene oxide units. In some cases, the polymer moiety is a discrete PEG comprising, e.g., about 28 ethylene oxide units. In some cases, the polymer moiety is a discrete PEG comprising, e.g., about 29 ethylene oxide units. In some cases, the polymer moiety is a discrete PEG comprising, e.g., about 30 ethylene oxide units. In some cases, the polymer moiety is a discrete PEG comprising, e.g., about 31 ethylene oxide units. In some cases, the polymer moiety is a discrete PEG comprising, e.g., about 32 ethylene oxide units. In some cases, the polymer moiety is a discrete PEG comprising, e.g., about 33 ethylene oxide units. In some cases, the polymer moiety is a discrete PEG comprising, e.g., about 34 ethylene oxide units. In some cases, the polymer moiety is a discrete PEG comprising, e.g., about 35 ethylene oxide units. In some cases, the polymer moiety is a discrete PEG comprising, e.g., about 36 ethylene oxide units. In some cases, the polymer moiety is a discrete PEG comprising, e.g., about 37 ethylene oxide units. In some cases, the polymer moiety is a discrete PEG comprising, e.g., about 38 ethylene oxide units. In some cases, the polymer moiety is a discrete PEG comprising, e.g., about 39 ethylene oxide units. In some cases, the polymer moiety is a discrete PEG comprising, e.g., about 40 ethylene oxide units. In some cases, the polymer moiety is a discrete PEG comprising, e.g., about 41 ethylene oxide units. In some cases, the polymer moiety is a discrete PEG comprising, e.g., about 42 ethylene oxide units. In some cases, the polymer moiety is a discrete PEG comprising, e.g., about 43 ethylene oxide units. In some cases, the polymer moiety is a discrete PEG comprising, e.g., about 44 ethylene oxide units. In some cases, the polymer moiety is a discrete PEG comprising, e.g., about 45 ethylene oxide units. In some cases, the polymer moiety is a discrete PEG comprising, e.g., about 46 ethylene oxide units. In some cases, the polymer moiety is a discrete PEG comprising, e.g., about 47 ethylene oxide units. In some cases, the polymer moiety is a discrete PEG comprising, e.g., about 48 ethylene oxide units.

In some cases, the polymer moiety is dPEG® (Quanta Biodesign Ltd).

In some aspects, the polymer moiety comprises a cationic mucic acid-based polymer (cMAP). In some instances, cMAP comprises one or more subunits of at least one repeating subunit, and the subunit structure is represented as Formula (V):

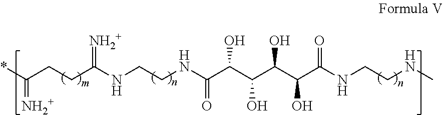

wherein m is independently at each occurrence 1, 2, 3, 4, 5, 6, 7, 8, 9, or 10, preferably 4-6 or 5; and n is independently at each occurrence 1, 2, 3, 4, or 5. In some aspects, m and n are, for example, about 10.

In some instances, cMAP is further conjugated to a PEG moiety, generating a cMAP-PEG copolymer, an mPEG-cMAP-PEGm triblock polymer, or a cMAP-PEG-cMAP triblock polymer. In some instances, the PEG moiety is in a range of from about 500 Da to about 50,000 Da. In some instances, the PEG moiety is in a range of from about 500 Da to about 1000 Da, greater than 1000 Da to about 5000 Da, greater than 5000 Da to about 10,000 Da, greater than 10,000 to about 25,000 Da, greater than 25,000 Da to about 50,000 Da, or any combination of two or more of these ranges.

In some instances, the polymer moiety is a cMAP-PEG copolymer, an mPEG-cMAP-PEGm triblock polymer, or a cMAP-PEG-cMAP triblock polymer. In some cases, the polymer moiety is cMAP-PEG copolymer. In other cases, the polymer moiety is an mPEG-cMAP-PEGm triblock polymer. In additional cases, the polymer moiety is a cMAP-PEG-cMAP triblock polymer.

In some aspects, the polymer moiety is conjugated to the polynucleic acid molecule, the binding moiety, and optionally to the endosomolytic moiety as illustrated supra.

### Endosomolytic or Cell Membrane Penetration Moiety

In some aspects, the conjugate described herein further comprises an additional conjugating moiety. In some instances, the additional conjugating moiety is an endosomolytic moiety and/or a cell membrane penetration moiety. In some cases, the endosomolytic moiety is a cellular compartmental release component, such as a compound capable of releasing from any of the cellular compartments known in the art, such as the endosome, lysosome, endoplasmic reticulum (ER), Golgi apparatus, microtubule, peroxisome, or other vesicular bodies with the cell. In some cases, the endosomolytic moiety comprises an endosomolytic polypeptide, an endosomolytic polymer, an endosomolytic lipid, or an endosomolytic small molecule. In some cases, the endosomolytic moiety comprises an endosomolytic polypeptide. In other cases, the endosomolytic moiety comprises an endosomolytic polymer. In some cases, the cell membrane penetration moiety comprises a cell penetrating peptide (CPP). In other cases, the cell membrane penetration moiety comprises a cell penetrating lipid. In other cases, the cell membrane penetration moiety comprises a cell penetrating small molecule.

### Endosomolytic and Cell Membrane Penetration Polypeptides

In some aspects, the conjugate described herein is further conjugated with an endosomolytic polypeptide. In some cases, the endosomolytic polypeptide is a pH-dependent membrane active peptide. In some cases, the endosomolytic polypeptide is an amphipathic polypeptide. In additional cases, the endosomolytic polypeptide is a peptidomimetic. In some instances, the endosomolytic polypeptide comprises INF, melittin, meucin, or their respective derivatives thereof. In some instances, the endosomolytic polypeptide comprises INF or its derivatives thereof. In other cases, the endosomolytic polypeptide comprises melittin or its derivatives thereof. In additional cases, the endosomolytic polypeptide comprises meucin or its derivatives thereof.

In some instances, INF7 is a polypeptide comprising CGIFGEIEELIEEGLENLIDWGNA (SEQ ID NO: 298), or GLFEAIEGFIENGWEGMIDGWYGC (SEQ ID NO: 299). In some instances, INF7 or its derivatives comprise a sequence of GLFEAIEGFIENGWEGMIWDYGSGSCG (SEQ ID NO: 300), GLFEAIEGFIENGWEGMIDG WYG-(PEG)6-NH2 (SEQ ID NO: 301), or GLFEAIEGFIENGWEGMIWDYG-SGSC-K(GalNAc)2 (SEQ ID NO: 302).

In some cases, melittin is a polypeptide comprising CLIGAILKVLATGLPTLISWIKNKRKQ (SEQ ID NO: 303), or GIGAVLKVLTTGLPALISWIKRKRQQ (SEQ ID NO: 304). In some instances, melittin comprises a polypeptide sequence as described in U.S. Pat. No. 8,501,930.

In some instances, meucin is an antimicrobial peptide (AMP) derived from the venom gland of the scorpion Mesobuthus eupeus. In some instances, meucin comprises of meucin-13 those sequence comprises IFGAIAGLLKNIF-NH₂ (SEQ ID NO: 305) and meucin-18 those sequence comprises FFGHLFKLATKIIPSLFQ (SEQ ID NO: 306).

In some instances, the endosomolytic polypeptide comprises a polypeptide in which its sequence is at least 50%, 60%, 70%, 80%, 90%, 95%, or 99% sequence identity to INF7 or its derivatives thereof, melittin or its derivatives thereof, or meucin or its derivatives thereof. In some instances, the endosomolytic moiety comprises INF7 or its derivatives thereof, melittin or its derivatives thereof, or meucin or its derivatives thereof.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 298-302. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 298. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 299-302. In some cases, the endosomolytic moiety comprises SEQ ID NO: 298. In some cases, the endosomolytic moiety comprises SEQ ID NO: 299-302. In some cases, the endosomolytic moiety consists of SEQ ID NO: 298. In some cases, the endosomolytic moiety consists of SEQ ID NO: 299-302.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 303 or 304. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 303. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 304. In some cases, the endosomolytic moiety comprises SEQ ID NO: 303. In some cases, the endosomolytic moiety comprises SEQ ID NO: 304. In some cases, the endosomolytic moiety consists of SEQ ID NO: 303. In some cases, the endosomolytic moiety consists of SEQ ID NO: 304.

In some instances, the endosomolytic moiety is meucin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 305 or 306. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 305. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 306. In some cases, the endosomolytic moiety comprises SEQ ID NO: 305. In some cases, the endosomolytic moiety comprises SEQ ID NO: 306. In some cases, the endosomolytic moiety consists of SEQ ID NO: 305. In some cases, the endosomolytic moiety consists of SEQ ID NO: 306.

In some instances, the endosomolytic moiety comprises a sequence as illustrated in Table 8.

TABLE 8

| NAME | ORIGIN | AMINO ACID SEQUENCE | SEQ ID NO: | TYPE |
|---|---|---|---|---|
| Pep-1 | NLS from | KETWWETWWTEWSQPKKKRKV | 307 | Primary |
| | Simian Virus 40 | | | amphipathic |
| | large antigen | | | |
| | and | | | |
| | Reverse | | | |
| | transcriptase of | | | |
| | HIV | | | |
| pVEC | VE-cadherin | LLIILRRRRIRKQAHAHSK | 308 | Primary |
| | | | | amphipathic |
| VT5 | Synthetic | DPKGDPKGVTVTVTVTVTGKGDP | 309 | ß-sheet |
| | peptide | KPD | | amphipathic |
| C105Y | 1-antitrypsin | CSIPPEVKFNKPFVYLI | 310 | — |
| Transportan | Galanin and | GWTLNSAGYLLGKINLKALAALA | 311 | Primary |
| | mastoparan | KKIL | | amphipathic |
| TP10 | Galanin and | AGYLLGKINLKALAALAKKIL | 312 | Primary |
| | mastoparan | | | amphipathic |
| MPG | A hydrophobic | GALFLGFLGAAGSTMGA | 313 | ß-sheet |
| | domain from the | | | amphipathic |
| | fusion sequence | | | |
| | of HIV gp41 | | | |
| | and NLS of | | | |
| | SV40 T antigen | | | |
| gH625 | Glycoprotein gH | HGLASTLTRWAHYNALIRAF | 314 | Secondary |
| | of HSV type I | | | amphipathic α- |
| | | | | helical |
| CADY | PPTG1 peptide | GLWRALWRLLRSLWRLLWRA | 315 | Secondary |
| | | | | amphipathic α- |
| | | | | helical |
| GALA | Synthetic | WEAALAEALAEALAEHLAEALAE | 316 | Secondary |
| | peptide | ALEALAA | | amphipathic α- |
| | | | | helical |
| INF | Influenza HA2 | GLFEAIEGFIENGWEGMIDGWYG | 317 | Secondary |
| | fusion peptide | C | | amphipathic α- |
| | | | | helical/pH- |
| | | | | dependent |
| | | | | membrane active |
| | | | | peptide |
| HA2E5-TAT | Influenza HA2 | GLFGAIAGFIENGWEGMIDGWYG | 318 | Secondary |
| | subunit of | | | amphipathic α- |
| | influenza virus | | | helical/pH- |
| | X31 strain | | | dependent |
| | fusion peptide | | | membrane active |
| | | | | peptide |
| HA2- | Influenza HA2 | GLFGAIAGFIENGWEGMIDGRQIKI | 319 | pH-dependent |
| penetratin | subunit of | WFQNRRMKW | | membrane active |
| | influenza virus | KK-amide | | peptide |
| | X31 strain | | | |
| | fusion peptide | | | |
| HA-K4 | Influenza HA2 | GLFGAIAGFIENGWEGMIDG- | 320 | pH-dependent |
| | subunit of | SSKKKK | | membrane active |
| | influenza virus | | | peptide |
| | X31 strain | | | |
| | fusion peptide | | | |
| HA2E4 | Influenza HA2 | GLFEAIAGFIENGWEGMIDGGGYC | 321 | pH-dependent |
| | subunit of | | | membrane active |
| | influenza virus | | | peptide |
| | X31 strain | | | |
| | fusion peptide | | | |
| H5WYG | HA2 analogue | GLFHAIAHFIHGGWH | 322 | pH-dependent |
| | | GLIHGWYG | | membrane active |
| | | | | peptide |
| GALA-INF3- | INF3 fusion | GLFEAIEGFIENGWEGLAEALAEA | 323 | pH-dependent |
| (PEG)6-NH | peptide | LEALAA- | | membrane active |
| | | (PEG)6-NH2 | | peptide |
| CM18-TAT11 | Cecropin-A- | KWKLFKKIGAVLKVLTTG- | 324 | pH-dependent |
| | Melittin2-12 | YGRKKRRQRRR | | membrane active |
| | (CM18) fusion | | | peptide |
| | peptide | | | |

In some cases, the endosomolytic moiety comprises a Bak BH3 polypeptide which induces apoptosis through antagonization of suppressor targets such as Bcl-2 and/or Bcl-xL. In some instances, the endosomolytic moiety comprises a Bak BH3 polypeptide described in Albarran, et al., “Efficient intracellular delivery of a pro-apoptotic peptide with a pH-responsive carrier,” Reactive & Functional Polymers 71: 261-265 (2011).

In some instances, the endosomolytic moiety comprises a polypeptide (e.g., a cell-penetrating polypeptide) as described in PCT Publication Nos. WO2013/166155 or WO2015/069587.

### Endosomolytic Lipids

In some aspects, the endosomolytic moiety is a lipid (e.g., a fusogenic lipid). In some aspects, the conjugate described herein is further conjugated with an endosomolytic lipid (e.g., fusogenic lipid). Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-o1 (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC).

In some instances, an endosomolytic moiety is a lipid (e.g., a fusogenic lipid) described in PCT Publication No. WO09/126,933.

### Endosomolytic Small Molecules

In some aspects, the endosomolytic moiety is a small molecule. In some aspects, the conjugate described herein is further conjugated with an endosomolytic small molecule. Exemplary small molecules suitable as endosomolytic moieties include, but are not limited to, quinine, chloroquine, hydroxychloroquines, amodiaquins (camoquines), amopyroquines, primaquines, mefloquines, nivaquines, halofantrines, quinone imines, or a combination thereof. In some instances, quinoline endosomolytic moieties include, but are not limited to, 7-chloro-4-(4-diethylamino-1-methylbutyl-amino)quinoline (chloroquine); 7-chloro-4-(4-ethyl-(2-hydroxyethyl)-amino-1-methylbutyl-amino)quinoline (hydroxychloroquine); 7-fluoro-4-(4-diethylamino-1-methylbutyl-amino)quinoline; 4-(4-diethylamino-1-methylbutylamino) quinoline; 7-hydroxy-4-(4-diethyl-amino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethyl-amino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethyl-amino-1-butylamino)quinoline; 4-(1-carboxy-4-diethylamino-1-butylamino) quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethyl-amino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxy-ethyl)-amino-1-methylbutylamino-)quinoline; 7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl-1)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino) quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha, alpha′-bis(2-methyl-1-pyrrolidinyl)-2,5-xylidinoquinoline, 4-[(4-diethyl-amino)-1-methylbutyl-amino]-6-methoxyquinoline; 3-fluoro-4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha, alpha′-bis(2-methyl-1-pyrrolidinyl)-2,5-xylidinoquinoline; 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof. In some instances, an endosomolytic moiety is a small molecule described in Naisbitt et al (1997, J Pharmacol Exp Therapy 280:884-893) and in U.S. Pat. No. 5,736,557.

### Cell Penetrating Polypeptide (CPP)

In some aspects, cell penetrating polypeptide comprises positively charged short peptides with 5-30 amino acids. In some aspects, cell penetrating polypeptide comprises arginine or lysine rich amino acid sequences. In some aspects, cell penetrating polypeptide includes any polypeptide or combination thereof listed in Table 9.

TABLE 9

| Peptide | Sequence | SEQ ID NO: |
|---|---|---|
| Antennapedia Penetratin (43-58) | RQIKIWFQNRRMKWKK | 325 |
| HIV-1 TAT protein (48-60) | GRKKRRQRRRPPQ | 326 |
| pVEC Cadherin (615-632) | LLIILRRRIRKQAHAHSK | 327 |
| Transportan Galanine/Mastoparan | GWTLNSAGYLLGKINLKALAALAKKIL | 328 |
| MPG HIV-gp41/SV40 T-antigen | GALFLGFLGAAGSTMGAWSQPKKKRKV | 329 |
| Pep-1 HIV-reverse | KETWWETWWTEWSQPKKKRKV | 330 |
| transcriptase/SV40 T-antigen | | |
| Polyarginines | R(n); 6 < n < 12 | 331 |
| MAP | KLALKLALKALKAALKLA | 332 |
| R6W3 | RRWWRRWRR | 333 |
| NLS | CGYGPKKKRKVGG | 334 |
| 8-lysines | KKKKKKKK | 335 |
| ARF (1-22) | MVRRFLVTLRIRRACGPPRVRV | 336 |
| Azurin-p28 | LSTAADMQGVVTDGMASGLDKDYLKPDD | 337 |

### Linkers

In some aspects, a linker described herein is a cleavable linker or a non-cleavable linker. In some instances, the linker is a cleavable linker. In other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some instances, the non-polymeric linker does not encompass a polymer that is described above. In some instances, the non-polymeric linker does not encompass a polymer encompassed by the polymer moiety. In some cases, the non-polymeric linker does not encompass a polyalkylene oxide (e.g., PEG). In some cases, the non-polymeric linker does not encompass a PEG.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[B-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some instances, the linker comprises a reactive functional group. In some cases, the reactive functional group comprises a nucleophilic group that is reactive to an electrophilic group present on a binding moiety. Exemplary electrophilic groups include carbonyl groups-such as aldehyde, ketone, carboxylic acid, ester, amide, enone, acyl halide or acid anhydride. In some aspects, the reactive functional group is aldehyde. Exemplary nucleophilic groups include hydrazide, oxime, amino, hydrazine, thiosemicarbazone, hydrazine carboxylate, and arylhydrazide.

In some aspects, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some aspects, the linker comprises a peptide moiety. In some instances, the peptide moiety comprises at least 2, 3, 4, 5, or 6 more amino acid residues. In some instances, the peptide moiety comprises at most 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 338), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 339), or Gly-Phe-Leu-Gly (SEQ ID NO: 340). In some instances, the linker comprises a peptide moiety such as: Val-Cit (valine-citrulline), Gly-Gly-Phe-Gly (SEQ ID NO: 338), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 339), or Gly-Phe-Leu-Gly (SEQ ID NO: 340). In some cases, the linker comprises Val-Cit. In some cases, the linker is Val-Cit.

In some aspects, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some aspects, the linker comprises one or more of a maleimide group, a peptide moiety, and/or a benzoic acid group, in any combination. In some aspects, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some aspects, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication NO. WO2015038426.

In some aspects, the linker is a dendritic type linker. In some instances, the dendritic type linker comprises a branching, multifunctional linker moiety. In some instances, the dendritic type linker is used to increase the molar ratio of polynucleotide to the binding moiety. In some instances, the dendritic type linker comprises PAMAM dendrimers.

In some aspects, the linker is a traceless linker or a linker in which after cleavage does not leave behind a linker moiety (e.g., an atom or a linker group) to a binding moiety, a polynucleotide, a polymer, or an endosomolytic moiety. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker is a linker described in U.S. Pat. Nos. 6,884,869; 7,498,298; 8,288,352; 8,609,105; or 8,697,688; U.S. Patent Publication NOs. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

In some instances, a binding moiety is conjugated to a polynucleotide via a first linker. In some instances, a polymer is conjugated to a polynucleotide via a second linker. In some aspects, the first linker and the second linker are each independently a bond or a non-polymeric linker. In some instances, the first linker and the second linker are each independently a bond. In some cases, the first linker and the second linker are each independently a non-polymeric linker.

In some instances, the first linker is a bond or a non-polymeric linker. In some instances, the first linker is a bond. In some instances, the first linker is a non-polymeric linker. In some instances, the first linker is a C₁-C₆ alkyl group. In some cases, the first linker is a C₁-C₆ alkyl group, such as for example, a C₅, C₄, C₃, C₂, or C₁ alkyl group. In some cases, the C₁-C₆ alkyl group is an unsubstituted C₁-C₆ alkyl group. As used in the context of a linker, and in particular in the context of the first linker, alkyl means a saturated straight or branched hydrocarbon radical containing up to six carbon atoms. In some instances, the first linker includes a homobifunctional linker or a heterobifunctional linker described supra. In some cases, the first linker includes a heterobifunctional linker. In some cases, the first linker includes sMCC. In other instances, the first linker includes a heterobifunctional linker optionally conjugated to a C₁-C₆ alkyl group. In other instances, the first linker includes sMCC optionally conjugated to a C₁-C₆ alkyl group. In additional instances, the first linker does not include a homobifunctional linker or a heterobifunctional linker described supra.

In some instances, the second linker is a bond or a linker. In some instances, the second linker is a bond. In other cases, the second linker is a polymeric linker. In additional cases, the second linker is a non-polymeric linker. In some aspects, the second linker 2 is a C₁-C₆ alkyl group. In some instances, the second linker is a homobifunctional linker or a heterobifunctional linker described supra. In some instances, the second linker is a homobifunctional linker described supra. In some instances, the second linker is a heterobifunctional linker described supra. In some instances, the second linker comprises a maleimide group, such as maleimidocaproyl (mc) or a self-stabilizing maleimide group described above. In some instances, the second linker comprises a peptide moiety, such as Val-Cit. In some instances, the second linker comprises a benzoic acid group, such as PABA. In additional instances, the second linker comprises a combination of a maleimide group, a peptide moiety, and/or a benzoic acid group. In additional instances, the second linker comprises a me group. In additional instances, the second linker comprises a mc-val-cit group. In additional instances, the second linker comprises a val-cit-PABA group. In additional instances, the second linker comprises a mc-val-cit-PABA group.

### Methods of Use

Muscle atrophy refers to a loss of muscle mass and/or to a progressive weakening and degeneration of muscles. In some cases, the loss of muscle mass and/or the progressive weakening and degeneration of muscles occurs due to a high rate of protein degradation, a low rate of protein synthesis, or a combination of both. In some cases, a high rate of muscle protein degradation is due to muscle protein catabolism (i.e., the breakdown of muscle protein in order to use amino acids as substrates for gluconeogenesis).

In one embodiment, muscle atrophy refers to a significant loss in muscle strength. By significant loss in muscle strength is meant a reduction of strength in diseased, injured, or unused muscle tissue in a subject relative to the same muscle tissue in a control subject. In an embodiment, a significant loss in muscle strength is a reduction in strength of at least 10%, at least 15%, at least 20%, at least 25%, at least 30%, at least 35%, at least 40%, at least 45%, at least 50%, or more relative to the same muscle tissue in a control subject. In another embodiment, by significant loss in muscle strength is meant a reduction of strength in unused muscle tissue relative to the muscle strength of the same muscle tissue in the same subject prior to a period of nonuse. In an embodiment, a significant loss in muscle strength is a reduction of at least 10%, at least 15%, at least 20%, at least 25%, at least 30%, at least 35%, at least 40%, at least 45%, at least 50%, or more relative to the muscle strength of the same muscle tissue in the same subject prior to a period of nonuse.

In some aspects, described herein is a method of treating cardiomyopathy in a subject, which comprises providing polynucleic acid molecule or polynucleic acid molecule conjugate described herein and administering to the subject a therapeutically effective amount of a polynucleic acid molecule described herein or a polynucleic acid molecule conjugate described herein. In some instances, the polynucleic acid molecule or polynucleic acid molecule conjugate is effective to reduce a quantity of the mRNA transcript of human PRKAG2. In some instances, the subject is diagnosed or suspected to have cardiomyopathy. In some instances, the subject has suffered, or is suffering from one or more symptoms of cardiomyopathy. In some instances, the polynucleic acid molecule or polynucleic acid molecule conjugate mediates RNA interference against the human PRKAG2 mRNA as to modulate or treat cardiomyopathy (and/or its symptoms thereof) caused by or associated with a glycogen storage disease in a subject. In some aspects, the polynucleic acid molecule or polynucleic acid molecule conjugate mediates RNA interference against a human PRKAG2 mRNA comprising a mutation. In some instances, the mutation is a point mutation of the human PRKAG2 gene as to modulate cardiomyopathy caused by glycogen storage disease in a subject. In some instances, the subject is a human.

In some aspects, described herein is a method of treating PRKAG2 cardiac syndrome in a subject, which comprises providing polynucleic acid molecule or polynucleic acid molecule conjugate described herein and administering to the subject a therapeutically effective amount of a polynucleic acid molecule or a polynucleic acid molecule conjugate described herein. In some instances, the polynucleic acid molecule or polynucleic acid molecule conjugate is effective to reduce a quantity of the mRNA transcript of human PRKAG2. In some instances, the subject is diagnosed or suspected to have PRKAG2 cardiac syndrome. In some instances, the subject has suffered, or is suffering from one or more symptoms of PRKAG2 cardiac syndrome. In some instances, the polynucleic acid molecule or polynucleic acid molecule conjugate mediates RNA interference against the human PRKAG2 mRNA as to modulate or treat PRKAG2 cardiac syndrome in a subject. In some aspects, the polynucleic acid molecule or polynucleic acid molecule conjugate mediates RNA interference against a human PRKAG2 mRNA comprising a mutation. In some instances, the mutation is a point mutation of the human PRKAG2 gene as to modulate PRKAG2 cardiac syndrome in a subject. In some instances, the subject is a human.

In some aspects, described herein is a method of treating cardiomyopathy in a subject, which comprises providing an siRNA-antibody conjugate (siRNA conjugate) described herein and administering to the subject a therapeutically effective amount of the siRNA-antibody conjugate described herein and reducing the levels of PRKAG2 mRNA transcript of human PRKAG2 in said subject. In some instances, cardiomyopathy is cardiomyopathy caused by a glycogen storage disease. In some instances, cardiomyopathy is cardiomyopathy caused by PRKAG2 syndrome or PRKAG2 cardiac syndrome. In some instances, the siRNA-conjugate mediates RNA interference against the human PRKAG2 mRNA to reduce the levels of mRNA transcript of human PRKAG2 mRNA in the subject, thereby treats cardiomyopathy caused PRKAG2 syndrome or cardiac syndrome, or its symptoms thereof, in the subject. In some instances, the siRNA-conjugate mediates RNA interference against a mutated PRKAG2 variant comprising a point mutation within the PRKAG2 gene to reduce the levels of mRNA transcript of human PRKAG2 in said subject, thereby treating cardiomyopathy caused by PRKAG2 syndrome in the subject.

In some aspects, described herein is a method of treating PRKGA2 cardiac syndrome in a subject, which comprises providing an siRNA-antibody conjugate (siRNA conjugate) described herein and administering to the subject a therapeutically effective amount of the siRNA-antibody conjugate described herein and reducing the levels of PRKAG2 mRNA transcript of human PRKAG2 in said subject. In some instances, the siRNA-conjugate mediates RNA interference to reduce the levels of mRNA transcript of human PRKAG2 mRNA in the subject thereby treats PRKGA2 cardiac syndrome in the subject. In some instances, the siRNA-conjugate mediates RNA interference against a mutated PRKAG2 variant comprising a point mutation within the PRKAG2 gene to reduce the levels of mRNA transcript of human PRKAG2 mRNA comprising the mutation, thereby treats PRKGA2 cardiac syndrome in the subject.

In some aspects, described herein is a method of alleviating symptoms in a subject with cardiomyopathy, which comprises providing a PRKAG2 siRNA-antibody conjugate (PRKAG2-siRNA conjugate or PRKAG2-AOC) described herein and administering to the subject a therapeutically effective amount of the siRNA-antibody conjugate described herein by reducing the levels of mRNA transcript of human PRKAG2. In some instances, the cardiomyopathy is caused by a glycogen storage disease. In another embodiments, described herein is a method of alleviating symptoms in a subject with cardiomyopathy caused PRKAG2 syndrome or PRKAG2 cardiac syndrome by providing an siRNA-antibody conjugate described herein and administering to the patient with cardiomyopathy caused by PRKAG2 syndrome a therapeutically effective amount of the siRNA-antibody conjugate describes herein by targeting a PRKAG2 mutant comprising a point mutation within PRKAG2 gene and reducing the levels of mRNA transcript of human PRKAG2 comprising the mutation or reducing the levels of PRKAG2 protein comprising a mutation.

The symptoms of cardiomyopathy are related to the thickening of the heart muscle or cardiac muscles (e.g., hypertrophied cardiac muscles). In some instances, the symptoms of cardiomyopathy include arrhythmia (irregular heart rate or rhythm). In some instances, the symptoms of cardiomyopathy include chest pain, especially during activity. In some instances, the symptoms of cardiac hypertrophy include fatigue. In some instances, the symptoms of cardiomyopathy include fluttering or pounding feeling in the chest. In some instances, the symptoms of cardiomyopathy include heart murmur. In some instances, the symptoms of cardiomyopathy include lightheadedness or dizziness. In some instances, the symptoms of cardiomyopathy include fainting. In some instances, the symptoms of cardiomyopathy include shortness of breath, especially during activity.

In some aspects, described herein is a method of improving cardiac muscle functions in a patient by administering to the cardiomyopathy patient a therapeutically effective amount of the siRNA conjugate described herein thereby reducing the levels of mRNA transcript of human PRKAG2 or reducing the levels of PRKAG2 protein. In some instances, cardiomyopathy is caused by a glycogen storage disease. In some instances, cardiomyopathy is caused by PRKAG2 syndrome or PRKAG2 cardiac syndrome. In some aspects, described herein is a method of improving cardiac muscle functions or alleviating cardiomyopathy symptoms as described above a patient suffering from cardiomyopathy by administering to the cardiomyopathy caused by PRKAG2 syndrome patient a therapeutically effective amount of the siRNA conjugate described herein thereby reducing the levels of mRNA transcript of human PRKAG2 or reducing the levels of PRKAG2 protein. In some instances, the siRNA molecule reduces the mRNA expression level of the PRKAG2 mRNA in the muscle cell at least 40%, 50%, 60% compared to PRKAG2 mRNA level before administration.

In some aspects, described herein is a method of treating cardiomyopathy in a subject, which comprises providing an antisense oligonucleotide (ASO) antibody conjugate (ASO-conjugate) described herein and administering to the subject a therapeutically effective amount of the ASO-conjugate described herein and reducing the levels of PRKAG2 mRNA transcript of human PRKAG2 in said subject. In some instances, cardiomyopathy is caused by or associated with a mutated PRKAG2. In some instances, the mutated PRKAG2 comprises R531G mutation. In some instances, the ASO-conjugate mediates RNA interference against the human PRKAG2 mRNA to reduce the levels of mRNA transcript of human PRKAG2 in said subject, thereby treats cardiomyopathy caused by or associated with a mutated PRKAG2 in the subject. In some instances, the ASO-conjugate mediates RNA interference against a PRKAG2 mutant comprising a point mutation within the PRKAG2 gene to reduce the levels of mRNA transcript of human PRKAG2 with the mutation in said subject, thereby treats cardiomyopathy in the subject. In some instances, the ASO molecule reduces the mRNA expression level of the PRKAG2 mRNA in the muscle cell at least 40%, 50%, 60% compared to PRKAG2 mRNA level before administration.

In some aspects, described herein is a method of treating cardiomyopathy in a subject. In some instances, the subject with cardiomyopathy suffers from cardiomyopathy caused by a glycogen storage disease. In some instances, the subject with cardiomyopathy suffers from cardiomyopathy caused by PRKAG2 syndrome. In some instances, the subject suffers from PRKAG2 cardiac syndrome. In some instances, the subject with cardiomyopathy suffers from cardiomyopathy caused by a mutation of the PRKAG2 gene. In some instances, the subject with cardiomyopathy subject has muscle cells expressing a mutant PRKAG2 selected from a mutated PRKAG2 which comprises a single point mutation the PRKAG2 gene. In some aspects, the muscle cells are cardiac muscle cells.

In some aspects, described herein is a method of modulating PRKAG2 expression or activity in a muscle cell by contacting the muscle cell with a polynucleotide conjugate (siRNA-antibody conjugate or ASO-antibody conjugate) or a polynucleotide molecule (siRNA or ASO), thereby modulating PRKAG2 expression or activity in the muscle cell. In some instances, the polynucleotide conjugate or the polynucleotide molecule reduces the PRKAG2 expression or activity at least 20%, 30%, 40%, 50%, 60%, 70%, or 80% compared to untreated muscle cells, or before the treatment. In some instances, the effect of reduced PRKAG2 expression or activity is maintained for at least 3 days, 7 days, 14 days, 21 days, 28 days, 60 days, 90 days, 120 days, 5 months, 6 months by maintaining the reduced expression or activity of at least 20%, 30%, 40%, or 50%.

In some aspects, described herein is a method of inhibiting or reducing glycogen accumulation in a tissue or an organ of a subject by contacting the tissue or organ with or administering to the subject a polynucleotide conjugate (siRNA-antibody conjugate or ASO-antibody conjugate) or a polynucleotide molecule (siRNA or ASO) described herein. In some aspects, described herein is a method of inhibiting or reducing glycogen accumulation in a skeletal muscle tissue or a cardiac tissue (e.g., cardiac muscle) of a subject by contacting the skeletal muscle tissue or a cardiac tissue with or administering the subject a polynucleotide conjugate (siRNA-antibody conjugate or ASO-antibody conjugate) or a polynucleotide molecule (siRNA or ASO) described herein. The polynucleotide conjugate comprises an anti-transferrin receptor antibody or antigen-binding fragment thereof conjugated to a polynucleotide molecule, which hybridizes to a target sequence of PRKAG2 mRNA and mediates RNA interference against PRKAG2 mRNA. In some instances, the polynucleotide molecule reduces the mRNA expression level of the PRKAG2 mRNA in the muscle cell at least 40%, 50%, 60% compared to PRKAG2 mRNA level before administration or contacting.

In some aspects, described herein is a method of modulating PRKAG2 expression or activity in a subject (e.g., a patient) by administering the subject with a polynucleotide conjugate (siRNA-antibody conjugate or ASO-antibody conjugate) or a polynucleotide molecule (siRNA or ASO), thereby modulating PRKAG2 expression or activity in the subject. In some instances, the polynucleotide conjugate or the polynucleotide molecule reduces the PRKAG2 expression or activity at least 20%, 30%, 40%, 50%, 60%, 70%, or 80% in the muscle cells (e.g., heart muscle cells, skeletal muscle cells, etc.) in the subject compared to untreated subject, or before the treatment. In some instances, the effect of reduced PRKAG2 expression or activity is maintained for at least 3 days, 7 days, 14 days, 21 days, 28 days, 60 days, 90 days, 120 days, 5 months, 6 months by maintaining the reduced expression or activity of at least 20%, 30%, 40%, or 50%.

In some aspects, described herein is a use of the polynucleotide conjugate (e.g., siRNA-antibody conjugate or ASO-antibody conjugate) or polynucleotide molecule (e.g., siRNA or ASO) in the manufacture of a medicament for treating cardiomyopathy in a subject in need thereof. In some instances, the polynucleotide conjugate comprises an anti-transferrin receptor antibody or antigen-binding fragment thereof conjugated to a polynucleotide molecule. In some instances, the polynucleotide molecule hybridizes to a target sequence of PRKAG2 mRNA and mediates RNA interference against the PRKAG2 mRNA in a muscle cell. In some instances, the cardiomyopathy is caused by a glycogen storage disease. In some instances, the cardiomyopathy is caused by PRKAG2 syndrome or PRKAG2 cardiac syndrome. In some instances, the PRKAG2 syndrome or PRKAG2 cardiac syndrome is caused by a mutated PRKAG2 that has a gain of function. In some instances, the mutated PRKAG2 comprises a R531G mutation. In some instances, the polynucleotide conjugate is formulated for administration parentally, orally, intranasally, buccally, rectally, transdermally, intravenously, subcutaneously, or intrathecally. In some instances, the polynucleotide conjugate reduces the mRNA expression level of the PRKAG2 mRNA in the muscle cell at least 40%, 50%, or 60% compared to PRKAG2 mRNA level before administration. In some instances, the polynucleotide conjugate is administered in an effective amount, optionally the effective amount of the polynucleotide conjugate is effective to reduce the glycogen accumulation in a skeletal muscle tissue or a cardiac tissue of the subject. In some instances, the subject is a human.

In some aspects, described herein is a use of a polynucleotide conjugate (e.g., siRNA-antibody conjugate or ASO-antibody conjugate) or polynucleotide molecule (e.g., siRNA and ASO) in the manufacture of a medicament for inhibiting or reducing glycogen accumulation in a skeletal muscle tissue or a cardiac tissue of a subject in need thereof. In some instances, the polynucleotide conjugate comprises an anti-transferrin receptor antibody or antigen-binding fragment thereof conjugated to a polynucleotide molecule. In some instances, the polynucleotide molecule hybridizes to a target sequence of PRKAG2 mRNA and mediates RNA interference against the PRKAG2 mRNA. In some instances, the subject is affected by PRKAG2 syndrome or PRKAG2 cardiac syndrome. In some instances, the PRKAG2 syndrome or PRKAG2 cardiac syndrome is caused by a mutated PRKAG2 that has a gain of function. In some instances, the mutated PRKAG2 comprises R531G mutation. In some instances, the polynucleotide conjugate reduces the mRNA expression level of the PRKAG2 mRNA in the muscle cell at least 40%, 50%, or 60% compared to PRKAG2 mRNA level before administration.

In the methods and uses described herein, in some instances, the polynucleic acid molecules or polynucleic acid molecule conjugates (e.g., antibody-oligonucleotide conjugate (AOC)) are administered to the subject intravenously or subcutaneously at an effective dose to treat the cardiomyopathy or modulate PRKAG2 expression or activity in a subject. In some instances, the effective dose is administered to the subject by a single intravenous or subcutaneous administration. In some instances, the effective dose is administered to the subject by multiple intravenous or subcutaneous administration, which sum amount of the multiple injections amounts to the effective dose to treat the cardiomyopathy or modulate PRKAG2 expression or activity in a subject.

### Pharmaceutical Formulation

In some aspects, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition described herein is formulated for oral administration. In still other instances, the pharmaceutical composition described herein is formulated for intranasal administration.

In some aspects, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, u-chymotrypsin, polylysine, polyarginine, histone, protamine, ovalbumin or dextrin or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100 or more polynucleic acid molecules or binding moieties described herein are conjugated either directly or indirectly to a nanoparticle.

In some aspects, the pharmaceutical formulation comprises a delivery vector, e.g., a recombinant vector, the delivery of the polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide transient expression of polynucleic acid molecules.

In some aspects, the pharmaceutical formulation includes a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulation further includes pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulation further includes diluent which are used to stabilize compounds because they provide a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulation includes disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia*, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulation includes filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil*, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol has a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants are included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium docusate, triacetin, Tween 80, vitamin E TPGS, ammonium salts and the like.

### Therapeutic Regimens

In some aspects, the pharmaceutical compositions described herein are administered for therapeutic applications. In some aspects, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, once in two months, once in three months, once in four months, once in five months, once in six months or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some aspects, one or more pharmaceutical compositions are administered simultaneously, sequentially, or at an interval period of time. In some aspects, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some aspects, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5-hour, 1 hour, 2 hours, 3 hours, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some aspects, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some aspects, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

In some aspects, the polynucleotide conjugate described herein is administered parenterally, orally, intranasally, buccally, rectally, transdermally, intravenously, subcutaneously, or intrathecally. In some aspects, the polynucleotide conjugate described herein is administered intravenously or subcutaneously. In some aspects, the polynucleotide conjugate described herein is administered intravenously. In some aspects, the polynucleotide conjugate described herein is administered subcutaneously.

In some aspects, the polynucleotide conjugate described herein is administered with a dose of at least 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is administered with a dose of at most 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is administered with a dose of about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg. In some aspects, the polynucleotide conjugate described herein is administered with a dose of 1-10 mg/kg. In some aspects, the polynucleotide conjugate described herein is administered with a dose of 1-4 mg/kg. In some aspects, the polynucleotide conjugate described herein is administered with a dose of 1 mg/kg. In some aspects, the polynucleotide conjugate described herein is administered with a dose of 2 mg/kg.

In some aspects, the polynucleotide conjugate described herein is intravenously administered with a dose of at least 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is intravenously administered with a dose of at most 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is intravenously administered with a dose of about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg. In some aspects, the polynucleotide conjugate described herein is intravenously administered with a dose of 1-10 mg/kg. In some aspects, the polynucleotide conjugate described herein is administered with a dose of 1-4 mg/kg. In some aspects, the polynucleotide conjugate described herein is administered with a dose of 1 mg/kg. In some aspects, the polynucleotide conjugate described herein is administered with a dose of 2 mg/kg.

In some aspects, the polynucleotide conjugate described herein is subcutaneously administered with a dose of at least 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is subcutaneously administered with a dose of at most 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is subcutaneously administered with a dose of about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg. In some aspects, the polynucleotide conjugate described herein is subcutaneously administered with a dose of 1-10 mg/kg. In some aspects, the polynucleotide conjugate described herein is administered with a dose of 1-4 mg/kg. In some aspects, the polynucleotide conjugate described herein is administered with a dose of 1 mg/kg. In some aspects, the polynucleotide conjugate described herein is administered with a dose of 2 mg/kg.

In some aspects, the polynucleotide conjugate described herein is administered (e.g., intravenously or subcutaneously) with a dose of about 1, 2, 3, or 4 mg/kg. In some aspects, the polynucleotide conjugate described herein is intravenously administered with a dose of about 1 mg/kg. In some aspects, the polynucleotide conjugate described herein is intravenously administered with a dose of about 2 mg/kg. In some aspects, the polynucleotide conjugate described herein is intravenously administered with a dose of about 3 mg/kg. In some aspects, the polynucleotide conjugate described herein is intravenously administered with a dose of about 4 mg/kg. In some aspects, the polynucleotide conjugate described herein is subcutaneously administered with a dose of about 1 mg/kg. In some aspects, the polynucleotide conjugate described herein is subcutaneously administered with a dose of about 2 mg/kg. In some aspects, the polynucleotide conjugate described herein is subcutaneously administered with a dose of about 3 mg/kg. In some aspects, the polynucleotide conjugate described herein is subcutaneously administered with a dose of about 4 mg/kg.

In some aspects, the polynucleotide conjugate described herein is administered with a dose of at least 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is administered with a dose of at most 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is administered with a dose of about 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg. In some aspects, the polynucleotide conjugate described herein is intravenously administered with a dose of 1-10 mg siRNA/kg. In some aspects, the polynucleotide conjugate described herein is administered with a dose of 1-4 mg siRNA/kg. In some aspects, the polynucleotide conjugate described herein is administered with a dose of 1 mg siRNA/kg. In some aspects, the polynucleotide conjugate described herein is administered with a dose of 2 mg siRNA/kg.

In some aspects, the polynucleotide conjugate described herein is intravenously administered with a dose of at least 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is intravenously administered with a dose of at most 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is intravenously administered with a dose of about 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg. In some aspects, the polynucleotide conjugate described herein is intravenously administered with a dose of 1-10 mg siRNA/kg. In some aspects, the polynucleotide conjugate described herein is intravenously administered with a dose of 1-4 mg siRNA/kg. In some aspects, the polynucleotide conjugate described herein is intravenously administered with a dose of 1 mg siRNA/kg. In some aspects, the polynucleotide conjugate described herein is intravenously administered with a dose of 2 mg siRNA/kg.

In some aspects, the polynucleotide conjugate described herein is subcutaneously administered with a dose of at least 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is subcutaneously administered with a dose of at most 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg.

In some aspects, the polynucleotide conjugate described herein is subcutaneously administered with a dose of about 0.01 mg/kg (siRNA dose), 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6.0 mg/kg. In some aspects, the polynucleotide conjugate described herein is subcutaneously administered with a dose of 1-10 mg siRNA/kg. In some aspects, the polynucleotide conjugate described herein is subcutaneously administered with a dose of 1-4 mg siRNA/kg. In some aspects, the polynucleotide conjugate described herein is subcutaneously administered with a dose of 1 mg siRNA/kg. In some aspects, the polynucleotide conjugate described herein is administered with a dose of 2 mg siRNA/kg.

In some aspects, the polynucleotide conjugate described herein is administered (e.g., intravenously or subcutaneously) with a dose of about 1, 2, 3, or 4 mg/kg (siRNA dose). As used herein, siRNA dose refers the dose measured by the amount of siRNA in the conjugates. For example 1 mg/kg siRNA dose means 1 mg siRNA per kg of the subject body weight. Also, as used herein, “mg siRNA/kg” is interchangeably used with “mg/kg siRNA dose”. For example both “1 mg siRNA/kg” and “1 mg/kg siRNA dose” mean 1 mg siRNA per kg of the subject body weight. In some aspects, the polynucleotide conjugate described herein is intravenously administered with a dose of about 1 mg/kg (siRNA dose). In some aspects, the polynucleotide conjugate described herein is intravenously administered with a dose of about 2 mg/kg (siRNA dose). In some aspects, the polynucleotide conjugate described herein is intravenously administered with a dose of about 3 mg/kg (siRNA dose). In some aspects, the polynucleotide conjugate described herein is intravenously administered with a dose of about 4 mg/kg (siRNA dose). In some aspects, the polynucleotide conjugate described herein is subcutaneously administered with a dose of about 1 mg/kg (siRNA dose). In some aspects, the polynucleotide conjugate described herein is subcutaneously administered with a dose of about 2 mg/kg (siRNA dose). In some aspects, the polynucleotide conjugate described herein is subcutaneously administered with a dose of about 3 mg/kg (siRNA dose). In some aspects, the polynucleotide conjugate described herein is subcutaneously administered with a dose of about 4 mg/kg (siRNA dose).

In some aspects, the polynucleotide conjugate described herein is administered (e.g., intravenously or subcutaneously) with a single dose or multiple doses. In some aspects, the polynucleotide conjugate described herein is administered with a single dose. In some aspects, the polynucleotide conjugate described herein is administered with multiple doses.

In some aspects, the polynucleotide conjugate described herein is administered every 1, 2, 3, 4, 5, 6, 7, 8, 9, 10, 11, 12 weeks. In some aspects, the polynucleotide conjugate described herein is administered every 1, 2, 3, 4, 5, 6, 7, 8, 9, 10, 11, 12 weeks. In some aspects, the polynucleotide conjugate described herein is administered every 6 weeks. In some aspects, the polynucleotide conjugate described herein is administered every 9 weeks. In some aspects, the polynucleotide conjugate described herein is administered every 12 weeks.

In some aspects, the polynucleotide conjugate described herein is administered for at least 1, 2, 3, 4, 5, 6, 7, 8, 9, 10, 11, 12, 13, 14, 15, 16, 17, 18, 19, or 20 months. In some aspects, the polynucleotide conjugate described herein is administered for at most 1, 2, 3, 4, 5, 6, 7, 8, 9, 10, 11, 12, 13, 14, 15, 16, 17, 18, 19, or 20 months. In some aspects, the polynucleotide conjugate described herein is administered for about 1, 2, 3, 4, 5, 6, 7, 8, 9, 10, 11, 12, 13, 14, 15, 16, 17, 18, 19, or 20 months. In some aspects, the polynucleotide conjugate described herein is administered for 1, 2, 3, 4, 5, 6, 7, 8, 9, 10, 11, 12, 13, 14, 15, 16, 17, 18, 19, or 20 months.

In some aspects, the polynucleotide conjugate described herein is administered for about 1-18 months. In some aspects, the polynucleotide conjugate described herein is administered for about 1-12 months. In some aspects, the polynucleotide conjugate described herein is administered for about 1-6 months. In some aspects, the polynucleotide conjugate described herein is administered for about 6-12 months. In some aspects, the polynucleotide conjugate described herein is administered for about 12-18 months.

In some embodiments, dosing amount and/or dosing schedule may be adjusted during treatment.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include target nucleic acid molecule described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

The section headings used herein are for organizational purposes only and are not to be construed as limiting the subject matter described.

The abbreviations used herein have their conventional meaning within the chemical and biological arts. The chemical structures and formulae set forth herein are constructed according to the standard rules of chemical valency known in the chemical arts.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

As used herein, the term “polynucleic acid” is interchangeably used with the term “oligonucleotide” or “polynucleotide_”

As used herein, the terms “individual(s)”, “subject(s)” and “patient(s)” mean any mammal. In some aspects, the mammal is a human. In some aspects, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The term “therapeutically effective amount” relates to an amount of a polynucleic acid molecule conjugate that is sufficient to provide a desired therapeutic effect in a mammalian subject. In some cases, the amount is single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. Naturally, dosage levels of the particular polynucleic acid molecule conjugate employed to provide a therapeutically effective amount vary in dependence of the type of injury, the age, the weight, the gender, the medical condition of the subject, the severity of the condition, the route of administration, and the particular inhibitor employed. In some instances, therapeutically effective amounts of polynucleic acid molecule conjugate, as described herein, is estimated initially from cell culture and animal models. For example, IC₅₀ values determined in cell culture methods optionally serve as a starting point in animal models, while IC₅₀ values determined in animal models are optionally used to find a therapeutically effective dose in humans.

Skeletal muscle, or voluntary muscle, is generally anchored by tendons to bone and is generally used to effect skeletal movement such as locomotion or in maintaining posture. Although some control of skeletal muscle is generally maintained as an unconscious reflex (e.g., postural muscles or the diaphragm), skeletal muscles react to conscious control. Smooth muscle, or involuntary muscle, is found within the walls of organs and structures such as the esophagus, stomach, intestines, uterus, urethra, and blood vessels.

Skeletal muscle is further divided into two broad types: Type I (or “slow twitch”) and Type II (or “fast twitch”). Type I muscle fibers are dense with capillaries and are rich in mitochondria and myoglobin, which gives Type I muscle tissue a characteristic red color. In some cases, Type I muscle fibers carry more oxygen and sustain aerobic activity using fats or carbohydrates for fuel. Type I muscle fibers contract for long periods of time but with little force. Type II muscle fibers are further subdivided into three major subtypes (IIa, Ix, and IIb) that vary in both contractile speed and force generated. Type II muscle fibers contract quickly and powerfully but fatigue very rapidly, and therefore produce only short, anaerobic bursts of activity before muscle contraction becomes painful.

Unlike skeletal muscle, smooth muscle is not under conscious control.

Cardiac muscle is also an involuntary muscle but more closely resembles skeletal muscle in structure and is found only in the heart. Cardiac and skeletal muscles are striated in that they contain sarcomeres that are packed into highly regular arrangements of bundles. By contrast, the myofibrils of smooth muscle cells are not arranged in sarcomeres and therefore are not striated.

Muscle cells encompass any cells that contribute to muscle tissue. Exemplary muscle cells include myoblasts, satellite cells, myotubes, and myofibril tissues.

As used here, muscle force is proportional to the cross-sectional area (CSA), and muscle velocity is proportional to muscle fiber length. Thus, comparing the cross-sectional areas and muscle fibers between various kinds of muscles is capable of providing an indication of muscle atrophy. Various methods are known in the art to measure muscle strength and muscle weight, see, for example, “Musculoskeletal assessment: Joint range of motion and manual muscle strength” by Hazel M. Clarkson, published by Lippincott Williams & Wilkins, 2000. The production of tomographic images from selected muscle tissues by computed axial tomography and sonographic evaluation are additional methods of measuring muscle mass.

The term “antibody oligonucleotide conjugate (AOC)” refers to an antibody conjugated to a nucleotide.

The term “siRNA conjugate” or “siRNA-antibody conjugate” refers to an antibody conjugated to a siRNA.

The term “PRKAG2 siRNA-conjugate” or “PRKAG2 siRNA-antibody conjugate” refers to an antibody conjugated to an siRNA that is capable of hybridizing to a target sequence of the human PRKAG2 mRNA.

The term “PRKAG2-AOC” refers to an antibody conjugated to an oligonucleotide (e.g., siRNA) that is capable of hybridizing to a target sequence of the human PRKAG2 mRNA.

The term “PRKAG2 cardiac syndrome” is an autosomal dominant metabolic heart disease characterized by left ventricular hypertrophy (LVH), progressive conduction abnormalities, and ventricular pre-excitation.

The term “glycogen storage disease” refers to a disease caused by excessive cellular glucose uptake and pathological glycogen storage cells.

The term “antibody” is used in the broadest sense and covers fully assembled antibodies, antibody fragments that can bind antigen (e.g., Fab, F(ab′)₂, Fv, single chain antibodies, VHH, diabodies, antibody chimeras, hybrid antibodies, bispecific antibodies, humanized antibodies, and the like), and recombinant peptides comprising the foregoing.

The terms “antigen-binding fragments” and “antibody fragments” are used interchangeably to refer to a portion of an intact antibody, preferably the antigen-binding or variable region of the intact antibody. Examples of antibody fragments include Fab, F(ab′)₂, and Fv fragments; VHH; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

Depending on the amino acid sequence of the constant domain of their heavy chains, immunoglobulins can be assigned to different classes. There are five major classes of human immunoglobulins: IgA, IgD, IgE, IgG, IgM, and IgY; and several of these may be further divided into subclasses (isotypes), e.g., IgG1, IgG2, IgG3, IgG4, IgAQ1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, the CDRs of an antibody is determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J Mol. Biol., 196:901-917; Al-Lazikani et al., 1997, J. Mol. Biol., 273:927-948; Chothia et al., 1992, J. Mol. Biol., 227:799-817; Tramontano A et al., 1990, J Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al., 1999, Nucleic Acids Res., 27:209-212 (“IMGT CDRs”); or (iv) MacCallum et al., 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

The term “chimeric” antibody refers to an antibody in which a portion of the heavy and/or light chain is derived from a particular source or species, while the remainder of the heavy and/or light chain is derived from a different source or species.

The term “humanized antibody” refers to antibodies in which the framework or the CDRs have been modified to comprise the CDR of an immunoglobulin of different specificity as compared to that of the parent immunoglobulin.

As used herein, the term “sense strand” can be interchangeably used with the term “passenger strand,” and the term “antisense strand” can be interchangeably used with the term “guide strand.”

As used herein, the term “comprising” and its derivatives are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

As used herein, the term “consisting of” and its derivatives are intended to be closed ended terms that specify the presence of stated features, elements, components, groups, integers, and/or steps, and also exclude the presence of other unstated features, elements, components, groups, integers and/or steps

As used herein, the term “consisting essentially of” is intended to specify the presence of the stated features, elements, components, groups, integers, and/or steps as well as those that do not materially affect the basic and novel characteristic(s) of features, elements, components, groups, integers, and/or steps

### EXAMPLES

These examples are provided for illustrative purposes only and not to limit the scope of the claims provided herein.

### Example 1: Identification of siRNAs for the Regulation of Mouse and Human/NHP PRKAG2 Transcript

Bioinformatic siRNA library design against mouse and human/NHP PRKAG2 transcript:

Sequences of all siRNAs that can bind to PRKAG2 mRNA, or a pre-determined region of the PRKAG2 mRNA are collected to generate a starting set of PRKAG2 siRNAs. From the starting set of PRKAG2 siRNAs, the first eliminating step comprises eliminating one or more PRKAG2 siRNAs that have a single nucleotide polymorphism (SNP) and/or MEF<−5. Then, the second eliminating step comprises eliminating PRKAG2 siRNAs with 0 and 1 mismatches (MM) in the human/mouse/NHP transcriptomes. Then, the third eliminating step comprises eliminating PRKAG2 siRNAs with 0 mismatches (MM) in the human/mouse/NHP intragenic regions. Then, the next step comprises carrying forward one or more PRKAG2 siRNAs with predicted viability values>60. The next eliminating step comprises eliminating one or more PRKAG2 siRNAs with a match to a seed region of known miRNAs 1-1000. Then, the eliminating step continues with eliminating PRKAG2 siRNAs molecule with % GC content values of 75% or higher. The above selection steps yield the final 102 candidates PRKAG2 siRNAs selected from a starting set of more than 2000 PRKAG2 siRNAs. Table 10 summarizes the bioinformatics screening that identified 102 siRNAs (92 were 21/23 bp and 10 were 19/21 bp) that bind specifically to human PRKAG2 (NM_016203.4) (SEQ ID NO: 237) and have 100% homology with mouse (NM_145401.2) (SEQ ID NO: 238) and NHP (XM_005551219.3) (SEQ ID NO: 239). Thymine (T) and Uracil (U) are interchangeably used in Table 10.

TABLE 10

| Target site on | | | Antisense Strand/ | | Sense strand/ | |
|---|---|---|---|---|---|---|
| transcript | | Region | Guide Strand | SEQ | Passenger Strand | SEQ |
| NM_016203.4 | Exon | Target | Sequence (5′-3′) | ID NO: | Sequence (5′-3′) | ID NO: |
| 1205_1225 | 5 | CDS | CCAGCAUGCCGGCUUCCGCGG | 1 | CCGCGGAAGCCGGCAUGCUGG | 103 |
| 1206_1226 | 5 | CDS | UCCAGCAUGCCGGCUUCCGCG | 2 | CGCGGAAGCCGGCAUGCUGGA | 104 |
| 1207_1227 | 5 | CDS | CUCCAGCAUGCCGGCUUCCGC | 3 | GCGGAAGCCGGCAUGCUGGAG | 105 |
| 1209_1229 | 5 | CDS | UUCUCCAGCAUGCCGGCUUCC | 4 | GGAAGCCGGCAUGCUGGAGAA | 106 |
| 1210_1230 | 5 | CDS | CUUCUCCAGCAUGCCGGCUUC | 5 | GAAGCCGGCAUGCUGGAGAAG | 107 |
| 1211_1231 | 5 | CDS | GCUUCUCCAGCAUGCCGGCUU | 6 | AAGCCGGCAUGCUGGAGAAGC | 108 |
| 1212_1232 | 5 | CDS | AGCUUCUCCAGCAUGCCGGCU | 7 | AGCCGGCAUGCUGGAGAAGCU | 109 |
| 1213_1233 | 5 | CDS | CAGCUUCUCCAGCAUGCCGGC | 8 | GCCGGCAUGCUGGAGAAGCUG | 110 |
| 1279_1299 | 6 | CDS | GUGUGACCUCAUGAAUCGCAU | 9 | AUGCGAUUCAUGAGGUCACAC | 111 |
| 1280_1300 | 6 | CDS | UGUGUGACCUCAUGAAUCGCA | 10 | UGCGAUUCAUGAGGUCACACA | 112 |
| 1281_1301 | 6 | CDS | UUGUGUGACCUCAUGAAUCGC | 11 | GCGAUUCAUGAGGUCACACAA | 113 |
| 1282_1302 | 6 | CDS | CUUGUGUGACCUCAUGAAUCG | 12 | CGAUUCAUGAGGUCACACAAG | 114 |
| 1283_1303 | 6 | CDS | ACUUGUGUGACCUCAUGAAUC | 13 | GAUUCAUGAGGUCACACAAGU | 115 |
| 1284_1304 | 6 | CDS | CACUUGUGUGACCUCAUGAAU | 14 | AUUCAUGAGGUCACACAAGUG | 116 |
| 1285_1305 | 6 | CDS | ACACUUGUGUGACCUCAUGAA | 15 | UUCAUGAGGUCACACAAGUGU | 117 |
| 1287_1307 | 6 | CDS | UAACACUUGUGUGACCUCAUG | 16 | CAUGAGGUCACACAAGUGUUA | 118 |
| 1289_1309 | 6 | CDS | CAUAACACUUGUGUGACCUCA | 17 | UGAGGUCACACAAGUGUUAUG | 119 |
| 1290_1310 | 6 | CDS | UCAUAACACUUGUGUGACCUC | 18 | GAGGUCACACAAGUGUUAUGA | 120 |
| 1293_1313 | 6 | CDS | AUGUCAUAACACUUGUGUGAC | 19 | GUCACACAAGUGUUAUGACAU | 121 |
| 1296_1316 | 6 | CDS | ACGAUGUCAUAACACUUGUGU | 20 | ACACAAGUGUUAUGACAUCGU | 122 |
| 1297_1317 | 6 | CDS | AACGAUGUCAUAACACUUGUG | 21 | CACAAGUGUUAUGACAUCGUU | 123 |
| 1298_1318 | 6 | CDS | GAACGAUGUCAUAACACUUGU | 22 | ACAAGUGUUAUGACAUCGUUC | 124 |
| 1300_1320 | 6 | CDS | UGGAACGAUGUCAUAACACUU | 23 | AAGUGUUAUGACAUCGUUCCA | 125 |
| 1301_1321 | 6 | CDS | UUGGAACGAUGUCAUAACACU | 24 | AGUGUUAUGACAUCGUUCCAA | 126 |
| 1302_1322 | 6 | CDS | GUUGGAACGAUGUCAUAACAC | 25 | GUGUUAUGACAUCGUUCCAAC | 127 |
| 1303_1323 | 6 | CDS | GGUUGGAACGAUGUCAUAACA | 26 | UGUUAUGACAUCGUUCCAACC | 128 |
| 1304_1324 | 6 | CDS | UGGUUGGAACGAUGUCAUAAC | 27 | GUUAUGACAUCGUUCCAACCA | 129 |
| 1305_1325 | 6 | CDS | CUGGUUGGAACGAUGUCAUAA | 28 | UUAUGACAUCGUUCCAACCAG | 130 |
| 1306_1326 | 6 | CDS | ACUGGUUGGAACGAUGUCAUA | 29 | UAUGACAUCGUUCCAACCAGU | 131 |
| 1307_1327 | 6 | CDS | AACUGGUUGGAACGAUGUCAU | 30 | AUGACAUCGUUCCAACCAGUU | 132 |
| 1308_1328 | 6 | CDS | GAACUGGUUGGAACGAUGUCA | 31 | UGACAUCGUUCCAACCAGUUC | 133 |
| 1309_1329 | 6 | CDS | UGAACUGGUUGGAACGAUGUC | 32 | GACAUCGUUCCAACCAGUUCA | 134 |
| 1310_1330 | 6 | CDS | UUGAACUGGUUGGAACGAUGU | 33 | ACAUCGUUCCAACCAGUUCAA | 135 |
| 1311_1331 | 6 | CDS | UUUGAACUGGUUGGAACGAUG | 34 | CAUCGUUCCAACCAGUUCAAA | 136 |
| 1312_1332 | 6 | CDS | CUUUGAACUGGUUGGAACGAU | 35 | AUCGUUCCAACCAGUUCAAAG | 137 |
| 1313_1333 | 6 | CDS | GCUUUGAACUGGUUGGAACGA | 36 | UCGUUCCAACCAGUUCAAAGC | 138 |
| 1314_1334 | 6 | CDS | AGCUUUGAACUGGUUGGAACG | 37 | CGUUCCAACCAGUUCAAAGCU | 139 |
| 1316_1336 | 6 | CDS | CAAGCUUUGAACUGGUUGGAA | 38 | UUCCAACCAGUUCAAAGCUUG | 140 |
| 1317_1337 | 6 | CDS | ACAAGCUUUGAACUGGUUGGA | 39 | UCCAACCAGUUCAAAGCUUGU | 141 |
| 1654_1674 | 11 | CDS | CCCACUGAUAGGGUCAAUAAC | 40 | GUUAUUGACCCUAUCAGUGGG | 142 |
| 1655_1675 | 11 | CDS | UCCCACUGAUAGGGUCAAUAA | 41 | UUAUUGACCCUAUCAGUGGGA | 143 |
| 1656_1676 | 11 | CDS | UUCCCACUGAUAGGGUCAAUA | 42 | UAUUGACCCUAUCAGUGGGAA | 144 |
| 1695_1715 | 11 | CDS | AACUUGAGGAUUCUUUUGUGG | 43 | CCACAAAAGAAUCCUCAAGUU | 145 |
| 1700_1720 | 11 | CDS | GGAGGAACUUGAGGAUUCUUU | 44 | AAAGAAUCCUCAAGUUCCUCC | 146 |
| 1709_1729 | 11-12 | CDS | UAAAAAGCUGGAGGAACUUGA | 45 | UCAAGUUCCUCCAGCUUUUUA | 147 |
| 1712_1732 | 11-12 | CDS | ACAUAAAAAGCUGGAGGAACU | 46 | AGUUCCUCCAGCUUUUUAUGU | 148 |
| 1713_1733 | 11-12 | CDS | GACAUAAAAAGCUGGAGGAAC | 47 | GUUCCUCCAGCUUUUUAUGUC | 149 |
| 1714_1734 | 11-12 | CDS | AGACAUAAAAAGCUGGAGGAA | 48 | UUCCUCCAGCUUUUUAUGUCU | 150 |
| 1741_1761 | 12 | CDS | CUUCAUGAAGGCAGGCUUUGG | 49 | CCAAAGCCUGCCUUCAUGAAG | 151 |
| 1750_1770 | 12 | CDS | CAGGUUCUGCUUCAUGAAGGC | 50 | GCCUUCAUGAAGCAGAACCUG | 152 |
| 1752_1772 | 12 | CDS | UCCAGGUUCUGCUUCAUGAAG | 51 | CUUCAUGAAGCAGAACCUGGA | 153 |
| 1753_1773 | 12 | CDS | AUCCAGGUUCUGCUUCAUGAA | 52 | UUCAUGAAGCAGAACCUGGAU | 154 |
| 1758_1778 | 12 | CDS | AGCUCAUCCAGGUUCUGCUUC | 53 | GAAGCAGAACCUGGAUGAGCU | 155 |
| 1759_1779 | 12 | CDS | AAGCUCAUCCAGGUUCUGCUU | 54 | AAGCAGAACCUGGAUGAGCUU | 156 |
| 1760_1780 | 12 | CDS | CAAGCUCAUCCAGGUUCUGCU | 55 | AGCAGAACCUGGAUGAGCUUG | 157 |
| 1761_1781 | 12 | CDS | CCAAGCUCAUCCAGGUUCUGC | 56 | GCAGAACCUGGAUGAGCUUGG | 158 |
| 1884_1904 | 12-13 | CDS | ACAACUUUUCCUGACUCAUCC | 57 | GGAUGAGUCAGGAAAAGUUGU | 159 |
| 1885_1905 | 12-13 | CDS | UACAACUUUUCCUGACUCAUC | 58 | GAUGAGUCAGGAAAAGUUGUA | 160 |
| 1886_1906 | 12-13 | CDS | CUACAACUUUUCCUGACUCAU | 59 | AUGAGUCAGGAAAAGUUGUAG | 161 |
| 1889_1909 | 12-13 | CDS | UAUCUACAACUUUUCCUGACU | 60 | AGUCAGGAAAAGUUGUAGAUA | 162 |
| 1897_1917 | 13 | CDS | GGAAUAAAUAUCUACAACUUU | 61 | AAAGUUGUAGAUAUUUAUUCC | 163 |
| 1898_1918 | 13 | CDS | UGGAAUAAAUAUCUACAACUU | 62 | AAGUUGUAGAUAUUUAUUCCA | 164 |
| 1899_1919 | 13 | CDS | UUGGAAUAAAUAUCUACAACU | 63 | AGUUGUAGAUAUUUAUUCCAA | 165 |
| 1921_1941 | 13-14 | CDS | AGCAAGAUUAAUUACAUCAAA | 64 | UUUGAUGUAAUUAAUCUUGCU | 166 |
| 1927_1947 | 13-14 | CDS | CUCAGCAGCAAGAUUAAUUAC | 65 | GUAAUUAAUCUUGCUGCUGAG | 167 |
| 1928_1948 | 13-14 | CDS | UCUCAGCAGCAAGAUUAAUUA | 66 | UAAUUAAUCUUGCUGCUGAGA | 168 |
| 1929_1949 | 13-14 | CDS | UUCUCAGCAGCAAGAUUAAUU | 67 | AAUUAAUCUUGCUGCUGAGAA | 169 |
| 1931_1951 | 13-14 | CDS | UUUUCUCAGCAGCAAGAUUAA | 68 | UUAAUCUUGCUGCUGAGAAAA | 170 |
| 2148_2168 | 15 | CDS | GGUGUGAGGAUCAGGGCUUGC | 69 | GCAAGCCCUGAUCCUCACACC | 171 |
| 2149_2169 | 15 | CDS | UGGUGUGAGGAUCAGGGCUUG | 70 | CAAGCCCUGAUCCUCACACCA | 172 |
| 2155_2175 | 15-16 | CDS | ACCUGCUGGUGUGAGGAUCAG | 71 | CUGAUCCUCACACCAGCAGGU | 173 |
| 2203_2223 | 16 | 3′UTR-CDS | CGUCUACAUUCACGGCGGUCA | 72 | UGACCGCCGUGAAUGUAGACG | 174 |
| 2204_2224 | 16 | 3′UTR-CDS | GCGUCUACAUUCACGGCGGUC | 73 | GACCGCCGUGAAUGUAGACGC | 175 |
| 2205_2225 | 16 | 3′UTR-CDS | GGCGUCUACAUUCACGGCGGU | 74 | ACCGCCGUGAAUGUAGACGCC | 176 |
| 2206_2226 | 16 | 3′UTR | GGGCGUCUACAUUCACGGCGG | 75 | CCGCCGUGAAUGUAGACGCCC | 177 |
| 2267_2287 | 16 | 3′UTR | UUGCAGCCAGUGUUCAUGAGG | 76 | CCUCAUGAACACUGGCUGCAA | 178 |
| 2367_2387 | 16 | 3′UTR | CUGAAUCUUCAAGCACAUAAA | 77 | UUUAUGUGCUUGAAGAUUCAG | 179 |
| 2368_2388 | 16 | 3′UTR | CCUGAAUCUUCAAGCACAUAA | 78 | UUAUGUGCUUGAAGAUUCAGG | 180 |
| 2369_2389 | 16 | 3′UTR | GCCUGAAUCUUCAAGCACAUA | 79 | UAUGUGCUUGAAGAUUCAGGC | 181 |
| 2370_2390 | 16 | 3′UTR | AGCCUGAAUCUUCAAGCACAU | 80 | AUGUGCUUGAAGAUUCAGGCU | 182 |
| 2434_2454 | 16 | 3′UTR | ACUUUAAUGACAUACAGCAUU | 81 | AAUGCUGUAUGUCAUUAAAGU | 183 |
| 2436_2456 | 16 | 3′UTR | GCACUUUAAUGACAUACAGCA | 82 | UGCUGUAUGUCAUUAAAGUGC | 184 |
| 2440_2460 | 16 | 3′UTR | CAGUGCACUUUAAUGACAUAC | 83 | GUAUGUCAUUAAAGUGCACUG | 185 |
| 2441_2461 | 16 | 3′UTR | ACAGUGCACUUUAAUGACAUA | 84 | UAUGUCAUUAAAGUGCACUGU | 186 |
| 2442_2462 | 16 | 3′UTR | CACAGUGCACUUUAAUGACAU | 85 | AUGUCAUUAAAGUGCACUGUG | 187 |
| 2443_2463 | 16 | 3′UTR | ACACAGUGCACUUUAAUGACA | 86 | UGUCAUUAAAGUGCACUGUGU | 188 |
| 2444_2464 | 16 | 3′UTR | GACACAGUGCACUUUAAUGAC | 87 | GUCAUUAAAGUGCACUGUGUC | 189 |
| 2445_2465 | 16 | 3′UTR | GGACACAGUGCACUUUAAUGA | 88 | UCAUUAAAGUGCACUGUGUCC | 190 |
| 2446_2466 | 16 | 3′UTR | AGGACACAGUGCACUUUAAUG | 89 | CAUUAAAGUGCACUGUGUCCU | 191 |
| 2447_2467 | 16 | 3′UTR | CAGGACACAGUGCACUUUAAU | 90 | AUUAAAGUGCACUGUGUCCUG | 192 |
| 2448_2468 | 16 | 3′UTR | UCAGGACACAGUGCACUUUAA | 91 | UUAAAGUGCACUGUGUCCUGA | 193 |
| 3170_3190 | 16 | 3′UTR | AGUGUCAACAUUUUCAGAGCA | 92 | UGCUCUGAAAAUGUUGACACU | 194 |
| 1205_1223 | 5 | CDS | AGCAUGCCGGCUUCCGCGG | 93 | CCGCGGAAGCCGGCAUGCU | 195 |
| 1206_1224 | 5 | CDS | CAGCAUGCCGGCUUCCGCG | 94 | CGCGGAAGCCGGCAUGCUG | 196 |
| 1207_1225 | 5 | CDS | CCAGCAUGCCGGCUUCCGC | 95 | GCGGAAGCCGGCAUGCUGG | 197 |
| 1305_1323 | 6 | CDS | GGUUGGAACGAUGUCAUAA | 96 | UUAUGACAUCGUUCCAACC | 198 |
| 1307_1325 | 6 | CDS | CUGGUUGGAACGAUGUCAU | 97 | AUGACAUCGUUCCAACCAG | 199 |
| 1308_1326 | 6 | CDS | ACUGGUUGGAACGAUGUCA | 98 | UGACAUCGUUCCAACCAGU | 200 |
| 1309_1327 | 6 | CDS | AACUGGUUGGAACGAUGUC | 99 | GACAUCGUUCCAACCAGUU | 201 |
| 1310_1328 | 6 | CDS | GAACUGGUUGGAACGAUGU | 100 | ACAUCGUUCCAACCAGUUC | 202 |
| 2205_2223 | 16 | 3′UTR-CDS | CGUCUACAUUCACGGCGGU | 101 | ACCGCCGUGAAUGUAGACG | 203 |
| 2206_2224 | 16 | 3′UTR | GCGUCUACAUUCACGGCGG | 102 | CCGCCGUGAAUGUAGACGC | 204 |

### Example 2: Conjugate synthesis

siRNA Synthesis

siRNAs were synthesized following standard solid phase synthesis methods with a MerMadel2 synthesizer on standard 2′-O-methyl-base loaded 1000A CPG solid support. 2′-O-methyl, 2′-fluoro and 2′-O-C16 phosphonamidites were dissolved in anhydrous acetonitrile or anhydrous dichloromethane to make 0.1 M solution. 3% Trichloroacetic Acid in dichloromethane, 0.25M 5-ethylthio tetrazole in acetonitrile, 0.02 M iodine in pyridine/THF/H2O, 0.05 M (dimethylamino-methylidene)amino)-3H-1,2,4-dithiazoline-3-thione (DDTT) in pyridine/acetonitrile, acetic anhydride/THF/2,6-lutidine (Cap A) and 16% N-methylimidazole in THF (Cap B) were used as deblock, activation, oxidation, sulfurization and capping reagent, respectively. After synthesis, solid supports were dried and incubated in ammonium hydroxide/40% aqueous methylamine solution (AMA) for 2 hrs at room temperature. The CPG was filtered out and washed with water/methanol and the combined filtrate was concentrated under vacuum.

Single strands were purified by reverse-phase HPLC or anion exchange chromatography and desalted. Purified single strands were duplexed to make double stranded siRNA. siRNA passenger strands were synthesized with or without a C6-NH₂ conjugation handle at the 5′ end, attached through a phosphodiester or phosphorothioate linkage. siRNA guide strands were synthesized with or without a 5′-(E)-vinyl phosphonate modified nucleotide. Both single strands were identified by mass spectroscopy and their purity was assessed by HPLC analysis. Size exclusion chromatography (SEC) was used for duplex analysis.

Anti-human transferrin receptor antibody

Anti-human transferrin receptor antibody is a human IgG1 monoclonal antibody that binds to the human transferrin receptor 1. The antibody was produced as described in U.S. Pat. No. 10,913,800.

### Anti-Mouse Transferrin Receptor Antibody

Anti-mouse transferrin receptor antibody (CD71 mAb) is a rat IgG2a subclass monoclonal antibody that binds mouse CD71 or mouse transferrin receptor 1 (mTfR1). The antibody was produced by BioXcell and it is commercially available (Catalog #BE0175). IgG2a isotype control antibody

Rat IgG2a isotype control antibody was purchased from BioXcell (Clone 2A3, Catalog #BE0089). This antibody is specific to trinitrophenol and does not have any known antigens in mouse.

### AOC Synthesis

Antibody cys-MCC-siRNA conjugation by random cysteine conjugation

iRNAs were synthesized on a solid support made of controlled pore glass (CPG) employing the conventional phosphoramidite oligomerization chemistry and purified by high-performance liquid chromatography (HPLC). The passenger strand of the siRNA used for conjugation was synthesized with a C6 amino linker at the 5′-end. Functionalized siRNA (maleimide Linker-siRNA) was obtained by the reaction of linker with siRNA in slightly basic conditions (pH 7.4) in 50% DMSO for 30 min at room temperature, and reaction completion was confirmed by MS. Excess linker and DMSO were removed by spin filtration using AMICON 3K MWCO filters or TFF and buffer exchange with pH 6 sodium acetate buffer. PRKAG2 AOC was generated using a standard random cysteine conjugation method of murine anti-TfR1 monoclonal antibody and PRKAG2-targeting siRNA. The interchain disulfides of the antibody were partially and mildly reduced with TCEP tris(2-carboxyethyl)phosphine) to reveal cysteines that were highly and selectively reactive with the maleimide linker on the siRNA. This reaction formed a covalent link between the two advanced intermediates. The remaining thiols were capped with N-ethylmaleimide (NEM) prior to purification.

The reaction mixture was purified using strong anion exchange chromatography (SAX) to separate unreacted Ab, a drug-antibody ratio (DAR) species (i.e., one siRNA conjugated to one antibody is referred as DAR1), and excess siRNA. DAR1 AOC and DAR2 AOC fractions were collected, concentrated, buffer exchanged into PBS, and sterile filtered using a 0.2 μm filter.

Step 1: Antibody Interchain Disulfide Reduction with TCEP and Conjugation

The reduced antibody was made by adding 2 mM EDTA and 4 equivalents of TCEP in PBS to a solution of antibody in PBS or 20 mM Histidine, 145 mM NaCl, and pH 6 buffer. This solution was incubated for 4 hours at 37° C. To the reduced antibody solution, 1.1 eq of MCC-C6-siRNA was added. Analysis of the reaction mixture by analytical SAX column chromatography showed antibody-siRNA conjugate along with unreacted antibody and siRNA.

### Step 2: Purification

The crude reaction mixture was purified by AKTA Pure FPLC using strong anion exchange chromatography method-1. Fractions containing DAR1 and DAR>2 antibody-siRNA conjugates were separated, concentrated, buffer exchanged with pH 7.2 PBS, and sterile filtered using 0.2 μm filters.

### Step 3: Analysis of the Purified Conjugate

The isolated conjugates were characterized by SEC, SAX chromatography. The purity of the conjugate was assessed by analytical HPLC using either anion exchange chromatography method-2 or anion exchange chromatography method-3 as described herein. Isolated DAR1 conjugates are typically eluted at 9.0±0.3 min on analytical SAX method and are greater than 90% pure. The typical DAR>2 cysteine conjugate contains more than 90% DAR2 and less than 10% DAR3.

Fab′ Generation from mAb and Conjugation to siRNA

The antibody was buffer exchanged with pH 4.0, 20 mM sodium acetate/acetic acid buffer and made up to 5 mg/ml concentration. Immobilized pepsin (Thermo Scientific, Prod #20343) was added and incubated for 3 hours at 37° C. The reaction mixture was filtered using 30 kDa MWCO Amicon spin filters and pH 7.4 PBS. The retentate was collected and purified using size exclusion chromatography to isolate F(ab′)₂. The collected F(ab′)2 was then reduced by 10 equivalents of TCEP and conjugated with SMCC-C6-siRNA-PEG5 at room temperature in pH 7.4 PBS. Analysis of reaction mixture on SAX chromatography showed Fab′-siRNA conjugate along with unreacted Fab′ and siRNA-PEG.

### Fab-Oligonucleotide Conjugate (FabOC) Synthesis Method

### Step 1: Fab Expression

Antibody fragments used in FabOC were synthesized in-house using CHO or HEK293 expression and purified by protein L.

The sequences of the antibody fragments of human a-TfR1 antibody used in FabOC are provided in Table 11.

TABLE 11

| Compound | SEQ ID NO: | Sequence |
|---|---|---|
| FabOC | 205 | MEWSWVFLFFLSVTTGVHSQVQLVQSGAEVKKPGASVKVS |
| peptide | | CKASGYTFTNYWMHWVRQAPGQGLEWMGEINPINGRSNY |
| | | AQKFQGRVTLTVDTSISTAYMELSRLRSDDTAVYYCARGTR |
| | | AMHYWGQGTLVTVSSASTKGPSVFPLAPSSKSTSGGTAALG |
| | | CLVKDYFPEPVTVSWNSGALTSGVHTFPAVLQSSGLYSLSSV |
| | | VTVPSSSLGTQTYICNVNHKPSNTKVDKRVEPKSCDKTHTCC |
| FabOC | 206 | ATGGAATGGTCATGGGTTTTTTTGTTTTTCCTCTCAGTTAC |
| nucleotide | | GACTGGTGTCCATAGCCAAGTCCAACTGGTGCAGTCCGGT |
| | | GCGGAGGTTAAGAAGCCCGGAGCGAGCGTAAAGGTGAGT |
| | | TGTAAAGCGAGTGGATACACGTTCACGAACTATTGGATGC |
| | | ATTGGGTTCGACAAGCACCGGGTCAGGGACTTGAGTGGA |
| | | TGGGAGAAATTAATCCGATTAACGGTCGCAGTAACTATGC |
| | | GCAGAAATTCCAAGGCCGAGTAACTCTCACCGTGGACAC |
| | | GTCCATCTCTACAGCGTACATGGAACTCAGCAGGTTGCGC |
| | | TCTGACGATACCGCAGTTTATTATTGCGCGCGAGGGACGC |
| | | GGGCTATGCACTATTGGGGGCAGGGCACCCTCGTCACCGT |
| | | ATCATCTGCGAGTACGAAGGGACCTTCTGTGTTCCCATTG |
| | | GCTCCCAGCAGCAAAAGTACCAGTGGTGGAACAGCTGCG |
| | | CTTGGATGCCTGGTGAAAGATTATTTCCCCGAGCCGGTGA |
| | | CAGTCAGCTGGAACAGCGGCGCACTCACCAGCGGTGTAC |
| | | ATACGTTCCCGGCGGTTTTGCAATCTAGTGGCCTCTATTCC |
| | | CTTAGTTCCGTAGTTACCGTCCCATCTTCAAGCCTCGGAA |
| | | CCCAGACTTACATCTGCAACGTCAATCATAAGCCCAGTAA |
| | | CACAAAAGTTGATAAGAGAGTAGAGCCGAAATCCTGTGA |
| | | TAAGACCCACACATGTGGG |
| Light chain | 207 | MSVPTQVLGLLLLWLTDARCDIQMTQSPSSLSASVGDRVTIT |
| peptide | | CRTSENIYNNLAWYQQKPGKAPKLLIYAATNLAEGVPSRFS |
| | | GSGSGTDYTLTISSLQPEDFATYYCQHFWGTPLTFGGGTKVE |
| | | IKRTVAAPSVFIFPPSDEQLKSGTASVVCLLNNFYPREAKVQ |
| | | WKVDNALQSGNSQESVTEQDSKDSTYSLSSTLTLSKADYEK |
| | | HKVYACEVTHQGLSSPVTKSFNRGEC |
| Light chain | 208 | GCCTCCGGACTCTAGAGCCGCCACCATGAGCGTACCAAC |
| nucleotide | | CCAGGTGCTCGGACTCCTGTTGTTGTGGCTCACCGATGCA |
| | | AGATGCGATATACAAATGACACAAAGCCCAAGTAGTTTG |
| | | TCAGCCAGCGTAGGGGATAGAGTTACTATAACTTGCCGA |
| | | ACGTCTGAAAATATATATAATAACCTCGCGTGGTACCAGC |
| | | AGAAGCCCGGCAAGGCCCCTAAACTCCTCATTTATGCAGC |
| | | TACTAACCTCGCTGAAGGAGTACCATCAAGGTTCTCAGGC |
| | | AGCGGGTCTGGAACTGACTACACATTGACTATTTCAAGCC |
| | | TTCAGCCAGAGGACTTCGCTACATACTACTGTCAACACTT |
| | | CTGGGGGACTCCGCTTACTTTCGGAGGCGGTACCAAAGTG |
| | | GAGATAAAACGGACGGTTGCTGCTCCGAGCGTTTTTATAT |
| | | TCCCGCCCTCTGATGAACAGCTGAAATCAGGCACTGCGAG |
| | | CGTTGTTTGCTTGCTGAATAACTTTTACCCCCGCGAGGCG |
| | | AAAGTACAATGGAAGGTAGACAACGCACTGCAATCTGGG |
| | | AATAGTCAAGAGAGTGTTACCGAACAAGATTCAAAAGAT |
| | | TCCACTTATTCCCTTAGTTCTACTTTGACACTGAGCAAAGC |
| | | AGATTACGAGAAACATAAGGTCTACGCCTGCGAGGTGAC |
| | | GCACCAGGGCCTGAGCAGCCCAGTTACAAAGTCCTTCAAT |
| | | CGAGGTGAGTGTTAGGCGGCCGCTATAAGGGT |

### Step 2: Fab Conjugation to Oligonucleotide

To the purified Fab, 2 mM of FEDTA and 1 eq of TCEP in PBS was added. The solution was incubated at 37° C. for 4 hrs. Lyophilized siRNA was reconstituted in 50 mM PBS, pH 7.6 and pH adjusted to pH 7.2 followed by the addition of 8 equivalents of SMCC in DMS0. The reaction mixture was incubated at room temperature (RT) for 40 minutes. The SMCC activation was verified by mass spectrometry. The reaction mixture was treated and buffer exchanged with 10 mM Sodium Acetate buffer, pH 6.0 using 3K MWCO spin filters.

To the reduced Fab, 1 equivalent of SMCC activated siRNA solution was added and the reaction mixture was analyzed by analytical SAX.

### Purification of FabOC

The crude reaction mixture was purified by AKTA explorer pure FPLC using anion exchange chromatography method-1. Fractions containing FabOC (DAR1 and DAR2 Fab-siRNA conjugates) were separated, concentrated and buffer exchanged with pH 7.4 PBS.

### Step 3: Analysis of the Purified FabOC

The characterization and purity of the isolated conjugate was assessed by analytical HPLC using anion exchange chromatography method-2 or 3 as well as by SEC method-1. Purification and analytical Methods Anion exchange chromatography method (SAX)-1
Column: Tosoh Bioscience, TSKGel SuperQ-5PW, 21.5 mm ID X 15 cm, 13 μm
Solvent A: 20 mM TRIS buffer, pH 8.0; Solvent B: 20 mM TRIS, 1.5 M NaCl, pH 8.0;
Flow Rate: 6.0 ml/min

Gradient:

| % A | % B | Column Volume |
|---|---|---|
| 100 | 0 | 1.00 |
| 60 | 40 | 18.00 |
| 40 | 60 | 2.00 |
| 40 | 60 | 5.00 |
| 0 | 100 | 2.00 |
| 100 | 0 | 2.00 |

### Anion Exchange Chromatography (SAX) Method-2

Column: Thermo Scientific, ProPac™ SAX-10, Bio LC™, 4×250 mm
Solvent A: 80% 10 mM TRIS pH 8, 20% ethanol; Solvent B: 80% 10 mM TRIS pH 8, 20% ethanol, 1.5 M NaCl; Flow Rate: 0.75 ml/min

Gradient:

| Time | % A | % B |
|---|---|---|
| 0.0 | 90 | 10 |
| 3.00 | 90 | 10 |
| 11.00 | 40 | 60 |
| 13.00 | 40 | 60 |
| 15.00 | 90 | 10 |
| 20.00 | 90 | 10 |

### Anion Exchange Chromatography (SAX) Method-3

Column: Thermo Scientific, ProPac™ SAX-10, Bio LC™, 4×250 mm
Solvent A: 80% 10 mM TRIS pH 8, 20% ethanol; Solvent B: 80% 10 mM TRIS pH 8, 20% ethanol, 1.5 M NaCl
Flow Rate: 0.75 ml/min

Gradient:

| Time | % A | % B |
|---|---|---|
| 0.0 | 90 | 10 |
| 3.00 | 90 | 10 |
| 11.00 | 40 | 60 |
| 23.00 | 40 | 60 |
| 25.00 | 90 | 10 |
| 30.00 | 90 | 10 |

### Size Exclusion Chromatography (SEC) Method-1

Column: TOSOH Biosciences, TSKgelG3000SW XL, 7.8×300 mm, 5 μM
Mobile phase: 150 mM phosphate buffer
Flow Rate: 1.0 ml/min for 15 mins

### Example 3: Evaluation of selected PRKAG2 siRNAs-AOCs in WT mice

### Materials and Methods

PRKAG2 siRNAs-AOCs

The synthesis and purification of the PRKAG2 siRNAs-AOCs are described in Example 2.

### Animals

All animal studies were conducted following protocols in accordance with the Institutional Animal Care and Use Committee (IACUC) at Explora BioLabs, which adhere to the regulations outlined in the USDA Animal Welfare Act as well as the “Guide for the Care and Use of Laboratory Animals” (National Research Council publication, 8th Ed., revised in 2011). Mice were obtained from Charles River Laboratories or from Jackson Laboratories. Wild type C57BL/6J or 57BL6NCrl mice (8-10 week old) were dosed via intravenous (iv) infusion with the indicated AOCs and doses. qPCR mRNA levels evaluation in in vivo mouse

At scheduled take down day, hearts and gastrocnemius muscles were harvested and snap-frozen in liquid nitrogen. mRNA expression levels in target tissue were determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

Chemical Modification of siRNA

Top 6 performing siRNAs identified from the concentration response in in vitro, were synthesized using 7f.8s modifications and with and without VpUq chemical modifications as shown in in Table 12. As used herein, 7f.8s modifications refer a modification pattern of an antisense strand (guide strand): nsNfsnnnNfnnnnnnnNfnNfnnnnnsnsn (XosXfsXoXoXoXfXoXoXoXoXoXoXoXfXoXfXoXoXoXoXosXosXo), and sense strand (passenger strand): nsnsnnnnnnNfNfNfnnnnnnnnsnsn (XosXosXoXoXoXoXoXoXfXfXfXoXoXoXoXoXoXoXosXosXo), where Xf or Nf refers to 2′-Fluoro modified nucleotide where X or N is any base, Fluorine at 2′ position on ribose; Xo or n refers to 2′-O-methyl modified nucleotide, where X or n is any base and methylated oxygen at 2′ position on ribose; s refers to phosphorothioate internucleotide linkage modification, phosphorothioate linkage between bases. In some instances, modified guide strand comprises U at the 5′ end (e.g., VpUq or U). In some instances, such U replaces or substitutes the 5′ end nucleotide(s) of the base sequence or modified sequence of the guide strand. In some instances, modified guide strand includes an overhang sequence of “UU” or “dTdT” or “TT” at the 3′ end.

TABLE 12

| | | SEQ | Base Antisense/ | SEQ | Modified Antisense/ | SEQ | Modified Sense/ |
|---|---|---|---|---|---|---|---|
| | Modification | ID | Guide Strand | ID | Guide Strand | ID | Passenger Strand |
| Compound | Pattern | NO: | Sequence (5′-3′) | NO: | Sequence (5′-3′) | NO: | Sequence (5′-3′) |
| Seq58 | 7f.8s | 58 | UACAACUUUUCCUGACUCAUC | 209 | UosAfsCoAoAoCfUoUoU | 221 | (NH2C6)GosAosUoGo |
| (Seq58 − Vp) | | | | | oUoCoCoUoGfAoCfUoCo | | AoGoUoCoAfGfGfAoA |
| | | | | | AoUoCosUosUo | | oAoAoGoUoUoGosUos |
| | | | | | | | Ao |
| Seq58 + Vp | 7f.8s + VpUq | 58 | UACAACUUUUCCUGACUCAUC | 210 | VpUqsAfsCoAoAoCfUoU | 222 | (NH2C6)GosAosUoGo |
| (Seq58 + VpUg) | | | | | oUoUoCoCoUoGfAoCfUo | | AoGoUoCoAfGfGfAoA |
| | | | | | CoAoUoCosUosUo | | oAoAoGoUoUoGosUos |
| | | | | | | | Ao |
| Seq61 | 7f.8s | 61 | GGAAUAAAUAUCUACAACUUU | 211 | UosGfsAoAoUoAfAoAoU | 223 | AosAosAoGoUoUoGoU |
| (Seq61 − Vp) | | | | | oAoUoCoUoAfCoAfAoCo | | oAfGfAfUoAoUoUoUo |
| | | | | | UoUoUosUosUo | | AoUoUosCosAo |
| Seq61 + Vp | 7f.8s + VpUq | 61 | GGAAUAAAUAUCUACAACUUU | 212 | VpUqsGfsAoAoUoAfAoA | 224 | AosAosAoGoUoUoGoU |
| (Seq61 + VpUq) | | | | | oUoAoUoCoUoAfCoAfAo | | oAfGfAfUoAoUoUoUo |
| | | | | | CoUoUoUosUosUo | | AoUoUosCosAo |
| Seq62 | 7f.8s | 62 | UGGAAUAAAUAUCUACAACUU | 213 | UosGfsGoAoAoUfAoAoA | 225 | AosAosGoUoUoGoUoA |
| (Seq62 − Vp) | | | | | oUoAoUoCoUfAoCfAoAo | | oGfAfUfAoUoUoUoAo |
| | | | | | CoUoUosUosUo | | UoUoCosCosAo |
| Seq62 + Vp | 7f.8s + VpUq | 62 | UGGAAUAAAUAUCUACAACUU | 214 | VpUqsGfsGoAoAoUfAoA | 226 | AosAosGoUoUoGoUoA |
| (Seq62 + VpUq) | | | | | oAoUoAoUoCoUfAoCfAo | | oGfAfUfAoUoUoUoAo |
| | | | | | AoCoUoUosUosUo | | UoUoCosCosAo |
| Seq64 | 7f.8s | 64 | AGCAAGAUUAAUUACAUCAAA | 215 | UosGfsCoAoAoGfAoUoU | 227 | (NH2C6)UosUosUoGo |
| (Seq64 − Vp) | | | | | oAoAoUoUoAfCoAfUoCo | | AoUoGoUoAfAfUfUoA |
| | | | | | AoAoAosUosUo | | oAoUoCoUoUoGosCos |
| | | | | | | | Ao |
| Seq64 + Vp | 7f.8s + VpUq | 64 | AGCAAGAUUAAUUACAUCAAA | 216 | VpUqsGfsCoAoAoGfAoU | 228 | UosUosUoGoAoUoGoU |
| (Seq64 + VpUg) | | | | | oUoAoAoUoUoAfCoAfUo | | oAfAfUfUoAoAoUoCo |
| | | | | | CoAoAoAosUosUo | | UoUoGosCosAo |
| Seq77 | 7f.8s | 77 | CUGAAUCUUCAAGCACAUAAA | 217 | UosUfsGoAoAoUfCoUoU | 229 | UosUosUoAoUoGoUoG |
| (Seq77 − Vp) | | | | | oCoAoAoGoCfAoCfAoUo | | oCfUfUfGoAoAoGoAo |
| | | | | | AoAoAosUosUo | | UoUoCosAosAo |
| Seq77 + Vp | 7f.8s + VpUq | 77 | CUGAAUCUUCAAGCACAUAAA | 218 | VpUqsUfsGoAoAoUfCoU | 230 | UosUosUoAoUoGoUoG |
| (Seq77 + VpUq) | | | | | oUoCoAoAoGoCfAoCfAo | | oCfUfUfGoAoAoGoAo |
| | | | | | UoAoAoAosUosUo | | UoUoCosAosAo |
| Seq98 | 7f.8s | 98 | ACUGGUUGGAACGAUGUCA | 219 | UosCfsUoGoGoUfUoGoG | 231 | UosGosAoCoAoUoCfG |
| (Seq98 − Vp) | | | | | oAoAoCoGoAfUoGfUoCo | | fUfUoCoCoAoAoCoCo |
| | | | | | AosUosUo | | AosGosAo |
| Seq98 + Vp | 7f.8s + VpUq | 98 | ACUGGUUGGAACGAUGUCA | 220 | VpUqsCfsUoGoGoUfUoG | 232 | UosGosAoCoAoUoCfG |
| (Seq98 + VpUq) | | | | | oGoAoAoCoGoAfUoGfUo | | fUfUoCoCoAoAoCoCo |
| | | | | | CoAosUosUo | | AosGosAo |

*Vp refers to 5′ Vinyl Phosphonate; Uq refers to UNA-U Unlocked nucleobase analog to uridine; Xf refers to 2′-fluoro\
modified nucleotide where X is any base; Xo refers to 2′-O-methyl modified nucleotide, where X is any base; s refers
to phosphorothioate internucleotide linkage; and (NH₂C₆) refers to aminohexyl handle/linker. In some instances, (NH₂C₆)
is coupled to the 5′ end of the passenger strand (sense strand).

siRNAs were conjugated to anti-transferrin receptor 1(TfR1) antibody using a SMCC linker, to produce AOCs and were tested in WT mice. 8 weeks old C57BL mice were intravenously administered with AOCs, at 3 mg/kg (siRNA dose). Heart and gastrocnemius tissues were collected at 28 and 56 days after AOC treatment. Control animals were injected with PBS.

### Results

Top 6 performing siRNAs identified from the concentration response in vitro, were synthesized using 7f.8s modifications and with and without VpUq chemical modifications (Table 12).

In the heart tissues, over 75% knockdown (KD) of PRKAG2 mRNA expression was achieved with Seq61+Vp at 28 days, and KD was maintained up to 56 days. In the gastrocnemius tissues, over 75% KD was achieved with Seq61+Vp at 28 days and maintained up to 56 days.

### Example 4: Dose Response and PK/PD Activity of PRKAG2-Targeting AOC in WT Mice

### Materials and Methods

A murine transferrin receptor 1 (TfR1) antibody was conjugated to siPRKAG2 (Seq61+VpUq) to generate siPRKAG2-AOC. siRNA sequence used in Example 4 is shown in Table 13. As used herein, 7f.8s modifications refer a modification pattern of an antisense strand (guide strand): nsNfsnnnNfnnnnnnnNfnNfnnnnnsnsn (XosXfsXoXoXoXfXoXoXoXoXoXoXoXfXoXfXoXoXoXoXosXosXo), and sense strand (passenger strand): nsnsnnnnnnNfNfNfnnnnnnnnsnsn (XosXosXoXoXoXoXoXoXfXfXfXoXoXoXoXoXoXoXosXosXo), where Xf or Nf refers to 2′-Fluoro modified nucleotide where X or N is any base; Xo or n refers to 2′-O-methyl modified nucleotide, where X or n is any base; s refers to phosphorothioate intemucleotide linkage. In some instances, modified guide strand comprises U at the 5′ end (e.g., VpUq or U). In some instances, such U replaces or substitutes the 5′ end nucleotide(s) of the base sequence or modified sequence of the guide strand. In some instances, modified guide strand includes an overhang sequence of “UU” or “dTdT” or “TT” at the 3′ end.

TABLE 13

| | | Base | | |
|---|---|---|---|---|
| | Modifi- | Antisense/Guide | Modified | Modified |
| | cation | Strand Sequence | Antisense/Guide Strand | Sense/Passenger Strand |
| Compound | Pattern | (5′-3′) | Sequence (5′-3′) | Sequence (5′-3′) |
| siPRKAG2 | 7f.8s | GGAAUAAAUAUC | VpUqsGfsAoAoUoAfAoA | AosAosAoGoUoUoGoUo |
| (Seq61 + VpUq) | + VpUq | UACAACUUU | oUoAoUoCoUoAfCoAfAo | AfGfAfUoAoUoUoUoAo |
| | | (SEQ ID NO: 61) | CoUoUoUosUosUo (SEQ | UoUosCosAo (SEQ ID |
| | | | ID NO: 212) | NO: 224) |

*Vp refers to 5′ Vinyl Phosphonate; Uq refers to UNA-U Unlocked nucleobase analog to uridine; Xf
refers to 2′-fluoro modified nucleotide where X is any base; Xo refers to 2′-O-methyl modified
nucleotide, where X is any base; s refers to phosphorothioate internucleotide linkage. In some
instances, (NH₂C₆) is coupled to the 5′ end of the passenger strand (sense
strand), and (NH₂C₆) refers to aminohexyl handle/linker.

### PRKAG2-Targeting AOC Synthesis

siRNAs were synthesized on a solid support made of controlled pore glass (CPG) employing the conventional phosphoramidite oligomerization chemistry. siPRKAG2-AOC (also referred as mTfR1-PRKAG2) was generated using a standard random cysteine conjugation method of murine anti-TfR1 monoclonal antibody and PRKAG2-targeting siRNA, siPRKAG2. In practice, the interchain disulfides of the antibody are partially and mildly reduced with TCEP tris(2-carboxyethyl)phosphine) to reveal cysteines that are highly and selectively reactive with the maleimide linker the siRNA. This reaction forms a covalent link between the two advanced intermediates. The reaction mixture was purified using strong anion exchange chromatography to ensure a drug-antibody ratio (DAR) equal to 1 (i.e., one siRNA molecule conjugated to one antibody is referred as DAR1). Collected AOC fractions were concentrated, buffer exchanged into PBS, and sterile filtered using a 0.2 μm filter.

### Animal Treatment and Tissue Collection

Mice used in this example were blinded for treatment group. WT mice (C57BL/6J) were administered with PBS or siPRKAG2-AOC once by a single intravenous (IV) bolus injection into the tail vein at 5 mL/kg body weight. Body weights were measured to within 0.1 grams. Injection into the tail vein was performed using 23-25 gauge needles. Body weights and dose volumes were recorded. Tissue necropsy samples were collected 28 days post dose. Mice were euthanized by C02 asphyxiation, and tissue collection was performed immediately after euthanasia. Skeletal muscles (gastrocnemius) and hearts were collected at necropsy, and sample weight (approximately 20-40 mg per tissue sample) was recorded. Tissues were collected into a Lysing Matrix D tube, frozen on dry ice and then stored in a freezer set to maintain −80° C., until processing.

siRNA Quantification in Tissue Samples

A specific stem-loop real-time quantitative polymerase chain reaction (SL-RT-qPCR) assay was designed to measure the guide strand of siPRKAG2 (Seq61+VpUq) in tissue homogenate. The SL-RT-qPCR assay along with the general primer and TaqMan probe have been previously described in Chen, 2005. Briefly, calibration standards for an 8-point standard curve were generated by a 1:10 serial dilution of 6 siRNA Sequences in TRIzol (Thermo Fisher). Study samples were diluted in assay diluent (TE Buffer with 0.1% Triton-X-100) up to the recommended Minimum Required Dilution (MRD; 1:100). The calibration standards were diluted in assay diluent. A siRNA-specific reverse transcription (RT) primer (custom from Integrated DNA Technologies) was added to the calibration standards and study samples, followed by an annealing and a reverse transcription step (Fisher Scientific, #43-665-97) on a thermocycler. The RT products were quantified via conventional TaqMan RT-PCR (Fisher Scientific, #44-445-58) which includes a siRNA-specific forward primer (custom from Integrated DNA Technologies), a universal reverse primer (custom from Integrated DNA Technologies), and a dye labeled siRNA-specific TaqMan probe (custom from Thermo Fisher). Raw Ct values were exported using the QuantStudio Real-Time PCR and Excel software. The standard curve of Ct values vs. log base 10 of corresponding siRNA concentrations was generated in GraphPad Prism 9.3.1. A simple linear regression of the siRNA standard curve was performed in GraphPad Prism, and the linear equation was used to interpolate study sample plasma concentrations in Excel. The lower limit of quantification was 5 μM. Sample values are reported as the average of technical replicates. Linear regressions of siRNA standard curves were performed in GraphPad Prism 10, and the linear equations were used to interpolate tissue and sample concentrations.

### RNA Isolation and Gene Expression Analysis in Tissue Samples

Tissue homogenate supernatants were used for RNA isolation using a Direct-zol-96 RNA isolation kit (Zymo Research, Cat #R2056) according to the manufacturer's instructions. RNA was treated with DNase and eluted into water following manufacturer's instructions. Between 100-500 ng of purified RNA was converted to cDNA using a High-Capacity cDNA Reverse Transcription Kit (Thermo Fisher, Cat #4368813) and a SimpliAmp Thermal Cycler (Applied Biosystems, Foster City, CA, USA). cDNA was analyzed by qPCR using TaqMan Fast Universal Master Mix II (Thermo Fisher) and TaqMan probes (Thermo Fisher) in duplicates, using QuantStudio 6 or 7 Flex Real-Time PCR instruments (Applied Biosystems). Data were analyzed by QuantStudio™ Real-Time PCR Software v1.3 (Applied Biosystems). The percentage of target gene expression was determined relative to the control treatment using GAPDH as reference gene, and relative to the control treatment using the 2\hat{ }(−ΔΔCt) method. TaqMan probes used were purchased at Thermo Fisher. Mouse Prkag2: Mm00513977_ml and Mouse Gapdh: Mm99999915_g1.

### Results

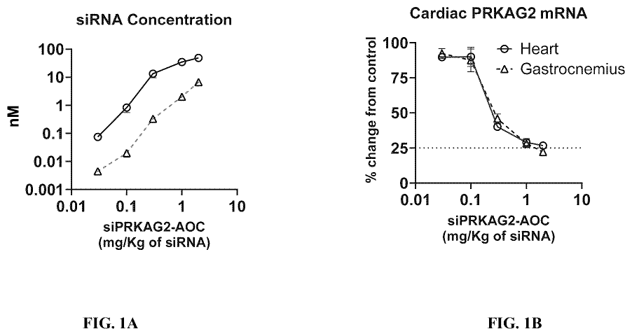

The PK/PD profile of siPRKAG2 in vivo was evaluated in WT mice at 28 days after the treatment. A dose response for the tissue concentrations (TC) for siPRKAG2-AOC was observed in heart and skeletal muscle at day 28 (FIGS. 1A-1B). A 2 mg/kg dose produced around 50 nM of TC in the hearts and about 10 nM of TC in the skeletal muscle. (FIG. 1A). siPRKAG2-AOC demonstrated a dose response for PRKAG2 mRNA reduction at 28 days after the treatment in the heart and in the gastrocnemius muscles (FIG. 1B). About 75% PRKAG2 mRNA reduction was observed in heart with 2 mg/kg dose and with 1 mg/kg dose (siRNA dose) at 28 days post dosing. 50% PRKAG2 mRNA reduction was observed in heart with 0.3 mg/kg dose. No substantial PRKAG2 mRNA reduction was observed with 0.1 mg/kg and 0.03 mg/kg dose.

These results indicate that the siPRKAG2 (Seq61+VpUq) demonstrated a robust, long-lasting activity in the cardiac and skeletal muscle after systemic administration.

### Example 5: In Vivo Efficacy of PRKAG2-Targeting AOC in WT and PRKAG2 Mutant Mice

### Materials and Methods

The materials and methods are described in Example 4. WT mice (C57BL/6J) and PRKAG2 mutant mice (TG) carrying a PRKAG2 point mutation R528G (corresponding to human R531G mutation) were used in this example.

TABLE A
Study Design

| | | | | Take | siRNA | AOC | Dose | Dosing |
|---|---|---|---|---|---|---|---|---|
| | No. of | | No of | Down | Conc. | Conc. | siRNA | Volume |
| Mice | Mice | Test compound | Doses | Day | (mg/ml) | (mg/ml) | (mg/kg) | (ml/kg) |
| WT | 11 | PBS | 2 | 168 | 0 | 0 | 0 | 5 |
| WT | 7 | siPRKAG2 | 2 | 168 | 0.6 | 6.4 | 3 | 5 |
| | | (Seq61 + VpUq) | | | | | | |
| TG | 6 | PBS | 2 | 168 | 0 | 0 | 0 | 5 |
| TG | 6 | siPRKAG2 | 2 | 168 | 0.6 | 6.4 | 3 | 5 |
| | | (Seq61 + VpUq) | | | | | | |

### Animal Treatment and Tissue Collection

Mice in the study were blinded for treatment group. WT mice were administered with PBS or siPRKAG2-AOC (as presented in Example 4) two times (ql2w) by intravenous (IV) bolus injection into the tail vein at 5 ml/kg body weight. Body weights were measured to within 0.1 grams. Injection into the tail vein was performed using 23-25 gauge needles. Body weights and dose volumes were recorded. Tissue necropsy samples were collected at 28 days and at 56 days post dose. Mice were euthanized by CO₂ asphyxiation. Tissue collection was performed immediately after euthanasia. Skeletal muscles (gastrocnemius) and hearts were collected at necropsy, and sample weight (approximately 20-40 mg per tissue sample) was recorded, collected into a Lysing Matrix D tube, frozen on dry ice and then stored in a freezer set to maintain −80° C., until processing.

### Glycogen Quantification in Tissue Samples

Glycogen accumulation was measured according to the protocol developed by Schaubroeck et al., 2022, with minor modifications, using ab65620 kit (Abcam), according to manufacturer's instructions. 20-30 mg of tissue were pulverized using a CP02 cryoPREP Automated Dry Pulverizer (Covaris). 0.5M NaOH was added and chilled samples were homogenized using a polytron homogenizer. Samples were boiled to 100° C. for 10 minutes, and allowed to cool down at R. T. for 10 minutes. 9.5% (w/v) Na₂SO₄ was added as co-precipitant agent. Then, 100% ethanol was added to precipitate the glycogen. Samples were centrifugated for 10 minutes at 2000 g. Supernatant was discarded and pellet was allowed to air dry for 5-10 minutes. Pellet was resuspended in 200-250 μl of DI water (Thermo Scientific, Ref A57775). Samples were stored in −80° C. until further analysis.

### Results

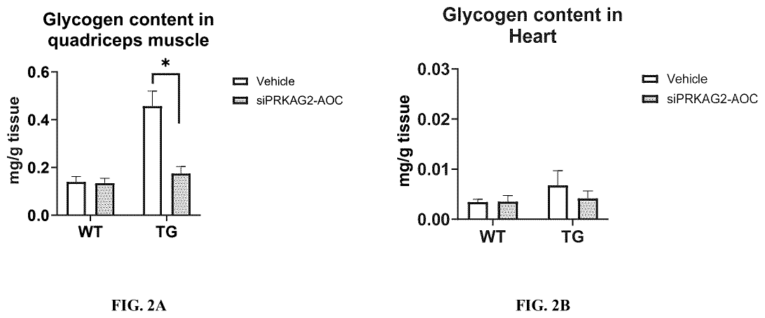

In WT mice, the glycogen content in the quadriceps muscle was not significantly changed after administration of siPRKAG2-AOC. However, in TG mice, the glycogen content in the quadriceps muscle was significantly decreased (by more than 50% reduction compared to PBS (vehicle) treated group) after administration of siPRKAG2-AOC (FIG. 2A). These results indicate that siPRKAG2-AOC can effectively reduce glycogen content in skeletal muscle in the PRKAG2 mutant mice.

The glycogen content in the heart muscle was also measured in the WT and TG mice (FIG. 2B). Compared to WT, glycogen content in the TG mice was increased with vehicle treatment. With siPRKAG2-AOC treatment, glycogen content in the heart muscle was decreased in TG mice.

Overall, these results demonstrate that siPRKAG2-AOC can prevent or reverse glycogen accumulation in the skeletal muscle and cardiac tissue in the PRKAG2 mutant mice.

### Example 6: In Vivo Evaluation of PRKAG2-Targeting AOC in WT and PRKAG2 Mutant Mice

### Materials and Methods

The materials and methods used in this example are described in Example 4. WT mice (C57BL/6J) and PRKAG2 mutant mice (TG) carrying a PRKAG2 point mutation R528G (corresponding to human R531G mutation) were used in this example.

TABLE B
Study Design

| | | | | | siRNA | AOC | Dose | Dosing |
|---|---|---|---|---|---|---|---|---|
| | No. of | | No of | Take | Conc. | Conc. | siRNA | Volume |
| Mice | Mice | Test Article | Doses | Down Day | (mg/ml) | (mg/ml) | (mg/kg) | (ml/kg) |
| WT | 6 | PBS | 3 | 224 | 0 | 0 | 0 | 5 |
| WT | 8 | siPRKAG2 | 3 | 224 | 0.6 | 6.4 | 3 | 5 |
| | | (Seq61 + VpUq) | | | | | | |
| TG | 9 | PBS | 3 | 224 | 0 | 0 | 0 | 5 |
| TG | 9 | siPRKAG2 | 3 | 224 | 0.6 | 6.4 | 3 | 5 |
| | | (Seq61 + VpUq) | | | | | | |

### Cardiac Echo Doppler of Heart Function in Anesthetized Mice

All animal procedures were performed in accordance with the IACUC protocols. Mice were kept under identical housing conditions (12 h light/dark cycle, standard diet ad libitum, 21° C. room temperature prior to echocardiographic assessment. Echo was performed using Vevo 3100 high-resolution Imaging System (Fujifilm). In order to separate early (E) and late (A) peak for doppler measurements, mice were put under anesthesia (5% isoflurane for induction and then maintain at 2-3% levels at 0.2 L/hr) to reduce heart rate to around 400 beat per minute. Animals were naired and restrained. A snout was placed in the nose cone and mice were taped to the platform using surgical tape. Electrode gel was applied to the contact areas between the paws and the electrode surface. Pre-warmed sonography gel was applied to the chest area. Probes were positioned pointing from apex to aorta to obtain the four-chamber view with the guidance from color doppler to identify transmitral flow measurements. The Doppler beam was then aligned parallel to the blood flow (red) to record maximal transmitral flow velocities. The early (E) and late (A) diastolic transmitral flow velocity peaks were measured from the Doppler waveform. The E/A ratio was calculated by dividing the E wave velocity by the A wave velocity. (Shumin Gao, 2011 and Maria Villalba-Orero, 2022).

### ECG Recordings

Surface Electrocardiography (ECG) signal (lead I and II via limb electrodes) was acquired with ADInstruments Bio Amps and Powerlab C.

### ECG Analysis

ECG data were analyzed using LabChart 8 pro. 5 to 10 minutes of ECG signal were recorded at a sampling rate of 4k/s and range of 2 mV. Mice were given 5% isoflurane for 30 seconds and then maintained under anesthesia with 2% isoflurane to keep HR around high 500s for the final analysis in a more physiological setting. The whole recorded ECG tracing was visually reviewed and average view of 100 consistent beats were manually edited for measurements to detect possible arrhythmias and aberrant ECG complexes. P amplitudes was defined using onset of the P wave as isoelectric line.

### Results

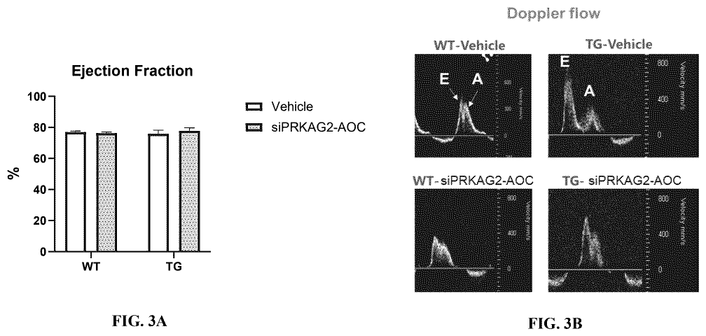

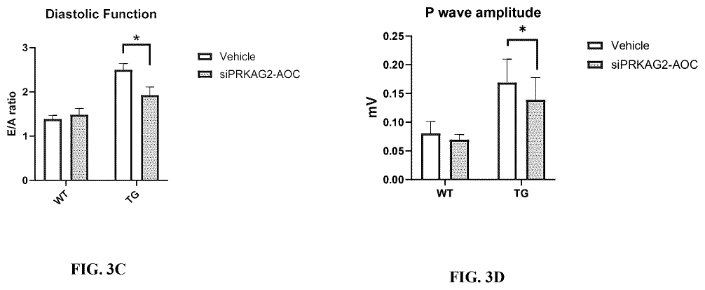

To assess cardiac function, echo-doppler was performed at 32 weeks. The PRKAG2 mutant mice (TG) showed the same ejection fraction of WT animals and no significant effect was noted with siPRKAG2-AOC injection (FIG. 3A). TG mice had a higher E/A ratio compared to WT mice (FIGS. 3B-3C). Representative images of the cardiac echo-doppler are shown in FIG. 3B. As shown in FIG. 3C, upon siPRKAG2-AOC treatment, the E/A ratio measured in the TG mice was significantly reduced and comparable to the E/A ratio measured in WT mice treated with PBS. In addition, as shown in FIG. 3D, upon siPRKAG2-AOC treatment, amplitude of P wave measured from the ECG was significantly decreased (* indicates p<0.05) in the TG mice, while the effect of the siPRKAG2-AOC treatment to the WT mice was not significant.

These results demonstrate that siPRKAG2-AOC demonstrated a robust activity and efficacy in improving diastolic function in PRKAG2 mutant mice.

### Example 7: PK/PD Activity of PRKAG2-Targeting AOC in Non-Human Primate (NHP) Study

Non-Human primate (NHP) study was conducted to investigate the pharmacokinetic (PK) and pharmacodynamic (PD) profiles of siPRKAG2-hAOC following a single intravenous (IV) injection to cynomolgus monkeys. The animal study was performed at Key Prime Research (KPR) in South Korea, following Institutional Animal Care and Use Committee (IACUC) protocols at KPR. Through the study, animals were housed individually in a stainless-steel wire cage (720W x 700 L x 800H mm). Animals had access to about 60 g of diet (Teklad Global Certified 20% Protein Primate Diet 2050C), twice daily. Animals had ad libitum access to filtered, ultraviolet light-irradiated municipal tap water at all times.

Pharmacokinetics (PK) and Pharmacodynamics (PD) profile of siPRKAG2-hAOC following a single intravenous (IV) administration was evaluated in wild type cynomolgus monkeys (naive). The siPRKAG2-hAOC used in this example was composed of human transferrin receptor 1 (TfR1) antibody conjugated to a small interfering ribonucleic acid (siRNA) targeting the human protein kinase AMP-activated non-catalytic subunit gamma 2 (PRKAG2) gene, which nucleic acid sequence is shown in Table 13.

Three males/group, approximately 2 to 4 years of age, and approximately 2 to 4 kg body weight at start of treatment received vehicle control (0 mg/kg) or siPRKAG2-hAOC at 3 mg/kg siRNA dose on Day 1. Punch biopsies of the gastrocnemius tissues were collected during acclimatation (predose, corresponding to day -6) and on Day 15 post-dosing. Heart and gastrocnemius tissues were collected at necropsy on Day 29. On Day 29, all animals were sacrificed.

Observations and measurements for mortality, clinical signs including detailed clinical sign and injection site observation, body weight, food consumption, electrocardiography (ECG), clinical and anatomic pathology were recorded. ECG was conducted on pre-dose and Day 28. Blood and tissue samples including skeletal muscles, heart, liver, and kidney for PK and/or PD analysis were collected and analyzed. No significant changes or findings were observed in mortality, clinical signs, body weight, food consumption, ECG, clinical and anatomic pathology in siPRKAG2-hAOC treated group compared to the vehicle control administered group.

### Materials and Methods

Tissue samples were evaluated for mRNA expression using a comparative RT-qPCR assay. Tissues samples ranging from 25-50 mg were homogenized in 1 mL of TRIzol (Thermo Fisher) on the OMNI Bead Ruptor Elite system (OMNI International). Total RNA was isolated from tissue homogenate supernatant using the Direct-zol-96 RNA kit (Zymo Research) according to the manufacturer's instructions. 250 ng of purified RNA was reverse transcribed to cDNA using the High-Capacity cDNA Reverse Transcription Kit (Applied Biosystem) and SimpliAmp Thermal Cycler (Applied Biosystem). The amount of cDNA used per qPCR reaction was 50 ng (for muscle analysis) or 100 ng (for non-muscle tissue analysis). qPCR was performed using TaqMan Fast Universal Master Mix II (Thermo Fisher) and commercially available Tagman Assays. Each sample was run in technical duplicates in a Quant Studio instrument (Thermo Fisher). qPCR data was analyzed using the ΔΔCt method with gene of interest normalized to Small RNA Binding Exonuclease Protection Factor La gene (SSB) expression. PRKAG2 expression in heart, liver and kidney was calculated using formula: 100*[2\hat{ }(ΔCt sample-Average ΔCt_{1M001_1M002})]. PRKAG2 expression in skeletal muscle was calculated using formula: 100*[2\hat{ }(ΔCt sample−ΔCt_{Predose})]; where Ct is the qPCR cycle threshold. Data was analyzed using GraphPad Prism 10 and is expressed as mean±SD.

Tissues samples ranging from 25-50 mg were homogenized in 1 mL of TRIzol (Thermo Fisher) on the OMNI Bead Ruptor Elite system (OMNI International). Total RNA was isolated from tissue homogenate supernatant using the Direct-zol-96 RNA kit (Zymo Research) according to the manufacturer's instructions. 250 ng of purified RNA was reverse transcribed to cDNA using the HighCapacity cDNA Reverse Transcription Kit (Applied Biosystem) and SimpliAmp Thermal Cycler (Applied Biosystem). The amount of cDNA used per qPCR reaction was 50 ng for muscle analysis. qPCR was performed using TaqMan Fast Universal Master Mix II (Thermo Fisher) and commercially available Tagman Assays (PRKAG2 Mf04185244_ml-FAM; SSB Mf04187362_g1-VIC). Each sample was run in technical duplicates in a Quant Studio instrument (Thermo Fisher). qPCR data was analyzed using the ΔΔCt method with gene of interest normalized to Small RNA Binding Exonuclease Protection Factor La gene (SSB) expression. PRKAG2 expression in heart was calculated using formula: 100*[2\hat{ }(ΔCt sample -Average ΔCt1M001, 1M002)]. PRKAG2 expression in skeletal muscle was calculated using formula: 100*[2\hat{ }(ΔCt sample -ΔCt Predose)]; where Ct is the qPCR cycle threshold. Data was analyzed using GraphPad Prism 10 and is expressed as mean±SD.

### PRKAG2 Protein Expression in NHP

Tissue samples were dipped in liquid nitrogen and pulverized. The sample was homogenized in Pierce RIPA Lysis and Extraction Buffer (Thermo Scientific, Cat #89901) containing one Pierce Protease Inhibitor mini table (Thermo Scientific, Cat #A32953), using a handheld polytron homogenizer. Samples were centrifuged and the supernatant was collected. Samples were diluted with 20% glycerol for long-term storage. Protein levels were measured using JESS-Western Blot (Protein Simple). Total protein concentration was determined using the Pierce BCA Protein Assay Kit (Thermo Scientific, Cat #23225). Samples were run on a 25 capillary cartridge (12-240 kDa separation) on the chemiluminescent channel. Total protein was used as a loading control. A PRKAG2 anti-rabbit primary antibody (Sigma, diluted 1:12.5) and an anti-rabbit secondary antibody (Abcam, diluted 1:20) were used to detect the target protein. The Compass for Simple Western software was used to analyze the changes in protein expression.

### Cardiac Electrophysiology (ECG) in NHP

Electrocardiography (ECG) were taken using an electrocardiograph (Cardio 10, Bionet Co., Ltd., Korea), by the CRO Key Prime Research (KPR), in South Korea. The evaluation consisted of a quantitative measurement of the HR, QT- and PR-interval from a QRS complex of each recording. QTc was derived at each time point using Bazett's formula.

### Results

PRKAG2 mRNA and Protein Expression

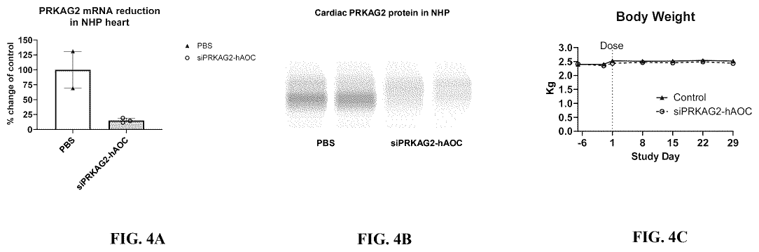

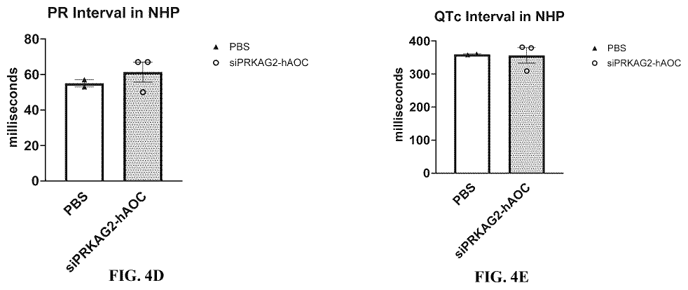

At 4 weeks following a single administration of 3 mg/kg siRNA dose of siPRKAG2-hAOC, PRKAG2 mRNA transcript expression levels were reduced in the heart, gastrocnemius, vastus lateralis, and biceps brachii, yet PRKAG2 mRNA levels were not reduced in the liver and kidney. Such results indicate that the pharmacologic activity of siPRKAG2-hAOC is selective to muscle tissue given the lack of PRKAG2 mRNA reduction in liver and kidney tissues at necropsy. In the gastrocnemius, vastus lateralis, and biceps brachii samples, approximately 50% reduction of PRKAG2 mRNA expression level was observed as early as 14 days post dosing and such reduction was maintained up to 28 days post dosing. In the heart samples, over 75% reduction of PRKAG2 mRNA expression level was observed on 29 days post dosing (FIG. 4A). Additionally, PRKAG2 protein expression level was reduced in heart after administration of a single 3 mg/kg dose of siPRKAG2-hAOC (FIG. 4B).

### Animal Body Weight and Cardiac Function

siPRKAG2-hAOC administration did not induce or result in any significant change in animal body weight over the entire duration of the study (FIG. 4C). Additionally, no significant differences on heart ECG parameters -the PR intervals and on the QTc intervals -were detected in the animal hearts (FIGS. 4D-4E), indicating the acceptable safety of siPRKAG2-hAOC.

### Example 8: In Vivo Evaluation of Intravenous and Subcutaneous Route of Administration of PRKAG2 hAOC in Non-Human Primates (NHP)

The nucleic acid sequences (5′ to 3′) of the guide/antisense strand and passenger strand/sense strand of human PRKAG2 (siPRKAG2) used in this example are present in Table 14. Base, sugar and phosphate modifications as shown in Table 14 were used to optimize the potency of the duplex and reduce immunogenicity. All siRNA strands were fully assembled on solid phase using standard phosphoramidite chemistry and purified over HPLC. Purified single strands were duplexed to get the double stranded siRNA. The passenger strand contained a conjugation handle, a C6—NH₂, at the 5′ end, which was connected to the siRNA passenger strand via phosphorothioate-inverted abasic-phosphorothioate linker. The human transferrin 1 receptor (hTfR1) mAb was conjugated to the siPRKAG2 to form siPRKAG2-hAOC DAR1 conjugates.

TABLE 14

| siRNA | siRNA Guide Sequence | siRNA Passenger Sequence |
|---|---|---|
| siPRKAG2 | VpUqsGfsAoAoUoAfAoAoUoAoUoCoUo | AosAosAoGoUoUoGoUoAfGfAfUoAoUoU |
| | AfCoAfAoCoUoUoUosUosUo (SEQ ID NO: | oUoAoUoUosCosAo (SEQ ID NO: 224) |
| | 212) | |
| Modification | 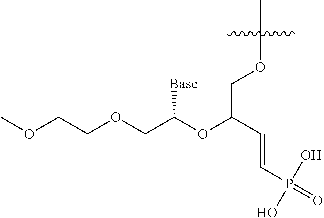 | 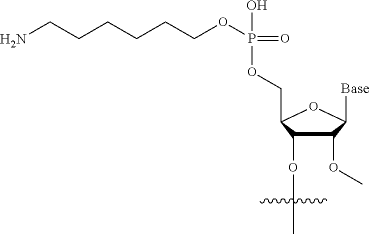 |
| | VpUq (base is uracil) | 5′ end of the passenger strand is |
| | | modified with 5am6_p_m. Sam6_p_m |
| | | structure connected to the first |
| | | nucleotide is shown above. Base is |
| | | Adenine in SEQ ID NO: 224 |

Xf refers to 2′-fluoro modified nucleotide where X is any base; Xo refers to 2′-O-methyl modified nucleotide, where X is any base; s refers to phosphorothioate internucleotide linkage.

### In Vivo Study Design

In this study, the pharmacodynamic (PD) profiles of siPRKAG2-hAOC, following a single subcutaneous (SC) or intravenous (IV) injection to cynomolgus monkeys, were evaluated. siPRKAG2-hAOC was composed of a human transferrin receptor 1 (TfR1) antibody conjugated to a small interfering ribonucleic acid (siRNA) targeting the human protein kinase AMP-activated non-catalytic subunit gamma 2 (PRKAG2) gene. PD profile was characterized by RT-qPCR analysis of PRKAG2 mRNA levels in heart, skeletal muscle, and non-skeletal muscles tissues.

### Methods:

Three female animals received a single dose of vehicle control or siPRKAG2-hAOC at 1 mg/kg (siRNA dose) via IV infusion over 60-minutes. The 1 mg/kg dose was also administered by SC injection (Table 15).

TABLE 15

| | | Dose Level | Dose Volume | Admin. | No. of | Terminal |
|---|---|---|---|---|---|---|
| Group | Test Article | (mg/kg) | (mL/kg) | Route | Females | Sacrifice |
| 1 | Sterile Saline | 0 | 10 | IV | 3 | Day 84 |
| 3 | siPRKAG2- | 1 | 10 | IV | 3 | Day 28 |
| | hAOC | | | | | |
| 4 | siPRKAG2- | 1 | 2 | SC | 3 | Day 28 |
| | hAOC | | | | | |

Punch biopsies of the gastrocnemius tissues were collected during acclimation (predose, corresponding to study Day -6), on Days 7, 21, and 28. Punch biopsies of the vastus lateralis were collected on Days 7, 21, and 28. Vastus lateralis muscle biopsies were collected on Days 7, 21, and 28. Heart (apex, LV free wall, and RV), biceps brachii, latissimus dorsi, liver, and kidney-cortex were collected at necropsy, on Day 28. Tissue samples were evaluated for PRKAG2 mRNA expression using quantitative polymerase chain reaction (RT-qPCR).

Tissue samples were evaluated for mRNA expression using a comparative RT-qPCR assay. Tissues samples ranging from 25-50 mg were homogenized in 1 mL of TRIzol (Thermo Fisher) on the OMNI Bead Ruptor Elite system (OMNI International). Total RNA was isolated from tissue homogenate supernatant using the Direct-zol-96 RNA kit 200 (Zymo Research) according to the manufacturer's instructions. 250 ng of purified RNA was reverse transcribed to cDNA using the HighCapacity cDNA Reverse Transcription Kit (Applied Biosystem) and SimpliAmp Thermal Cycler (Applied Biosystem). The amount of cDNA used per qPCR reaction was 50 ng (for muscle analysis) or 15 ng (for non-muscle tissue analysis). qPCR was performed using TaqMan Fast Universal Master Mix II (Thermo Fisher) and commercially available Tagman Assays. Each sample was run in technical duplicates in a Quant Studio instrument (Thermo Fisher). qPCR data was analyzed using the ΔΔCt method with gene of interest normalized to Peptidylprolyl isomerase B (PPIB) expression. PRKAG2 expression in gastroenemius and vastus lateralis was calculated using formula: 100*[2\hat{ }(ΔCt sample -ΔCt Predose), and in heart tissues, liver, kidney and biceps and latissimus dorsi was calculated using the formula 100*[2\hat{ }(ΔCt sample -ΔCt Sterile Saline Control), as stated, where Ct is the qPCR cycle threshold. Data was analyzed using GraphPad Prism 10 and is expressed as mean±SD.

A specific stem-loop real-time quantitative polymerase chain reaction (SL-RT-qPCR) assay was designed to measure the guide strand of siPRKAG2 in plasma and tissue homogenate. The SL-RT-qPCR assay along with the general primer and TaqMan probe was used. Briefly, calibration standards for an 8-point standard curve were generated by a 1:10 serial dilution of siPRKAG2 in assay diluent (TE buffer with 0.1% Triton-X) or TRIzol (Thermo Fisher). Study samples were diluted in assay diluent at the recommended Minimum Required Dilution (MRD; 1:100) or greater dilution levels. The calibration standards were diluted in assay diluent at the MRD. A siRNA-specific reverse transcription (RT) primer (custom from Integrated DNA Technologies) was added to the calibration standards and study samples, followed by an annealing and a reverse transcription step (Fisher Scientific, #43-665-97) on a thermocycler. The RT-qPCR products were quantified via conventional TaqMan RT-PCR (Fisher Scientific, #44-445-58) which included a siRNA-specific forward primer (custom from Integrated DNA Technologies), a universal reverse primer (custom from Integrated DNA Technologies), and a dye labeled siRNA-specific TaqMan probe (custom from Thermo Fisher). Raw Ct values were exported using the QuantStudio Real-Time PCR and Excel software. The standard curve of Ct values vs. log base 10 of corresponding siRNA concentrations was generated in GraphPad Prism 9.3.1. A simple linear regression of the siRNA standard curve was performed in GraphPad Prism, and the linear equation was used to interpolate study sample plasma and tissue concentrations in Excel. The lower limit of quantification was 5 μM. Sample values are reported as the average of technical replicates.

### Results

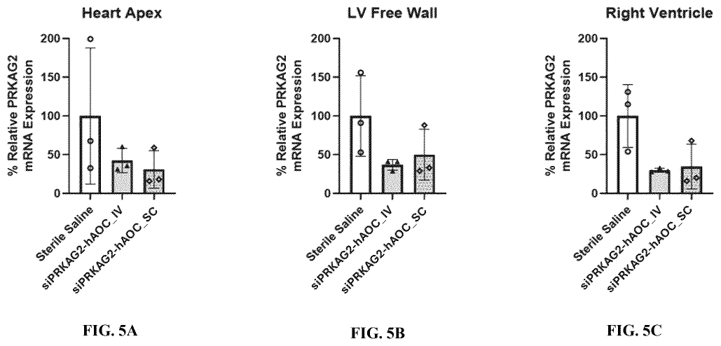

As shown in Table 16 and FIGS. 5A-C, following a single 1 mg/kg siRNA dose administration of siPRKAG2-hAOC by IV infusion, PRKAG2 mRNA transcript was reduced in the heart (apex, LV Free wall and RV) at day 28 to 42.6%, 37.2%, and 30.0%, respectively.-Following a single 1 mg/kg siRNA dose administration of siPRKAG2-hAOC by SC injection, PRKAG2 mRNA transcript was reduced in the heart (apex, LV Free wall and RV) at day 28 to 31.0%, 50.1%, and 34.6%, respectively.

TABLE 16

| | | | Dose | | | Heart Apex | | LV Free Wall | | RV | |
|---|---|---|---|---|---|---|---|---|---|---|---|
| | Test | Admin | Level | No. of | Study | Mean | | Mean | | Mean | |
| Group | Article | Route | (mg/kg) | Female | Day | %* | SD | %* | SD | %* | SD |
| 1 | Sterile | IV | 0 | 3 | 84 | 100.0 | 87.9 | 100.0 | 0.52 | 100.0 | 0.41 |
| | Saline | | | | | | | | | | |
| 3 | siPRKA | IV | 1 | 3 | 28 | 42.6 | 15.8 | 37.2 | 0.07 | 30.0 | 0.03 |
| | G2- | | | | | | | | | | |
| | hAOC | | | | | | | | | | |
| 4 | siPRKA | SC | 1 | 3 | 28 | 31.0 | 24.3 | 50.1 | 0.32 | 34.6 | 0.29 |
| | G2- | | | | | | | | | | |
| | hAOC | | | | | | | | | | |

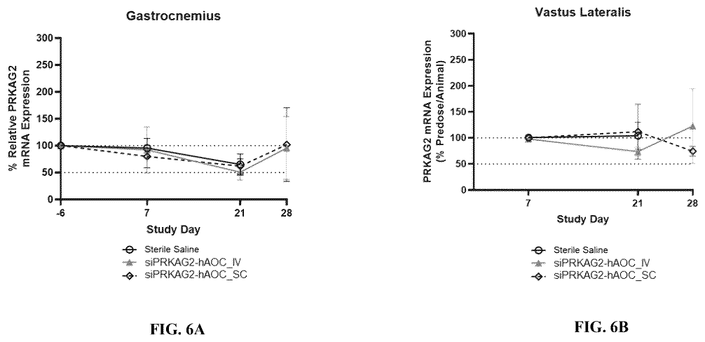

As shown in Table 17 and FIG. 6A, in gastronemius, PRKAG2 mRNA expression was reduced to 50% at Day 21 after single 1 mg/kg dose administration of siPRKAG2-hAOC, delivered IV or SC. As shown in Table 17 and FIG. 61B, in Vastus Lateralis, PRKAG2 mRNA expression was reduced to about 40% at Day 21, after single 1 mg/kg dose administration of siPRKAG2-hAOC, administered by IV.

TABLE 17

| | | | Dose | | | Gastrocnemius | | Vastus Lateralis | |
|---|---|---|---|---|---|---|---|---|---|
| | Test | Admin | Level | No. of | Study | Mean | | Mean | |
| Group | Article | Route | (mg/kg) | Female | Day | %* | SD | %* | SD |
| 1 | Sterile | IV | 0 | 3 | −6 | 100.0 | 0.0 | N.A. | N.A. |
| | Saline | | | 3 | 7 | 95.5 | 18.1 | 100.0 | 0.0 |
| | | | | 3 | 21 | 65.1 | 19.4 | 103.7 | 27.5 |
| 3 | siPRKAG2- | IV | 1 | 6 | −6 | 100.0 | 0.0 | N.A. | N.A. |
| | hAOC | | | 6 | 7 | 92.1 | 43.2 | 100.0 | 0.0 |
| | | | | 6 | 21 | 50.6 | 14.9 | 75.3 | 8.3 |
| | | | | 3 | 28 | 95.7 | 58.7 | 124.4 | 71.7 |
| 4 | siPRKAG2- | SC | 1 | 3 | −6 | 100.0 | 0.0 | N.A. | N.A. |
| | hAOC | | | 3 | 7 | 80.1 | 21.0 | 100.0 | 0.0 |
| | | | | 3 | 21 | 62.0 | 13.8 | 111.0 | 49.2 |
| | | | | 3 | 28 | 101.9 | 68.6 | 74.3 | 11.6 |

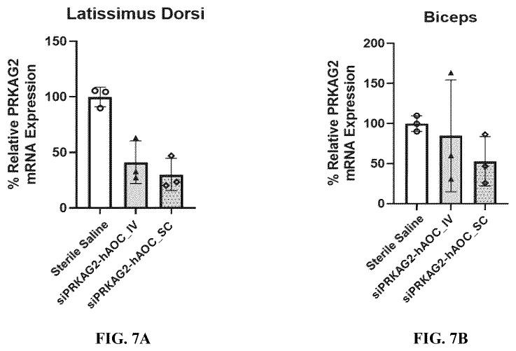

As shown in Table 18 and FIG. 7A, in Latissimus Dorsi, PRKAG2 mRNA expression was reduced to about 40% (IV injection) or to about 30% (SC injection) after 1 mg/kg dose of siPRKAG2-hAOC administration. In biceps, as shown in Table 18 and FIG. 71B, PRKAG2 mRNA expression was reduced to about 85% (IV injection) or to about 53% (SC injection) after 1 mg/kg dose of siPRKAG2-hAOC administration.

TABLE 18

| | | | Dose | | | | | | |
|---|---|---|---|---|---|---|---|---|---|
| | | Admin | Level | No. of | Study | Latissimus Dorsi | | Biceps | |
| Group | Test Article | Route | (mg/kg) | Female | Day | Mean | SD | Mean | SD |
| 1 | Sterile | IV | 0 | 3 | 84 | 100.00 | 8.77 | 100.00 | 9.77 |
| | Saline | | | | | | | | |
| 3 | siPRKAG2- | IV | 1 | 3 | 28 | 41.20 | 19.31 | 84.73 | 69.82 |
| | hAOC | | | | | | | | |
| 4 | siPRKAG2- | SC | 1 | 3 | 28 | 30.25 | 14.60 | 52.96 | 30.85 |
| | hAOC | | | | | | | | |

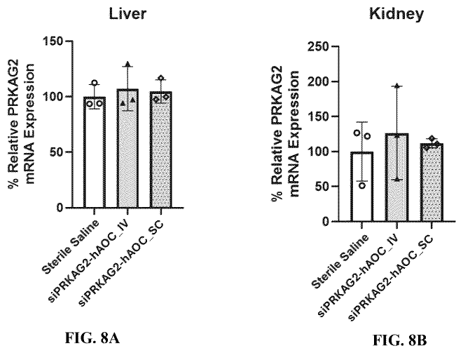

The pharmacologic activity of siPRKAG2-hAOC appeared to be selective to muscle tissue given the lack of PRKAG2 mRNA reduction in liver and kidney tissues (Table 19 and FIGS. 8A-B). Similar data were obtained in heart using a custom made PRKAG2 probe (PRKAG2_03) (data not shown) and using an additional cDNA synthesis kit (data not shown).

TABLE 19

| | Test | Admin | Dose Level | No. of | Study | Liver | | Kidney | |
|---|---|---|---|---|---|---|---|---|---|
| Group | Article | Route | (mg/kg) | Female | Day | Mean | SD | Mean | SD |
| 1 | Sterile | IV | 0 | 3 | 84 | 100.00 | 10.90 | 100.00 | 42.28 |
| | Saline | | | | | | | | |
| 3 | siPRKAG2- | IV | 1 | 3 | 28 | 107.23 | 19.88 | 126.51 | 66.83 |
| | hAOC | | | | | | | | |
| 4 | siPRKAG2- | SC | 1 | 3 | 28 | 104.73 | 10.48 | 111.74 | 6.49 |
| | hAOC | | | | | | | | |

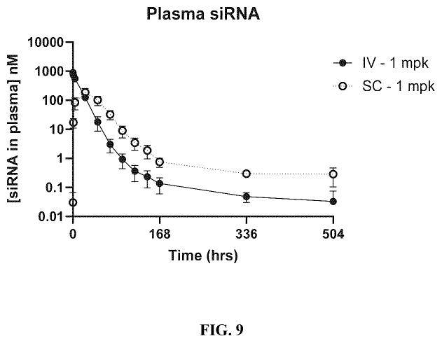

PRKAG2 concentrations in plasma following a single dose of siPRKiAG2-hAOC were also measured in the female animals in 168 hrs, 336 hrs, and 504 hrs after 1 mg/kg dose injection at Day 0. As shown in FIG. 9 and Table 20, rapid subcutaneous absorption of siPRKAG2-hAOC could be observed. The bioavailability of siPRKAG2-hAOC was about 80% measured by Tmax at approximately 24 hrs post dose.

TABLE 20

| | Dose | | Cmax | AUC last | CL |
|---|---|---|---|---|---|
| ROA | (mg/kg) | | (ng/mL) | (hr*ng/mL) | (mL/hr) |
| IV | 1 | Mean | 13,337 | 171,575 | 15.9 |
| | | SD | 1,514 | 13,160 | 1.1 |
| SC | 1 | Mean | 2,792 | 130,011 | 21.7* |
| | | SD | 948 | 36,573 | 5.7 |

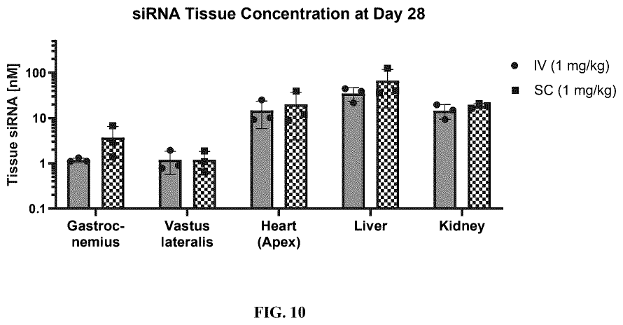

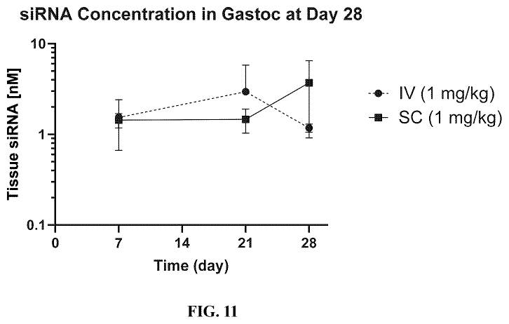

PRKAG2 siRNA concentrations in various tissues -gastrocnemius, vastus lateralis, heat apex, liver, and kidney -were measured at day 28 following a single dose of siPRKAG2-hAOC in the female animals. As shown in FIG. 10, siRNA concentrations remained in the tissues by subcutaneous injection were not substantially different from by intravenous injection indicating that similar target tissue delivery to muscle tissue was achieved in NHP with subcutaneous dosing of siPRKAG2-hAOC at 1 mg/kg compared to intravenous dosing.

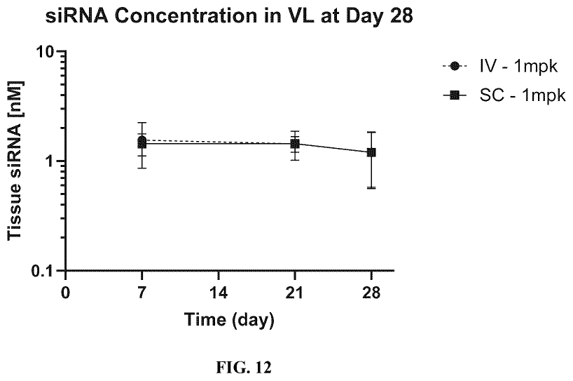

PRKAG2 siRNA concentrations could be also stably maintained in gastrocnemius tissues (FIG. 11) and vastus lateralis tissue (FIG. 12) at 7, 21, and 28 days post dose in both by intravenous injection and subcutaneous injection.

### Example 9: In Vivo Evaluation of Intravenous and Subcutaneous Route of Administration of siPRKAG2-mAOC in Mice

A 23-mer duplex siRNA with 21 bases of complementarity and 3′ dinucleotide overhangs was designed against human PRKAG2 (siPRKAG2) with the same nucleic acid sequences and modifications as described in Example 8. The mouse transferrin 1 receptor (mTfR1) mAb-siPRKAG2 DAR1 conjugates (siPRKAG2-mAOC) were generated, purified and characterized as described in Antibody siRNA Conjugate Synthesis Example 2. All conjugates were made through cysteine conjugation using a SMCC linker. Conjugates were characterized chromatographically.

In this example, the pharmacodynamic (PD) profiles of siPRKAG2-mAOC, following a single subcutaneous (SC) or intravenous (IV) injection in mice were evaluated. 4 groups of mice were administered with siPRKAG2-mAOC either subcutaneously (SC) or intravenously (IV) at a single dose of 2 mg/kg or 3 doses of 0.67 mg/kg (Table 21), and concentration of the siPRKAG2 in the plasma and various organs were evaluated. In addition, the efficacy/activity of the siPRKAG2-mAOC were evaluated by measuring the PRKAG2 expression levels in the muscle, heart and other tissues.

TABLE 21

| | | | | siRNA | | | | Terminal | |
|---|---|---|---|---|---|---|---|---|---|
| | | | siRNA: | Dose | Doses | | Survival | Bleed | |
| Group | Test Article | N | (mol/mol) | (mg/kg) | (#) | ROA | Bleeds | (Cardiac) | Harvest |
| 1 | mTfR1-mAb-siPRKAG2 | 4 | 1 | 2 | 1 | SC | 5 min, 1 h, | 24 h | 24 h |
| | DAR1 | | | | | | 4 h | | |
| 2 | mTfR1-mAb-siPRKAG2 | 4 | 1 | 2 | 1 | SC | 30 min, | 24 h | 15 d |
| | DAR1 | | | | | | 8 h, 72 h | | |
| 3 | mTfR1-mAb-siPRKAG2 | 4 | 1 | 2 | 1 | SC | 5 min, 1 h, | 24 h | 24 h |
| | DAR1 | | | | | | 4 h | | |
| 4 | mTfR1-mAb-siPRKAG2 | 4 | 1 | 2 | 1 | IV | 30 min, | 24 h | 15 d |
| | DAR1 | | | | | | 8 h, 72 h | | |
| 5 | mTfR1-mAb-siPRKAG2 | 4 | 1 | 0.67 | 3 | IV | 24 h, 32 h, | 15 d | 15 d |
| | DAR1 | | | | | | 48 h | | |
| 7 | PBS | 4 | | | 1 | SC | | | 15 d |

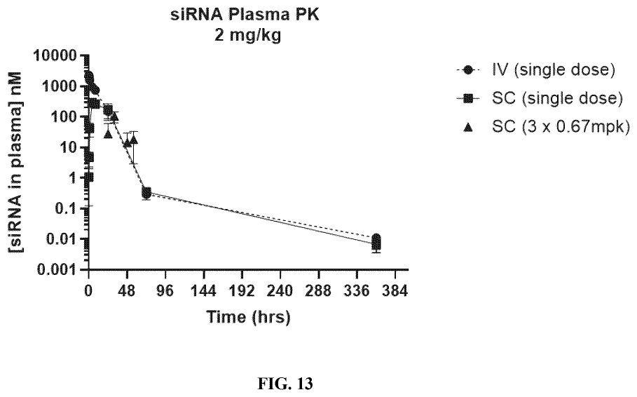

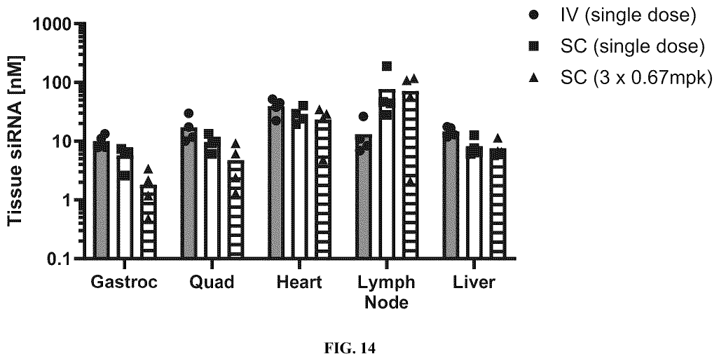

PRKAG2 concentrations in plasma following a 2 mg/kg single dose of siPRKAG2-mAOC or 0.67 mg/kg x 3 dose of siPRKAG2-mAOC were measured up to 15 days (360 hrs) after injection by IV or SC route at Day 0. As shown in FIG. 13, rapid subcutaneous absorption of siPRKAG2-mAOC was observed. The bioavailability of siPRKAG2-hAOC was about 46% measured by Tmax at approximately 4-8 hrs post dose.

PRKAG2 siRNA concentrations in various tissues -gastrocnemius, quad, heat apex, lymph node and liver-were measured at day 15 following a 2 mg/kg single dose administration of siPRKAG2-mAOC or 0.67 mg/kg x 3 dose administrations of siPRKAG2-mAOC. As shown in FIG. 14, siRNA concentrations remained in the tissues by subcutaneous injections were comparable with those by intravenous injection indicating that similar target tissue delivery to muscle tissue was achieved in mice with subcutaneous dosing of siPRKAG2-mAOC at 2 mg/kg single dose administration or 0.67 mg/kg x 3 dose administrations of siPRKAG2-mAOC.

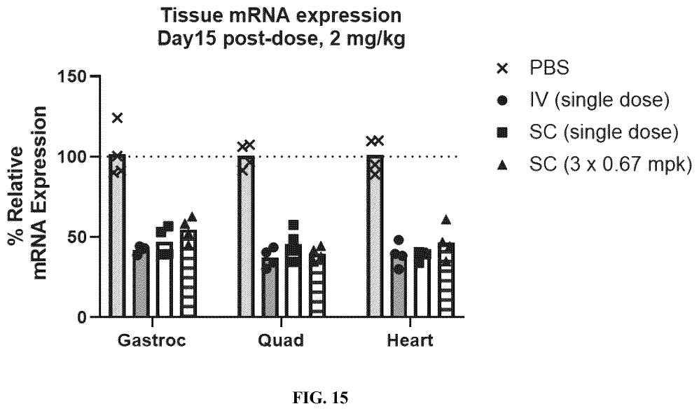

As shown in FIG. 15, following a single 2 mg/kg siRNA dose administration of siPRKAG2-mAOC by IV or SC, or 3x 0.67 mg/kg administrations by SC injections, PRKAG2 mRNA transcript expression level was reduced in gastroc and quad muscle tissues and heart by 50% or more at day 15. These results indicate that both IV and SC route of administrations of same dose of siPRKAG2-mAOC could exert substantially similar inhibitory effects to PRKAG2 mRNA expression in muscle tissues (skeletal and heart). Also these results further indicate that single injection and multiple SC injections administering the same final doses by SC route could achieve substantially same inhibitory effects to PRKAG2 expression in muscle tissues (skeletal and heart).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating cardiomyopathy in a subject in need thereof comprising:
administering the subject an effective amount of a polynucleotide conjugate, wherein the polynucleotide conjugate comprises an anti-transferrin receptor antibody or antigen-binding fragment thereof conjugated to a polynucleotide molecule, wherein the polynucleotide molecule hybridizes to a target sequence of PRKAG2 mRNA and mediates RNA interference against the PRKAG2 mRNA in a cardiac muscle cell, thereby treating cardiomyopathy in the subject.

2. The method of claim 1, wherein the cardiomyopathy is caused by a glycogen storage disease.

3. The method of claim 2, wherein the cardiomyopathy is caused by PRKAG2 syndrome or PRKAG2 cardiac syndrome.

4. The method of claim 3, wherein PRKAG2 syndrome or PRKAG2 cardiac syndrome is caused by a mutated PRKAG2 that has a gain of function.

5. The method of claim 4, wherein the mutated PRKAG2 comprises R531G mutation.

6. The method of claim 1, wherein the polynucleotide conjugate is administered via subcutaneous injections or intravenous injection.

7. The method of claim 1, wherein the polynucleotide molecule is a double-stranded inhibitory RNA molecule comprising a guide strand and a passenger strand, wherein the guide strand comprises a nucleic acid sequence having at least 17 consecutive nucleotides from a nucleic acid sequence selected from SEQ ID NOs: 58, 61, 62, 64, 77, and 98.

8. The method of claim 7, wherein the guide strand comprises a nucleic acid sequence having at least 17 consecutive nucleotides from a nucleic acid sequence selected from SEQ ID NOs: 209-220.

9. The method of claim 7, wherein the passenger strand comprises a nucleic acid sequence having at least 17 consecutive nucleotides from a nucleic acid sequence selected from SEQ ID NOs: 221-232.

10. The method of claim 1, wherein the polynucleotide conjugate reduces the mRNA expression level of the PRKAG2 mRNA in the cardiac muscle cell at least 40%, 50%, or 60% compared to PRKAG2 mRNA level before administration.

11. The method of claim 1, wherein the effective amount of the polynucleotide conjugate is effective to reduce the glycogen accumulation in the cardiac muscle cell of the subject.

12. The method of claim 1, wherein the polynucleotide conjugate is administered with a dose of about 1, 2, 3, or 4 mg/kg.

13. The method of claim 12, wherein the polynucleotide conjugate is administered with a dose of about 1-3 mg siRNA/kg intravenously.

14. The method of claim 12, wherein the polynucleotide conjugate is administered with a dose of about 1-3 mg siRNA/kg subcutaneously.

15. The method of claim 12, wherein the polynucleotide conjugate is administered with a dose of about 1 mg siRNA/kg intravenously.

16. The method of claim 12, wherein the polynucleotide conjugate is administered with a dose of about 1 mg siRNA/kg subcutaneously.

17. The method of claim 1, wherein the polynucleotide conjugate is administered once in about 1, 3, 5, 10, 12, 24, or 36 weeks.

18. The method of claim 17, wherein the polynucleotide conjugate is administered once in about 4, 8, 12, 16, 20, or 24 weeks.
